(19) United States
(12) Patent Application Publication
(10) Pub. No.: US 2010/0210524 A1
(43) Pub. Date: Aug. 19, 2010

(54) NOVEL ESTROGEN RECEPTOR LIGANDS

(76) Inventors: Theresa Apelqvist, Grodinge (SE); Joakim Löfstedt, Uppsala (SE); Thomas Norin, Sodertalje (SE); Mattias Wennerstål, Hagersten (SE); Xiongyu Wu, Huddinge (SE); Lars Hagberg, Stockholm (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 12/452,678

(22) PCT Filed: Jul. 21, 2008
(86) PCT No.: PCT/EP2008/005948

§ 371 Date: Jan. 14, 2010

(30) Foreign Application Priority Data
Jul. 20, 2007 (GB) 0714245.8
Feb. 29, 2008 (GB) 0803835.8

(51) Int. Cl. A61K 31/122; C07C 49/747; C07C 255/47; A61K 31/277; C07C 251/44; A61K 31/15; C07C 233/33; A61K 31/167; C07D 333/38; A61K 31/381; C07D 307/46; A61K 31/341; C07D 277/34; A61K 31/426; C07D 207/333; A61K 31/40; C07C 69/78; A61K 31/235; C07D 261/08; A61K 31/42; C07C 211/45; A61K 31/136; C07D 231/56; A61K 31/416; C07D 407/10; C07D 209/52; A61K 31/403; C07C 311/08; A61K 31/18; C07C 311/21; A61K 31/663; A61K 31/565; A61K 38/23; A61K 31/59; A61P 35/00; A61P 37/06; A61P 25/00; A61P 19/00; A61P 19/10; A61P 3/04; A61P 3/06; A61P 9/00; A61P 15/00

(52) U.S. Cl. 514/12; 568/327; 514/681; 558/426; 514/520; 564/265; 514/640; 564/222; 514/629; 549/61; 514/438; 549/498; 514/461; 548/187; 514/369; 548/560; 514/427; 560/108; 514/544; 548/247; 514/378; 564/428; 514/657; 548/361.1; 514/406; 548/515; 514/412; 564/99; 514/605; 564/92; 514/604; 514/102; 514/171; 514/167

(57) ABSTRACT

The invention provides a compound of formula (I) or a pharmaceutically acceptable ester, amide, solvate or salt thereof, including a salt of such an ester or amide, and a solvate of such an ester, amide or salt, wherein W, X, Y, Z, R¹, R², R⁷, R⁸, R⁹, R¹⁰, R¹¹, R¹², R¹³, R¹⁴, R¹⁵ and R¹⁶ are as defined in the specification. The invention also provides also provides the use of such compounds in the treatment or prophylaxis of a condition associated with a disease or disorder associated with estrogen receptor activity.

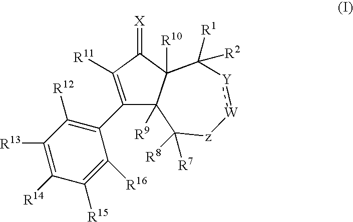

### FIELD OF INVENTION

This invention relates to compounds which are estrogen receptor ligands and are preferably selective for the estrogen receptor β isoform, to methods of preparing such compounds and to methods for using such compounds in treatment of diseases related to the estrogen receptor such as depressive disorders, anxiety disorders, Alzheimer's disease, cognitive disorders, osteoporosis, elevated blood triglyceride levels, atherosclerosis, endometriosis, urinary incontinence, autoimmune disease, and cancer of the lung, colon, breast, uterus and prostate.

### BACKGROUND OF INVENTION

The estrogen receptor (ER) is a ligand activated mammalian transcription factor involved in the up and down regulation of gene expression. The natural hormone for the estrogen receptor is β-17-estradiol (E2) and closely related metabolites. Binding of estradiol to the estrogen receptor causes a dimerization of the receptor and the dimer in turn binds to estrogen response elements (ERE's) on DNA. The ER/DNA complex recruits other transcription factors responsible for the transcription of DNA downstream from the ERE into mRNA which is eventually is translated into protein. Alternatively the interaction of ER with DNA may be indirect through the intermediacy of other transcription factors, most notably fos and jun. Since the expression of a large number of genes is regulated by the estrogen receptor and since the estrogen receptor is expressed in many cell types, modulation of the estrogen receptor through binding of either natural hormones or synthetic ER ligands can have profound effects on the physiology and pathophysiology of the organism.

Historically it has been believed there was only one estrogen receptor. However a second subtype (ER-β) has been discovered. While both the “classical” ER-α and the more recently discovered ER-β are widely distributed in different tissues, they nevertheless display markedly different cell type and tissue distributions. Therefore synthetic ligands which are either ER-α or ER-β selective may preserve the beneficial effects of estrogen while reducing the risk of undesirable side effects.

Estrogens are critical for sexual development in females. In addition, estrogens play an important role in maintaining bone density, regulation of blood lipid levels, and appear to have neuroprotective effects. Consequently decreased estrogen production in post-menopausal women is associated with a number of diseases such as osteoporosis, atherosclerosis, depression and cognitive disorders. Conversely certain types of proliferative diseases such as breast and uterine cancer and endometriosis are stimulated by estrogens and therefore antiestrogens (i.e., estrogen antagonists) have utility in the prevention and treatment of these types of disorders.

The efficacy of the natural estrogen, 17β-estradiol, for the treatment of various forms of depressive illness has also been demonstrated and it has been suggested that the anti-depressant activity of estrogen may be mediated via regulation of tryptophan hydroxylase activity and subsequent serotonin synthesis (See, e.g., Lu N Z, Shlaes T A, Cundlah C, Dziennis S E, Lyle R E, Bethea C L, “Ovarian steroid action on tryptophan hydroxylase protein and serotonin compared to localization of ovarian steroid receptors in midbrain of guinea pigs.” Endocrine 11:257-267, 1999). The pleiotropic nature of natural estrogen precludes its widespread, more chronic use due to the increased risk of proliferative effects on breast, uterine and ovarian tissues. The identification of the estrogen receptor, ERβ, has provided a means by which to identify more selective estrogen agents which have the desired anti-depressant activity in the absence of the proliferative effects which are mediated by ERα. Thus, it has been shown that therapeutic agents having ERβ-selectivity are potentially particularly effective in the treatment of depression.

What is needed in the art are compounds that can produce the same positive responses as estrogen replacement therapy without the negative side effects. Also needed are estrogen-like compounds that exert selective effects on different tissues of the body.

The compounds of the instant invention are ligands for estrogen receptors and as such may be useful for treatment or prevention of a variety of conditions related to estrogen functioning including bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, and lung, colon, breast, uterus, and prostate cancer.

A description of the synthesis of two series of estrogen receptor ligands is described in N. J. Clegg, S. Paruthiyil, D. C. Leitman and T. S. Scanlan, J. Med. Chem., 2005, 48, 5989-6003. These compounds are based on a common indene scaffold in an attempt to develop compounds that can selectively modulate ER-mediated transcription. The binding affinity of each of the compounds to ERα and ERβ was tested and several compounds were found to differentiate between ERα and ERβ subtypes at an estrogen receptor element (ERE), displaying various levels of partial to full agonist activity at ERα, while antagonizing estradiol action at ERβ.

### SUMMARY OF THE INVENTION

This invention provides a compound of formula (I) or a pharmaceutically acceptable ester, amide, solvate or salt thereof, including a salt of such an ester or amide, and a solvate of such an ester, amide or salt:

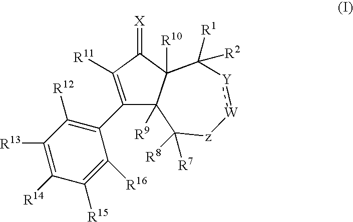

wherein
Y is selected from a bond, CR³R³⁰, C═CR³R³⁰ and NR³¹;
W is selected from a bond, CR⁴R⁴⁰, C═CR⁴R⁴⁰ and NR⁴¹;
and when both Y and W are not bond, then the bond between Y and W is a single bond or a double bond, and when it is a double bond Y is CR³ and W is CR⁴;
Z is selected from a bond, CR⁵R⁶ and C═CR⁵R⁶;
R¹, R², R³, R⁴, R⁵ R⁶, R⁷, R⁸, R³⁰ and R⁴⁰ are the same or are different and each is selected from the group consisting of hydrogen, OR^{D}, halogen, amino, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl, C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R³¹ and R⁴¹ are the same or are different and each is selected from the group consisting of hydrogen, OR^{A}, C₁₋₆ alkyl, C₂₋₆alkenyl, C₂₋₆ alkynyl, halo C₁₋₆ alkyl, dihalo C₁₋₆ alkyl, trihalo C₁₋₆ alkyl, C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆alkyl, dihalo C₁₋₆ alkyl and trihalo C₁₋₆ alkyl;
each R^{A} is independently selected from the group consisting of hydrogen, C₁₋₆ alkyl, C₂₋₆ alkenyl, C₂₋₆ alkynyl, C₃₋₈ cycloalkyl, C₃₋₈ cycloalkyl C₁₋₆ alkyl, phenyl, benzyl and C₅₋₈ heterocyclyl, each of said alkyl, alkenyl and alkynyl groups or parts of groups being optionally substituted with 1-3 substituents and each substituent is independently selected from the group consisting of OR^{A}, halogen, cyano and nitro; each of said cycloalkyl, phenyl, benzyl or C₅₋₈ heterocyclyl groups or parts of groups being optionally substituted with 1-3 substituents and each substituent is independently selected from the group consisting of OR^{A}, halogen, cyano, nitro, C₁₋₆ alkyl, C₂₋₆alkenyl, C₂₋₆ alkynyl, halo C₁₋₆ alkyl, dihalo C₁₋₆alkyl and trihalo C₁₋₆ alkyl;
each R^{D} is independently selected from the group consisting of C₁₋₆ alkyl, C₂₋₆ alkenyl, C₂₋₆ alkynyl, C₃₋₈ cycloalkyl, C₃₋₈ cycloalkyl C₁₋₆ alkyl, phenyl, benzyl and C₅₋₈ heterocyclyl, each of said alkyl, alkenyl and alkynyl groups or parts of groups being optionally substituted with 1-3 substituents and each substituent is independently selected from the group consisting of OR^{A}, halogen, cyano and nitro; each of said cycloalkyl, phenyl, benzyl or C₅₋₈ heterocyclyl groups or parts of groups being optionally substituted with 1-3 substituents and each substituent is independently selected from the group consisting of OR^{A}, halogen, cyano, nitro, C₁₋₆alkyl, C₂₋₆ alkenyl, C₂₋₆ alkynyl, halo C₁₋₆alkyl, dihalo C₁₋₆alkyl and tribal C₁₋₆ alkyl;
R⁹ and R¹⁰ are the same or different and each is selected from the group consisting of hydrogen, halogen, OR^{A}, C₁₋₆ alkyl, halo C₁₋₆ alkyl, dihalo C₁₋₆ alkyl and trihalo C₁₋₆ alkyl;
X is selected from O and NOR^{E};
R^{E} is selected from the group consisting of hydrogen, C₁₋₆ alkyl and phenyl;
R¹¹ is selected from the group consisting of hydrogen, halogen, cyano, OR^{A}, —C(O)C₁₋₄ alkyl, C₁₋₆ alkyl, halo C₁₋₆ alkyl, dihalo C₁₋₆ alkyl, trihalo C₁₋₆ alkyl, C₂₋₆ alkenyl, C₂₋₆ alkynyl, C₃₋₈ cycloalkyl, C₃₋₈ cycloalkyl C₁₋₆ alkyl, phenyl, benzyl and C₅₋₁₀heterocyclyl wherein said phenyl, benzyl or C₅₋₁₀ heterocyclyl group can either be unsubstituted or substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R¹² and R¹⁶ are the same or are different and each is selected from the group consisting of hydrogen, OR^{A}, halogen, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R¹³ and R¹⁵ are the same or different and each is selected from the group consisting of hydrogen, halogen, nitro, OR^{A}, N(R^{B})₂, C₁₋₆ alkyl, C₂₋₆ alkenyl, C₂₋₆ alkynyl, halo C₁₋₆ alkyl, dihalo C₁₋₆alkyl and trihalo C₁₋₆ alkyl;
R¹⁴ is selected from the group consisting of OR^{A}, N(R^{C})₂, —C(O)C₁₋₄ alkyl, —C(O)phenyl, and —O—C(O)R^{A}; or R¹⁴ and R¹⁵ or R¹³ and R¹⁴ may, together with the atoms they are attached to, form a 5-, 6- or 7-membered cyclic group optionally containing one to three heteroatoms selected from O, N and S, said 5-, 6- or 7-membered cyclic group being optionally substituted with one of more groups selected from OR^{A}, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
each R^{B} is independently selected from the group consisting of hydrogen, —C(O)C₁₋₄ alkyl, —C(O)phenyl, —SO₂C₁₋₄ alkyl, —SO₂phenyl, C₁₋₆ alkyl, C₂₋₆ alkenyl, C₂₋₆ alkynyl, C₃₋₈ cycloalkyl, C₃₋₈ cycloalkyl C₁₋₆ alkyl, phenyl, benzyl, C₅₋₁₀heterocyclyl and C₅₋₁₀heterocyclyl C₁₋₆ alkyl; and
each R^{C} is independently selected from the group consisting of hydrogen, —C(O)Me, C₁₋₆ alkyl, C₂₋₆ alkynyl, C₃₋₈ cycloalkyl, C₃₋₈ cycloalkyl C₁₋₆ alkyl, phenyl, benzyl, C₅₋₁₀heterocyclyl and C₅₋₁₀heterocyclyl C₁₋₆ alkyl.

Compounds of the invention have surprisingly been found to be ligands of the estrogen receptor. The compounds accordingly have use in the treatment or prophylaxis of conditions associated with estrogen receptor activity.

### DETAILED DESCRIPTION OF INVENTION

The compounds of formula (I) may contain stereogenic centres, stereogenic axes, and stereogenic planes (as described in: E. L. Eliel and S. H. Wilen, Stereochemistry of Carbon Compounds, John Wiley & Sons, New York, 1994, pages 1119-1190), and occur as racemic mixtures, scalemic mixtures, and as individual diastereomers, with all possible isomers, including optical isomers (enantiomers), and mixtures of these, being included within the scope of the present invention. In addition, the compounds disclosed herein may exist as tautomers and both tautomeric forms are intended to be encompassed by the scope of the invention, even though only one tautomeric structure is depicted.

The present invention provides compounds that are estrogen receptor ligands and have the general formula (I) as described above. The term “estrogen receptor ligand” as used herein is intended to cover any moiety which binds to an estrogen receptor. The ligand may act as an agonist, a partial agonist, an antagonist or a partial antagonist. Ligands are classified as “full agonists” if they display efficacy >60% in a dose-response assay, and as “partial agonists” if they display efficacy of 10-59%. Ligands that are able to abolish the agonist activity of estradiol in competition assays i.e. inhibit to basal activity levels are termed “full antagonists”. Ligands that inhibit agonist activity of estradiol in competition assays down to the level of partial activation are termed “partial antagonists”. The ligand may be ERβ selective or display mixed ERα and ERβ activity. For example, the ligand may act both as an agonist or a partial agonist of ERβ and as an antagonist or a partial antagonist of ERα. Preferred compounds of the invention are ERβ selective. Preferred compounds of the invention are full agonists or partial agonists, preferably full agonists.

In one embodiment, the invention provides a compound of formula (I) as described above wherein:

Y is selected from a bond or C R³R³⁰;
W is selected from a bond or C R⁴R⁴⁰;
and when both Y and W are not bond, then the bond between Y and W is a single bond or a double bond, and when it is a double bond Y is CR³ and W is CR⁴;
Z is selected from a bond or CR⁵R⁶;
R¹, R², R³, R⁴, R⁵, R⁶, R⁷, R⁸, R³⁰ and R⁴⁰ are the same or are different and each is selected from the group consisting of hydrogen, OR^{A}, halogen, amino, cyano, nitro, C₁₋₆ alkyl, C₂₋₆alkenyl, C₂₋₆ alkynyl, halo C₁₋₆ alkyl, dihalo C₁₋₆ alkyl and trihalo C₁₋₆alkyl, C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is independently selected from the group consisting of OR^{A}, halogen, cyano, nitro, C₁₋₆alkyl, C₂₋₆alkenyl, C₂₋₆ alkynyl, halo C₁₋₆alkyl, dihalo C₁₋₆ alkyl and trihalo C₁₋₆ alkyl;
each R^{A} is independently selected from the group consisting of hydrogen, C₁₋₆ alkyl, C₂₋₆ alkenyl, C₂₋₆ alkynyl, C₃₋₈ cycloalkyl, C₃₋₈ cycloalkyl C₁₋₆ alkyl, phenyl, benzyl and C₅₋₈heterocyclyl;
R⁹ and R¹⁰ are the same or different and each is selected from the group consisting of hydrogen, halogen, OR^{A}, C₁₋₆ alkyl, halo C₁₋₆alkyl, dihalo C₁₋₆ alkyl and trihalo C₁₋₆alkyl;
X is selected from O and NOH;
R¹¹ is selected from the group consisting of hydrogen, halogen, cyano, OR^{A}, —C(O)C₁₋₄ alkyl, C₁₋₆alkyl, halo C₁₋₆alkyl, dihalo C₁₋₆alkyl, trihalo C₁₋₆alkyl, C₂₋₆alkenyl, C₂₋₆ alkynyl, C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;

R¹² and R¹⁶ are the same or are different and each is selected from the group consisting of hydrogen, OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;

R¹³ and R¹⁵ are the same or different and each is selected from the group consisting of hydrogen, halogen, cyano, nitro, OR^{A}, N(R^{B})₂, C₁₋₆alkyl, C₂₋₆ alkenyl, C₂₋₆ alkynyl, halo C₁₋₆alkyl, dihalo C₁₋₆alkyl and trihalo C₁₋₆ alkyl;
R¹⁴ is selected from the group consisting of OR^{A}, N(R^{B})₂, —C(O)C₁₋₄ alkyl, —C(O)phenyl, and —O—C(O)R^{A}; or R¹⁴ and R¹⁵ or R¹³ and R¹⁴ may, together with the atoms they are attached to, form a 5-, 6- or 7-membered cyclic group optionally containing one to three heteroatoms selected from O, N and S; and
each R^{B} is independently selected from the group consisting of hydrogen, —C(O)C₁₋₄ alkyl, —C(O)phenyl, C₁₋₆alkyl, C₂₋₆alkenyl, C₂₋₆alkynyl, C₃₋₈ cycloalkyl, C₃₋₈ cycloalkyl C₁₋₆alkyl, phenyl, benzyl and C₅₋₈ heterocyclyl.

In one embodiment of the invention, Y is CR³R³⁰, W is CR⁴R⁴⁰, and the bond between Y and W is a single bond. In this embodiment, Z is a bond or CR⁵R⁶; preferably Z is a bond.

In another embodiment, Y is CR³, W is CR⁴, and the bond between Y and W is a double bond. In this embodiment, Z is a bond or CR⁵R⁶; preferably Z is a bond.

In a further embodiment, Y is CR³R³⁰, W is a bond, and Z is a bond. Accordingly, in a preferred embodiment, the invention provides a compound of formula (Ia) or a pharmaceutically acceptable ester, amide, solvate or salt thereof, including a salt of such an ester or amide, and a solvate of such an ester, amide or salt:

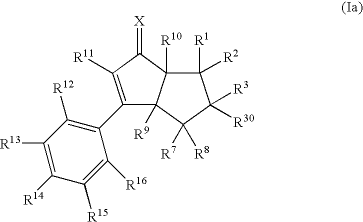

wherein X, R¹, R², R³, R⁷, R⁸, R⁹, R¹⁰, R¹¹, R¹², R¹³, R¹⁴, R¹⁵, R¹⁶ and R³⁰ are as defined for compounds of formula (I).

Preferably, R¹, R², R³, R⁴, R⁵, R⁶, R⁷, R⁸, R³⁰ and R⁴⁰ are the same or are different and each is selected from the group consisting of hydrogen, OR^{D}, halogen, amino, cyano, nitro, C₁₋₄ alkyl, halo C₁₋₄ alkyl, dihalo C₁₋₄ alkyl and trihalo C₁₋₄ alkyl. More preferably, R¹, R², R³, R⁴, R⁵, R⁶, R⁷, R⁸, R³⁰ and R⁴⁰ are the same or are different and each is selected from the group consisting of hydrogen, OR^{A}, halogen, C₁₋₄ alkyl, halo C₁₋₄ alkyl, dihalo C₁₋₄ alkyl and trihalo C₁₋₄ alkyl. Most preferably, R¹, R², R³, R⁴, R⁵, R⁶, R⁷, R⁸, R³⁰ and R⁴⁰ are the same or are different and each is selected from the group consisting of hydrogen, and C₁₋₂ alkyl. Preferably, R³ is hydrogen or C₁₋₂ alkyl.

Preferably, R³¹ and R⁴¹ are the same or are different and each is selected from the group consisting of hydrogen, C₁₋₆ alkyl, C₂₋₆ alkenyl, C₂₋₆ alkynyl, halo C₁₋₆ alkyl, dihalo C₁₋₆ alkyl, trihalo C₁₋₆ alkyl, C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl. More preferably, R³¹ and R⁴¹ are the same or are different and each is selected from the group consisting of hydrogen, C₁₋₆ alkyl, halo C₁₋₆ alkyl, dihalo C₁₋₆ alkyl and trihalo C₁₋₆ alkyl.

Preferably, each R^{A} is independently selected from the group consisting of hydrogen, C₁₋₄ alkyl, C₃₋₈ cycloalkyl, C₃₋₈ cycloalkyl C₁₋₂ alkyl, phenyl and benzyl. More preferably, each R^{A} is independently selected from the group consisting of hydrogen, C₁₋₄ alkyl, C₃₋₆ cycloalkyl, phenyl and benzyl. Most preferably, each R^{A} is independently selected from the group consisting of hydrogen and C₁₋₄ alkyl.

Preferably, each R^{D} is independently selected from the group consisting of C₁₋₄ alkyl, C₃₋₈ cycloalkyl, C₃₋₈ cycloalkyl C₁₋₂ alkyl, phenyl and benzyl. More preferably, each R^{D} is independently selected from the group consisting of C₁₋₄ alkyl, C₃₋₆ cycloalkyl, phenyl and benzyl. Most preferably, each R^{D} is C₁₋₄ alkyl.

Preferably, R⁹ and R¹⁰ are the same or different and each is selected from the group consisting of hydrogen, halogen, OR^{A}, C₁₋₄ alkyl, halo C₁₋₄ alkyl, dihalo C₁₋₄ alkyl and trihalo C₁₋₄ alkyl. More preferably, R⁹ is selected from the group consisting of C₁₋₄ alkyl, halo C₁₋₄ alkyl, dihalo C₁₋₄ alkyl and trihalo C₁₋₄ alkyl. Most preferably, R⁹ is C₁₋₄ alkyl. More preferably, R¹⁰ is selected from the group consisting of hydrogen and halogen. Most preferably, R¹⁰ is hydrogen or fluoro.

In one embodiment, X is O.

In another embodiment, X is NOR^{E}.

Preferably, R^{E} is selected from the group consisting of hydrogen, C₁₋₄ alkyl and phenyl.

Preferably, R¹¹ is selected from the group consisting of hydrogen, halogen, cyano, —C(O)C₁₋₄ alkyl, C₁₋₄ alkyl, halo C₁₋₄ alkyl, dihalo C₁₋₄ alkyl, trihalo C₁₋₄ alkyl, C₂₋₆ alkenyl, C₂₋₄ alkynyl, C₃₋₆ cycloalkyl, C₃₋₆ cycloalkyl C₁₋₂ alkyl, phenyl, benzyl and C₅₋₆ heterocyclyl wherein said phenyl, benzyl or C₅₋₆ heterocyclyl group can either be unsubstituted or substituted with 1-3 substituents and each substituent is independently selected from the group consisting of OR^{A}, halogen, cyano, nitro, C₁₋₂ alkyl, halo C₁₋₂ alkyl, dihalo C₁₋₂ alkyl and trihalo C₁₋₂ alkyl. More preferably, R¹¹ is selected from the group consisting of hydrogen, halogen, cyano, —C(O)C₁₋₂ alkyl, C₁₋₂ alkyl, halo C₁₋₂ alkyl, dihalo C₁₋₂ alkyl, trihalo C₁₋₂ alkyl, C₂₋₆ alkenyl, C₂₋₄ alkynyl, C₃₋₆ cycloalkyl, phenyl and C₅ heterocyclyl wherein said phenyl or C₅ heterocyclyl group can either be unsubstituted or substituted with 1-2 substituents and each substituent is independently selected from the group consisting of OR^{A}, halogen, cyano, nitro, methyl and trifluoromethyl. Most preferably, R¹¹ is selected from the group consisting of halogen, cyano, —C(O)C₁₋₂ alkyl, C₁₋₂ alkyl, halo C₁₋₂ alkyl, dihalo C₁₋₂ alkyl, trihalo C₁₋₂ alkyl, C₃₋₆ cycloalkyl, phenyl and C₅ heterocyclyl wherein said phenyl or C₅ heterocyclyl group can either be unsubstituted or substituted with 1-2 substituents and each substituent is independently selected from the group consisting of OR^{A}, halogen, cyano, nitro, methyl and trifluoromethyl. Preferred C₅ heterocyclyl groups include furanyl, thiophenyl, pyrrolyl, and thiazolyl.

Preferably, R¹² and R¹⁶ are the same or are different and each is selected from the group consisting of hydrogen, OR^{A}, halogen, nitro, C₁₋₄ alkyl, halo C₁₋₄ alkyl, dihalo C₁₋₄ alkyl and trihalo C₁₋₄ alkyl. More preferably, R¹² and R¹⁶ are the same or are different and each is selected from the group consisting of hydrogen, OR^{A}, halogen, C₁₋₄ alkyl, halo C₁₋₄ alkyl, dihalo C₁₋₄ alkyl and trihalo C₁₋₄ alkyl. Most preferably, R¹² and R¹⁶ are the same or are different and each is selected from the group consisting of hydrogen and halogen.

Preferably, R¹³ and R¹⁵ are the same or different and each is selected from the group consisting of hydrogen, halogen, nitro, OR^{A}, N(R^{B})₂, C₁₋₄ alkyl, halo C₁₋₄alkyl, dihalo C₁₋₄ alkyl and trihalo C₁₋₄ alkyl. More preferably, R¹³ and R¹⁵ are the same or different and each is selected from the group consisting of hydrogen, halogen, OR^{A}, C₁₋₄ alkyl, halo C₁₋₄alkyl, dihalo C₁₋₄ alkyl and trihalo C₁₋₄ alkyl. Most preferably, R¹³ and R¹⁵ are the same or different and each is selected from the group consisting of hydrogen and halogen.

Preferably, R¹⁴ is selected from the group consisting of OR^{A}, N(R^{C})₂, —C(O)C₁₋₄ alkyl, —C(O)phenyl, and —O—C(O)R^{A}. More preferably, R¹⁴ is selected from the group consisting of OR^{A}, N(R^{C})₂, —OC(O)C₁₋₄ alkyl, and —OC(O)phenyl. Most preferably, R¹⁴ is selected from the group consisting of OR^{A}, —OC(O)C₁₋₄ alkyl, and —OC(O)phenyl.

Preferably, each R^{B} is independently selected from the group consisting of hydrogen, —C(O)C₁₋₄ alkyl, C₁₋₄ alkyl, C₃₋₆ cycloalkyl, C₃₋₆ cycloalkyl C₁₋₂ alkyl, phenyl and benzyl. More preferably, each R^{B} is independently selected from the group consisting of hydrogen, —C(O)C₁₋₄ alkyl, and C₁₋₄ alkyl. Most preferably, each R^{B} is independently selected from the group consisting of hydrogen, —C(O)methyl, and C₁₋₂ alkyl;

Preferably, each R^{C} is independently selected from the group consisting of hydrogen, —C(O)Me, C₁₋₄ alkyl, C₃₋₆ cycloalkyl, C₃₋₆ cycloalkyl C₁₋₂ alkyl, phenyl and benzyl. More preferably, each R^{C} is independently selected from the group consisting of hydrogen, —C(O)Me, and C₁₋₄ alkyl. Most preferably, each R^{C} is independently selected from the group consisting of hydrogen, —C(O)Me, and C₁₋₂ alkyl.

Accordingly, the invention provides a compound of formula (I) wherein

Y is selected from a bond, CR³R³⁰ and C═CR³R³⁰;
W is selected from a bond, CR⁴R⁴⁰ and C═CR⁴R⁴⁰;
and when both Y and W are not bond, then the bond between Y and W is a single bond or a double bond, and when it is a double bond Y is CR³ and W is CR⁴;
Z is selected from a bond or CR⁵R⁶;
R¹, R², R³, R⁴, R⁵, R⁶, R⁷, R⁸, R³⁰ and R⁴⁰ are the same or are different and each is selected from the group consisting of hydrogen, OR^{D}, halogen, C₁₋₄ alkyl, halo C₁₋₄ alkyl, dihalo C₁₋₄ alkyl and trihalo C₁₋₄ alkyl;
each R^{A} is independently selected from the group consisting of hydrogen, C₁₋₄ alkyl, C₃₋₆ cycloalkyl, phenyl and benzyl;
each R^{D} is independently selected from the group consisting of C₁₋₄ alkyl, C₃₋₆ cycloalkyl, phenyl and benzyl;
R⁹ and R¹⁰ are the same or different and each is selected from the group consisting of hydrogen, halogen, OR^{A}, C₁₋₄ alkyl, halo C₁₋₄ alkyl, dihalo C₁₋₄ alkyl and trihalo C₁₋₄ alkyl;
X is selected from O and NOH;
R¹¹ is selected from the group consisting of hydrogen, halogen, cyano, —C(O)C₁₋₄ alkyl, C₁₋₄ alkyl, halo C₁₋₄ alkyl, dihalo C₁₋₄ alkyl, trihalo C₁₋₄ alkyl, C₂₋₆ alkenyl, C₂₋₄ alkynyl, C₃₋₆ cycloalkyl, C₃₋₆ cycloalkyl C₁₋₂ alkyl, phenyl, benzyl and C₅₋₆ heterocyclyl wherein said phenyl, benzyl or C₅₋₆ heterocyclyl group can either be unsubstituted or substituted with 1-3 substituents and each substituent is independently selected from the group consisting of OR^{A}, halogen, cyano, nitro, C₁₋₂ alkyl, halo C₁₋₂ alkyl, dihalo C₁₋₂ alkyl and trihalo C₁₋₂ alkyl;

R¹² and R¹⁶ are the same or are different and each is selected from the group consisting of hydrogen, OR^{A}, halogen, C₁₋₄ alkyl, halo C₁₋₄ alkyl, dihalo C₁₋₄ alkyl and trihalo C₁₋₄ alkyl;

R¹³ and R¹⁵ are the same or different and each is selected from the group consisting of hydrogen, halogen, OR^{A}, N(R^{B})₂, C₁₋₄ alkyl, halo C₁₋₄ alkyl, dihalo C₁₋₄ alkyl and trihalo C₁₋₄ alkyl;
R¹⁴ is selected from the group consisting of hydrogen, OR^{A}, N(R^{C})₂, —C(O)C₁₋₄ alkyl, —C(O)phenyl, and —O—C(O)R^{A} or R¹⁴ and R¹⁵ or R¹³ and R¹⁴ may, together with the atoms they are attached to, form a 5-, 6- or 7-membered cyclic group optionally containing one to three heteroatoms selected from O and N;
each R^{B} is independently selected from the group consisting of hydrogen, —C(O)C₁₋₄ alkyl, and C₁₋₄ alkyl; and
each R^{C} is independently selected from the group consisting of hydrogen, —C(O)Me, and C₁₋₄ alkyl.

The invention also provides a compound of formula (I) wherein

Y is selected from a bond and CR³R³⁰;
W is selected from a bond and CR⁴R⁴⁰;
and when both Y and W are not bond, then the bond between Y and W is a single bond or a double bond, and when it is a double bond Y is CR³ and W is CR⁴;
Z is selected from a bond or CR⁵R⁶;
R¹, R², R³, R⁴, R⁵, R⁶, le, R⁸, R³⁰ and R⁴⁰ are the same or are different and each is selected from the group consisting of hydrogen, OR^{D}, halogen, C₁₋₄ alkyl, halo C₁₋₄ alkyl, dihalo C₁₋₄ alkyl and trihalo C₁₋₄alkyl;
each R^{A} is independently selected from the group consisting of hydrogen, C₁₋₄ alkyl, C₃₋₆ cycloalkyl, phenyl and benzyl;
each R^{D} is independently selected from the group consisting of C₁₋₄ alkyl, C₃₋₆ cycloalkyl, phenyl and benzyl;
R⁹ and R¹⁰ are the same or different and each is selected from the group consisting of hydrogen, halogen, OR^{A}, C₁₋₄ alkyl, halo C₁₋₄ alkyl, dihalo C₁₋₄ alkyl and trihalo C₁₋₄ alkyl;
X is selected from O and NOH;

R¹¹ is selected from the group consisting of hydrogen, halogen, cyano, —C(O)C₁₋₄ alkyl, C₁₋₄ alkyl, halo C₁₋₄ alkyl, dihalo C₁₋₄ alkyl, trihalo C₁₋₄ alkyl, C₂₋₆ alkenyl, C₂₋₄ alkynyl, C₃₋₆ cycloalkyl, C₃₋₆ cycloalkyl C₁₋₂ alkyl, phenyl, benzyl and C₅₋₆heterocyclyl wherein said phenyl, benzyl or C₅₋₆heterocyclyl group can either be unsubstituted or substituted with 1-3 substituents and each substituent is independently selected from the group consisting of OR^{A}, halogen, cyano, nitro, C₁₋₂ alkyl, halo C₁₋₂ alkyl, dihalo C₁₋₂ alkyl and trihalo C₁₋₂ alkyl;

R¹² and R¹⁶ are the same or are different and each is selected from the group consisting of hydrogen, OR^{A}, halogen, C₁₋₄ alkyl, halo C₁₋₄ alkyl, dihalo C₁₋₄ alkyl and trihalo C₁₋₄ alkyl;
R¹³ and R¹⁵ are the same or different and each is selected from the group consisting of hydrogen, halogen, OR^{A}, N(R^{B})₂, C₁₋₄ alkyl, halo C₁₋₄alkyl, dihalo C₁₋₄ alkyl and trihalo C₁₋₄ alkyl;
R¹⁴ is selected from the group consisting of hydrogen, OR^{A}, N(R^{C})₂, —C(O)C₁₋₄ alkyl, —C(O)phenyl, and —O—C(O)R^{A} or R¹⁴ and R¹⁵ or R¹³ and R¹⁴ may, together with the atoms they are attached to, form a 5-, 6- or 7-membered cyclic group optionally containing one to three heteroatoms selected from O and N;
each R^{B} is independently selected from the group consisting of hydrogen, —C(O)C₁₋₄ alkyl, and C₁₋₄ alkyl; and
each R^{C} is independently selected from the group consisting of hydrogen, —C(O)Me, and C₁₋₄ alkyl.

The invention also provides a compound of formula (Ia) or a pharmaceutically acceptable ester, amide, solvate or salt thereof, including a salt of such an ester or amide, and a solvate of such an ester, amide or salt:

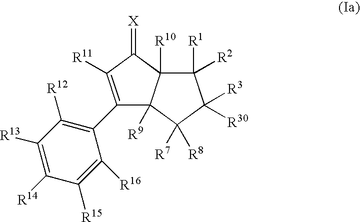

wherein
R¹, R², R³, R⁸ and R³⁰ are the same or are different and each is selected from the group consisting of hydrogen, halogen, C₁₋₄ alkyl, halo C₁₋₄ alkyl, dihalo C₁₋₄ alkyl and trihalo C₁₋₄ alkyl;
each R^{A} is independently selected from the group consisting of hydrogen, C₁₋₄ alkyl, C₃₋₆ cycloalkyl, phenyl and benzyl;
R⁹ and R¹⁰ are the same or different and each is selected from the group consisting of hydrogen, halogen, C₁₋₄alkyl, halo C₁₋₄ alkyl, dihalo C₁₋₄ alkyl and trihalo C₁₋₄ alkyl;
X is selected from O and NOH;
R¹¹ is selected from the group consisting of hydrogen, halogen, cyano, —C(O)C₁₋₄ alkyl, C₁₋₄ alkyl, halo C₁₋₄ alkyl, dihalo C₁₋₄ alkyl, trihalo C₁₋₄ alkyl, C₂₋₆ alkenyl, C₂₋₄ alkynyl, C₃₋₆ cycloalkyl, C₃₋₆ cycloalkyl C₁₋₂ alkyl, phenyl, benzyl and C₅₋₆heterocyclyl wherein said phenyl, benzyl or C₅₋₆ heterocyclyl group can either be unsubstituted or substituted with 1-3 substituents and each substituent is independently selected from the group consisting of OR^{A}, halogen, cyano, nitro, C₁₋₂ alkyl, halo C₁₋₂ alkyl, dihalo C₁₋₂ alkyl and trihalo C₁₋₂ alkyl;
R¹² and R¹⁶ are the same or are different and each is selected from the group consisting of hydrogen, halogen, C₁₋₄ alkyl, halo C₁₋₄ alkyl, dihalo C₁₋₄ alkyl and trihalo C₁₋₄ alkyl;
R¹³ and R¹⁵ are the same or different and each is selected from the group consisting of hydrogen, halogen, C₁₋₄ alkyl, halo C₁₋₄alkyl, dihalo C₁₋₄ alkyl and trihalo C₁₋₄alkyl;
R¹⁴ is selected from the group consisting of OR^{A}, N(R^{C})₂, and —O—C(O)R^{A}; and
each R^{C} is independently selected from the group consisting of hydrogen, —C(O)Me, and C₁₋₄ alkyl.

Preferred enantiomeric forms of the compounds of the invention have the following stereochemistry:

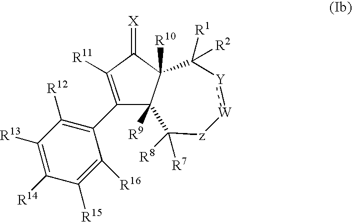

Compounds of the invention include, but are not limited to, the following:
3-(4-hydroxy-phenyl)-2-phenyl-4,5,6,6a-tetrahydro-3aH-pentalen-1-one (E1);
2-bromo-3-(4-hydroxy-phenyl)-4,5,6,6a-tetrahydro-3aH-pentalen-1-one (E2);
(3aR,6aS)-2-bromo-3-(4-hydroxy-phenyl)-4,5,6,6a-tetrahydro-3aH-pentalen-1-one (E3);
(3aS,6aR)-2-bromo-3-(4-hydroxy-phenyl)-4,5,6,6a-tetrahydro-3aH-pentalen-1-one (E4);
2-bromo-3-(4-hydroxy-phenyl)-5-methyl-4,5,6,6a-tetrahydro-3aH-pentalen-1-one (E5);
2-bromo-5-ethyl-3-(4-hydroxy-phenyl)-4,5,6,6a-tetrahydro-3aH-pentalen-1-one (E6);
2-chloro-3-(4-hydroxy-phenyl)-4,5,6,6a-tetrahydro-3aH-pentalen-1-one (E7);
3-(4-hydroxy-phenyl)-1-oxo-1,3a,4,5,6,6a-hexahydro-pentalene-2-carbonitrile (E8);
3-(4-hydroxy-phenyl)-2-trifluoromethyl-4,5,6,6a-tetrahydro-3aH-pentalen-1-one (E9);
2-cyclopropyl-3-(4-hydroxy-phenyl)-4,5,6,6a-tetrahydro-3aH-pentalen-1-one (E10);
2,2-dimethyl-propionic acid 4-(2-bromo-3-oxo-3,3a,4,5,6,6a-hexahydro-pentalen-1-yl)-phenyl ester (E11);
2-bromo-6a-fluoro-3-(4-hydroxy-phenyl)-4,5,6,6a-tetrahydro-3aH-pentalen-1-one (E12);
2-bromo-3-(4-hydroxy-phenyl)-6a-methyl-4,5,6,6a-tetrahydro-3aH-pentalen-1-one (E13);
3-(4-hydroxy-phenyl)-3a,4,7,7a-tetrahydro-inden-1-one (E14);
3-(4-hydroxy-phenyl)-3a,4,5,6,7,7a-hexahydro-inden-1-one (E15);
2-bromo-3-(4-hydroxy-phenyl)-3a,4,5,6,7,7a-hexahydro-inden-1-one (E16);
2-bromo-3-(4-hydroxy-phenyl)-4,5,6,6a-tetrahydro-3aH-pentalen-1-one oxime (E17);
N-[4-(2-bromo-3-oxo-3,3a,4,5,6,6a-hexahydro-pentalen-1-yl)-phenyl]-acetamide (E 18);
3-(4-hydroxy-phenyl)-4,5,6,6a-tetrahydro-3aH-pentalen-1-one (E19);
2-bromo-3-(3-bromo-4-hydroxy-phenyl)-4,5,6,6a-tetrahydro-3aH-pentalen-1-one (E20);
2-bromo-3-(3-chloro-4-hydroxy-phenyl)-4,5,6,6a-tetrahydro-3aH-pentalen-1-one (E21);
2-bromo-3-(3,5-dichloro-4-hydroxy-phenyl)-4,5,6,6a-tetrahydro-3aH-pentalen-1-one (E22);
2-bromo-3-(3-fluoro-4-hydroxy-phenyl)-4,5,6,6a-tetrahydro-3aH-pentalen-1-one (E23);
3-(4-hydroxy-3-methyl-phenyl)-4,5,6,6a-tetrahydro-3aH-pentalen-1-one (E24);
3-(2-fluoro-4-hydroxy-phenyl)-4,5,6,6a-tetrahydro-3aH-pentalen-1-one (E25);
2-bromo-3-(4-hydroxy-3-methyl-phenyl)-4,5,6,6a-tetrahydro-3aH-pentalen-1-one (E26);
2-bromo-3-(2-fluoro-4-hydroxy-phenyl)-4,5,6,6a-tetrahydro-3aH-pentalen-1-one (E27);
3-(3-fluoro-4-hydroxy-phenyl)-4,5,6,6a-tetrahydro-3aH-pentalen-1-one (E28);
2-bromo-3-(3-chloro-5-fluoro-4-hydroxy-phenyl)-4,5,6,6a-tetrahydro-3aH-pentalen-1-one (E29);
2-chloro-3-(3-chloro-5-fluoro-4-hydroxy-phenyl)-4,5,6,6a-tetrahydro-3aH-pentalen-1-one (E30);
3-(4-hydroxy-phenyl)-2-thiophen-2-yl-4,5,6,6a-tetrahydro-3aH-pentalen-1-one (E31);
3-(4-hydroxy-phenyl)-2-(3-methyl-thiophen-2-yl)-4,5,6,6a-tetrahydro-3aH-pentalen-1-one (E32);
3-(4-hydroxy-phenyl)-2-prop-1-ynyl-4,5,6,6a-tetrahydro-3aH-pentalen-1-one (E33);
2-ethynyl-3-(4-hydroxy-phenyl)-4,5,6,6a-tetrahydro-3aH-pentalen-1-one (E34);
2-[3-(4-hydroxy-phenyl)-1-oxo-1,3a,4,5,6,6a-hexahydro-pentalen-2-yl]-thiophene-3-carbonitrile (E35);
2-furan-2-yl-3-(4-hydroxy-phenyl)-4,5,6,6a-tetrahydro-3aH-pentalen-1-one (E36);
3-(4-hydroxy-phenyl)-2-vinyl-4,5,6,6a-tetrahydro-3aH-pentalen-1-one (E37);
3-(4-hydroxy-phenyl)-2-(2-methoxy-thiazol-4-yl)-4,5,6,6a-tetrahydro-3aH-pentalen-1-one (E38);
3-(4-hydroxy-phenyl)-2-thiazol-4-yl-4,5,6,6a-tetrahydro-3aH-pentalen-1-one (E39);
3-(4-hydroxy-phenyl)-2-thiazol-2-yl-4,5,6,6a-tetrahydro-3aH-pentalen-1-one (E40);
3-(4-hydroxy-phenyl)-2-(2-methyl-allyl)-4,5,6,6a-tetrahydro-3aH-pentalen-1-one (E41);
3-(4-hydroxy-phenyl)-2-(E)-propenyl)-4,5,6,6a-tetrahydro-3aH-pentalen-1-one (E42);
3-(4-hydroxy-phenyl)-2-(Z)-propenyl)-4,5,6,6a-tetrahydro-3aH-1-pentalen-1-one (E43);
3-(4-hydroxy-phenyl)-2-(3-methyl-but-2-enyl)-4,5,6,6a-tetrahydro-3aH-pentalen-1-one (E44);
2-acetyl-3-(4-hydroxy-phenyl)-4,5,6,6a-tetrahydro-3aH-pentalen-1-one (E45);
3-(4-hydroxy-phenyl)-2-thiophen-3-yl-4,5,6,6a-tetrahydro-3aH-pentalen-1-one (E46);
3-(4-hydroxy-phenyl)-2-isopropenyl-4,5,6,6a-tetrahydro-3aH-pentalen-1-one (E47);
3-(4-hydroxy-phenyl)-2-(1-methyl-1H-pyrrol-2-yl)-4,5,6,6a-tetrahydro-3aH-pentalen-1-one (E48); benzoic acid 4-(2-bromo-3-oxo-3,3a,4,5,6,6a-hexahydro-pentalen-1-yl)-phenyl ester (E49);
2-bromo-3-(4-dimethylamino-phenyl)-4,5,6,6a-tetrahydro-3aH-pentalen-1-one (E50);
2-bromo-3-(4-hydroxy-2,5-dimethyl-phenyl)-4,5,6,6a-tetrahydro-3aH-pentalen-1-one (E51);
3-(6-hydroxy-naphthalen-2-yl)-4,5,6,6a-tetrahydro-3aH-pentalen-1-one (E52);
2-bromo-3-(4-hydroxy-3,5-dimethyl-phenyl)-4,5,6,6a-tetrahydro-3aH-pentalen-1-one (E53);
2-bromo-3-(4-hydroxy-2-methyl-phenyl)-4,5,6,6a-tetrahydro-3aH-pentalen-1-one (E54); 3a-bromo-3-(3,5-difluoro-4-hydroxy-phenyl)-4,5,6,6a-tetrahydro-3aH-pentalen-1-one (E55);
2-(3,5-dimethyl-isoxazol-4-yl)-3-(4-hydroxy-phenyl)-4,5,6,6a-tetrahydro-3aH-pentalen-1-one (E56);
3-(4-amino-3-methyl-phenyl)-2-bromo-4,5,6,6a-tetrahydro-3aH-pentalen-1-one (E57);
3-(4-amino-phenyl)-4,5,6,6a-tetrahydro-3aH-pentalen-1-one (E58);
3-(4-amino-phenyl)-2-bromo-4,5,6,6a-tetrahydro-3aH-pentalen-1-one (E59);
3-(4-amino-3-bromo-phenyl)-2-bromo-4,5,6,6a-tetrahydro-3aH-pentalen-1-one (E60);
2-bromo-3-(1H-indazol-5-yl)-4,5,6,6a-tetrahydro-3aH-pentalen-1-one (E61);
3-(1H-indazol-5-yl)-1-oxo-1,3a,4,5,6,6a-hexahydro-pentalene-2-carbonitrile (E62);
3-(1H-indazol-5-yl)-4,5,6,6a-tetrahydro-3aH-pentalen-1-one (E63);
2-[3-(1H-indazol-5-yl)-1-oxo-1,3a,4,5,6,6a-hexahydro-pentalen-2-yl]-thiophene-3-carbonitrile (E64);
2-bromo-3-(4-isobutylamino-phenyl)-4,5,6,6a-tetrahydro-3aH-pentalen-1-one (E 65);
2-Bromo-3-(4-methylamino-phenyl)-4,5,6,6a-tetrahydro-3aH-pentalen-1-one (E66);
2-bromo-3-{4-[(furan-2-ylmethyl)-amino]-phenyl}-4,5,6,6a-tetrahydro-3aH-pentalen-1-one (E67);
2-Bromo-3-(4-pentylamino-phenyl)-4,5,6,6a-tetrahydro-3aH-pentalen-1-one (E68);
2-bromo-3-(4-hydroxy-phenyl)-5-methylene-4,5,6,6a-tetrahydro-3aH-pentalen-1-one (E69);
3-(4-hydroxy-phenyl)-5-methylene-4,5,6,6a-tetrahydro-3aH-pentalen-1-one (E 70);
2-benzyl-6-(4-hydroxy-phenyl)-2,3,3a,6a-tetrahydro-1H-cyclopenta[c]pyrrol-4-one (E 71);
(rac)-(3aS,5R,6aR)-5-bromo-3-(4-hydroxy-phenyl)-5-methyl-4,5,6,6a-tetrahydro-3aH-pentalen-1-one (E 72);
(rac)-(3aS,5R,6aR)-2,5-dibromo-3-(4-hydroxy-phenyl)-5-methyl-4,5,6,6a-tetrahydro-3aH-pentalen-1-one (E 73);
(rac)-(3aS,5S,6aR)-2,5-dibromo-3-(4-hydroxy-phenyl)-5-methyl-4,5,6,6a-tetrahydro-3aH-pentalen-1-one (E 74);
(rac)-(3aS,5S,6aR)-5-chloro-3-(4-hydroxy-phenyl)-5-methyl-4,5,6,6a-tetrahydro-3aH-pentalen-1-one (E 75);
(rac)-(3aS,5S,6aR)-2-bromo-5-chloro-3-(4-hydroxy-phenyl)-5-methyl-4,5,6,6a-tetrahydro-3aH-pentalen-1-one (E 76);
(rac)-(3aS,5R,6aR)-2-Bromo-5-chloro-3-(4-hydroxy-phenyl)-5-methyl-4,5,6,6a-tetrahydro-3aH-pentalen-1-one (E 77);
(rac)-(5R,6aS)-2,3a-dibromo-5-chloro-3-(4-hydroxy-phenyl)-5-methyl-4,5,6,6a-tetrahydro-3aH-pentalen-1-one (E 78);
(rac)-(5S,6aS)-3a-bromo-5-chloro-3-(4-hydroxy-phenyl)-5-methyl-4,5,6,6a-tetrahydro-3aH-pentalen-1-one (E 79);
2-bromo-3-(2,3-difluoro-4-hydroxy-phenyl)-4,5,6,6a-tetrahydro-3aH-pentalen-1-one (E80);
3-(2,3-difluoro-4-hydroxy-phenyl)-1-oxo-1,3a,4,5,6,6a-hexahydro-pentalene-2-carbonitrile (E81);
2-bromo-3-(2,5-difluoro-4-hydroxy-phenyl)-4,5,6,6a-tetrahydro-3aH-pentalen-1-one (E 82);
3-(3-fluoro-4-hydroxy-phenyl)-1-oxo-1,3a,4,5,6,6a-hexahydro-pentalene-2-carbonitrile (E83);
2-bromo-3-(3-fluoro-4-hydroxy-phenyl)-6a-methyl-4,5,6,6a-tetrahydro-3aH-pentalen-1-one (E84);
3-(3-fluoro-4-hydroxy-phenyl)-6a-methyl-1-oxo-1,3a,4,5,6,6a-hexahydro-pentalene-2-carbonitrile (E85);
2-bromo-3-(2,3-difluoro-4-hydroxy-phenyl)-6a-methyl-4,5,6,6a-tetrahydro-3aH-pentalen-1-one (E86);
2-bromo-3-(3,5-difluoro-4-hydroxy-phenyl)-4,5,6,6a-tetrahydro-3aH-pentalen-1-one (E87);
3-(2,3-difluoro-4-hydroxy-phenyl)-6a-methyl-1-oxo-1,3a,4,5,6,6a-hexahydro-pentalene-2-carbonitrile (E88);
3-(3-chloro-5-fluoro-4-hydroxy-phenyl)-1-oxo-1,3a,4,5,6,6a-hexahydro-pentalene-2-carbonitrile (E89);
3-(3,5-difluoro-4-hydroxy-phenyl)-3a-hydroxy-1-oxo-1,3a,4,5,6,6a-hexahydro-pentalene-2-carbonitrile (E90);
3-(3,5-difluoro-4-hydroxy-phenyl)-4,5,6,6a-tetrahydro-3aH-pentalen-1-one (E91);
3-(3,5-difluoro-4-hydroxy-phenyl)-1-oxo-1,3a,4,5,6,6a-hexahydro-pentalene-2-carbonitrile (E92);
3-(2,5-difluoro-4-hydroxy-phenyl)-1-oxo-1,3a,4,5,6,6a-hexahydro-pentalene-2-carbonitrile (E93);
2-bromo-3-(2,5-difluoro-4-hydroxy-phenyl)-6a-methyl-4,5,6,6a-tetrahydro-3aH-pentalen-1-one (E94);
3-(2,5-difluoro-4-hydroxy-phenyl)-6a-methyl-1-oxo-1,3a,4,5,6,6a-hexahydro-pentalene-2-carbonitrile (E95);
2,6a-dibromo-3-(2,3-difluoro-4-hydroxy-phenyl)-4,5,6,6a-tetrahydro-3aH-pentalen-1-one (E96);
2-bromo-3-(3,5-difluoro-4-hydroxy-phenyl)-4,5,6,6a-tetrahydro-3aH-pentalen-1-one oxime (E97);
3-(3,5-Difluoro-4-hydroxy-phenyl)-1-hydroxyimino-1,3a,4,5,6,6a-hexahydro-pentalene-2-carbonitrile (E98);
2-bromo-3-(3,5-difluoro-4-hydroxy-phenyl)-6a-methyl-4,5,6,6a-tetrahydro-3aH-pentalen-1-one (E99);
3-(3,5-difluoro-4-hydroxy-phenyl)-6a-methyl-1-oxo-1,3a,4,5,6,6a-hexahydro-pentalene-2-carbonitrile (E100);
2-bromo-3-(3,5-difluoro-4-hydroxy-phenyl)-6a-methyl-4,5,6,6a-tetrahydro-3aH-pentalen-1-one oxime (E101);
3-(3-chloro-4-hydroxy-phenyl)-1-oxo-1,3a,4,5,6,6a-hexahydro-pentalene-2-carbonitrile (E102);
2-bromo-3-(3-chloro-5-fluoro-4-hydroxy-phenyl)-6a-methyl-4,5,6,6a-tetrahydro-3aH-pentalen-1-one (E103);
3-(3-chloro-5-fluoro-4-hydroxy-phenyl)-6a-methyl-1-oxo-1,3a,4,5,6,6a-hexahydro-pentalene-2-carbonitrile (E104);
2-bromo-3-(2-chloro-4-hydroxy-phenyl)-4,5,6,6a-tetrahydro-3aH-pentalen-1-one (E105);
3-(2-fluoro-4-hydroxy-phenyl)-1-oxo-1,3a,4,5,6,6a-hexahydro-pentalene-2-carbonitrile (E106);
2-bromo-3-(5-chloro-2,3-difluoro-4-hydroxy-phenyl)-4,5,6,6a-tetrahydro-3aH-pentalen-1-one (E107);
2-bromo-3-(2,3-dichloro-4-hydroxy-phenyl)-4,5,6,6a-tetrahydro-3aH-pentalen-1-one (E108);
3-(2,3-dichloro-4-hydroxy-phenyl)-4,5,6,6a-tetrahydro-3aH-pentalen-1-one (E109);
3-(5-chloro-2,3-difluoro-4-hydroxy-phenyl)-1-oxo-1,3a,4,5,6,6a-hexahydro-pentalene-2-carbonitrile (E110);
3-(2-chloro-4-hydroxy-phenyl)-1-oxo-1,3a,4,5,6,6a-hexahydro-pentalene-2-carbonitrile (E111);
2-bromo-3-(2-chloro-3-fluoro-4-hydroxy-phenyl)-4,5,6,6a-tetrahydro-3aH-pentalen-1-one (E112);
2-bromo-3-(5-chloro-2-fluoro-4-hydroxy-phenyl)-1-oxo-1,3a,4,5,6,6a-hexahydro-pentalene-2-carbonitrile (E113);
2-bromo-3-(5-bromo-2-chloro-3-fluoro-4-hydroxy-phenyl)-4,5,6,6a-tetrahydro-3aH-pentalen-1-one (E114);
3-(5-chloro-2-fluoro-4-hydroxy-phenyl)-1-oxo-1,3a,4,5,6,6a-hexahydro-pentalene-2-carbonitrile (E115);
3-(2-chloro-3-fluoro-4-hydroxy-phenyl)-4,5,6,6a-tetrahydro-3aH-pentalen-1-one (E116);
3-(2-chloro-3-fluoro-4-hydroxy-phenyl)-1-oxo-1,3a,4,5,6,6a-hexahydro-pentalene-2-carbonitrile E117);
2-bromo-3-(2,6-difluoro-4-hydroxy-phenyl)-4,5,6,6a-tetrahydro-3aH-pentalen-1-one (E118);
3-(2,6-difluoro-4-hydroxy-phenyl)-1-oxo-1,3a,4,5,6,6a-hexahydro-pentalene-2-carbonitrile (E119);
2-bromo-3-(3-chloro-2-fluoro-4-hydroxy-phenyl)-4,5,6,6a-tetrahydro-3aH-pentalen-1-one (E120);
(3aS,6aR)-3-(2,3-difluoro-4-hydroxy-phenyl)-1-oxo-1,3a,4,5,6,6a-hexahydro-pentalene-2-carbonitrile (E121);
(3aR,6a S)-3-(2,3-difluoro-4-hydroxy-phenyl)-1-oxo-1,3a,4,5,6,6a-hexahydro-pentalene-2-carbonitrile (E122);
3-(3-chloro-2-fluoro-4-hydroxy-phenyl)-1-oxo-1,3a,4,5,6,6a-hexahydro-pentalene-2-carbonitrile (E123);
2-bromo-3-(2,3,5-trifluoro-4-hydroxy-phenyl)-4,5,6,6a-tetrahydro-3aH-pentalen-1-one (E124);
2-bromo-3-(3-chloro-2,5-difluoro-4-hydroxy-phenyl)-4,5,6,6a-tetrahydro-3aH-pentalen-1-one (E125);
3-(3-chloro-2,5-difluoro-4-hydroxy-phenyl)-1-oxo-1,3a,4,5,6,6a-hexahydro-pentalene-2-carbonitrile (E126);
2-bromo-3-(2,3,6-trifluoro-4-hydroxy-phenyl)-4,5,6,6a-tetrahydro-3aH-pentalen-1-one (E127);
2-bromo-3-(4-hydroxy-phenyl)-6a-propyl-4,5,6,6a-tetrahydro-3aH-pentalen-1-one (E128);
3-(3,5-difluoro-4-hydroxy-phenyl)-2-ethynyl-4,5,6,6a-tetrahydro-3aH-pentalen-1-one (E129);
3-(2,3-difluoro-4-hydroxy-phenyl)-2-isopropenyl-4,5,6,6a-tetrahydro-3aH-pentalen-1-one (E130);
3-(3,5-difluoro-4-hydroxy-phenyl)-2-isopropenyl-4,5,6,6a-tetrahydro-3aH-pentalen-1-one (E131);
2-Bromo-3-(2,3-difluoro-4-hydroxy-phenyl)-4,5,6,7,8,8a-hexahydro-3aH-azulen-1-one (E132);
   or a pharmaceutically acceptable ester, amide, solvate or salt thereof, including a salt of such an ester or amide, and a solvate of such an ester, amide or salt.

The following compounds were also synthesised:
2-Bromo-3-(4-diallylamino-phenyl)-4,5,6,6a-tetrahydro-3aH-pentalen-1-one (C133);
2-Bromo-3-(4-diallylamino-3-methyl-phenyl)-4,5,6,6a-tetrahydro-3aH-pentalen-1-one (C134);
3-(4-Allylamino-3-methyl-phenyl)-2-bromo-4,5,6,6a-tetrahydro-3aH-pentalen-1-one (C135);
2-Chloro-3-(3-chloro-1H-indazol-5-yl)-4,5,6,6a-tetrahydro-3aH-pentalen-1-one (C136);
2-Bromo-3-(3-chloro-1H-indazol-5-yl)-4,5,6,6a-tetrahydro-3aH-pentalen-1-one (C137);
3-(3-Bromo-1H-indazol-5-yl)-4,5,6,6a-tetrahydro-3aH-pentalen-1-one (C138);
2-Bromo-3-(3-bromo-1H-indazol-5-yl)-4,5,6,6a-tetrahydro-3aH-pentalen-1-one (C139);
Ethanesulfonic acid [4-(2-bromo-3-oxo-3,3a,4,5,6,6a-hexahydro-pentalen-1-yl)-phenyl]-amide (C140);
N-[4-(2-Bromo-3-oxo-3,3a,4,5,6,6a-hexahydro-pentalen-1-yl)-phenyl]-methanesulfonamide (C141);
N-[4-(2-Bromo-3-oxo-3,3a,4,5,6,6a-hexahydro-pentalen-1-yl)-phenyl]-4-fluoro-benzenesulfonamide (C142);
N-[4-(2-Bromo-3-oxo-3,3a,4,5,6,6a-hexahydro-pentalen-1-yl)-phenyl]-benzenesulfonamide (C143);
Propane-1-sulfonic acid [4-(2-bromo-3-oxo-3,3a,4,5,6,6a-hexahydro-pentalen-1-yl)-phenyl]-amide (C144);
N-[4-(2-Bromo-3-oxo-3,3a,4,5,6,6a-hexahydro-pentalen-1-yl)-phenyl]-propionamide (C145);
N-[4-(2-Bromo-3-oxo-3,3a,4,5,6,6a-hexahydro-pentalen-1-yl)-phenyl]-benzamide (C146);
N-[4-(2-Bromo-3-oxo-3,3a,4,5,6,6a-hexahydro-pentalen-1-yl)-phenyl]-butyramide (C147); and
(rac)-(3aS,5R,6aR)-5-Hydroxy-3-(4-hydroxy-phenyl)-5-methyl-4,5,6,6a-tetrahydro-3aH-pentalen-1-one (C148).

Compounds 133-148 find use as medicaments for the treatment of diseases related to the estrogen receptor.

The compound names given above were generated in accordance with IUPAC by the ACD Labs 8.0/name program, version 8.05 and/or with ISIS DRAW Autonom 2000.

Salts and solvates of compounds of formula (I) which are suitable for use in medicine are those wherein a counterion or associated solvent is pharmaceutically acceptable. However, salts and solvates having non-pharmaceutically acceptable counterions or associated solvents are within the scope of the present invention, for example, for use as intermediates in the preparation of the compounds of formula (I) and their pharmaceutically acceptable salts, solvates and physiologically functional derivatives. By the term “physiologically functional derivative” is meant a chemical derivative of a compound of formula (I) having the same physiological function as the free compound of formula (I), for example, by being convertible in the body thereto. According to the present invention, examples of physiologically functional derivatives include esters, amides, and carbamates; preferably esters and amides.

Suitable salts according to the invention include those formed with organic or inorganic acids or bases. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Other acids such as oxalic, while not in themselves pharmaceutically acceptable, may be useful as intermediates in obtaining the compounds of the invention and their pharmaceutical acceptable acid addition salts. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may furthermore be formed.

Pharmaceutically acceptable esters and amides of the compounds of formula (I) may have an appropriate group, for example an acid group, converted to a C₁₋₆ alkyl, phenyl, benzyl, C₅₋₈ heterocyclyl, or amino acid ester or amide. Pharmaceutically acceptable esters of the compounds of formula (I) may have an appropriate group, for example a hydroxy group, converted to a C₁₋₆ alkyl, phenyl, benzyl or C₅₋₈ heterocyclyl ester. Pharmaceutically acceptable amides and carbamates of the compounds of formula (I) may have an appropriate group, for example an amino group, converted to a C₁₋₆ alkyl, phenyl, benzyl, C₅₋₈ heterocyclyl, or amino acid ester or amide, or carbamate.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”.

A compound which, upon administration to the recipient, is capable of being converted into a compound of formula (I) as described above, or an active metabolite or residue thereof, is known as a “prodrug”. A prodrug may, for example, be converted within the body, e.g. by hydrolysis in the blood, into its active form that has medical effects. Pharmaceutical acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987, which are incorporated herein by reference.

The following definitions apply to the terms as used throughout this specification, unless otherwise limited in specific instances.

As used herein, the term “alkyl” means both straight and branched chain saturated hydrocarbon groups. Examples of alkyl groups include methyl, ethyl, n-propyl, iso-propyl, n-butyl, t-butyl, i-butyl, sec-butyl, pentyl, hexyl, heptyl, octyl, nonyl, and decyl groups. Among unbranched alkyl groups, there are preferred methyl, ethyl, n-propyl, iso-propyl, n-butyl groups. Among branched alkyl groups, there may be mentioned t-butyl, i-butyl, 1-ethylpropyl, 1-ethylbutyl, and 1-ethylpentyl groups.

As used herein, the term “alkoxy” means the group O-alkyl, where “alkyl” is used as described above. Examples of alkoxy groups include methoxy and ethoxy groups. Other examples include propoxy and butoxy.

As used herein, the term “alkenyl” means both straight and branched chain unsaturated hydrocarbon groups with at least one carbon carbon double bond. Up to 5 carbon carbon double bonds may, for example, be present. Examples of alkenyl groups include ethenyl, propenyl, butenyl, pentenyl, hexenyl, heptenyl, octenyl, nonenyl, decenyl and dodecenyl. Preferred alkynyl groups include ethenyl, 1-propenyl and 2-propenyl.

As used herein, the term “alkynyl” means both straight and branched chain unsaturated hydrocarbon groups with at least one carbon carbon triple bond. Up to 5 carbon carbon triple bonds may, for example, be present. Examples of alkynyl groups include ethynyl, propynyl, butynyl, pentynyl, hexynyl, heptynyl, octynyl, nonynyl, decynyl and dodecynyl. Preferred alkenyl groups include ethynyl 1-propynyl and 2-propynyl.

As used herein, the term “cycloalkyl” means a saturated group in a ring system. The cycloalkyl group can be monocyclic or bicyclic. A bicyclic group may, for example, be fused or bridged. Examples of monocyclic cycloalkyl groups include cyclopropyl, cyclobutyl and cyclopentyl. Other examples of monocyclic cycloalkyl groups are cyclohexyl, cycloheptyl and cyclooctyl. Examples of bicyclic cycloalkyl groups include bicyclo [2.2.1]hept-2-yl. Preferably, the cycloalkyl group is monocyclic.

As used herein, the term “aryl” means a monocyclic or bicyclic aromatic carbocyclic group. Examples of aryl groups include phenyl and naphthyl. A naphthyl group may be attached through the 1 or the 2 position. In a bicyclic aromatic group, one of the rings may, for example, be partially saturated. Examples of such groups include indanyl and tetrahydronaphthyl. Specifically, the term C₅₋₁₀ aryl is used herein to mean a group comprising from 5 to 10 carbon atoms in a monocyclic or bicyclic aromatic group. A particularly preferred C₅₋₁₀ aryl group is phenyl.

As used herein, the term “halogen” means fluorine, chlorine, bromine or iodine. Fluorine, chlorine and bromine are particularly preferred. In some embodiments, fluorine is especially preferred. In alternative embodiments, chlorine or bromine are especially preferred.

As used herein, the term “haloalkyl” means an alkyl group having a halogen substituent, the terms “alkyl” and “halogen” being understood to have the meanings outlined above. Similarly, the term “dihaloalkyl” means an alkyl group having two halogen substituents and the term “trihaloalkyl” means an alkyl group having three halogen substituents. Examples of haloalkyl groups include fluoromethyl, chloromethyl, bromomethyl, fluoromethyl, fluoropropyl and fluorobutyl groups; examples of dihaloalkyl groups include difluoromethyl and difluoroethyl groups; examples of trihaloalkyl groups include trifluoromethyl and trifluoroethyl groups.

As used herein, the term “heterocyclyl” means an aromatic (“heteroaryl”) or a non-aromatic (“heterocycloalkyl”) cyclic group of carbon atoms wherein from one to three of the carbon atoms is/are replaced by one or more heteroatoms independently selected from nitrogen, oxygen or sulfur. A heterocyclyl group may, for example, be monocyclic or bicyclic. In a bicyclic heterocyclyl group there may be one or more heteroatoms in each ring, or only in one of the rings. A heteroatom is preferably O or N. Heterocyclyl groups containing a suitable nitrogen atom include the corresponding N-oxides. Examples of monocyclic heterocycloalkyl rings include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl.

Specifically, the term C₅₋₁₀ heterocyclyl is used herein to mean a group comprising from 5 to 10 carbon atoms in a monocyclic or bicyclic aromatic (“heteroaryl”) or non-aromatic (“heterocycloalkyl”) cyclic group wherein from one to three of the carbon atoms is/are replaced by one or more heteroatoms independently selected from nitrogen, oxygen or sulfur. Preferred heterocyclyl groups are C₅₋₈ heterocyclyl groups, particularly C₅₋₈ heterocyclyl groups, especially C₅ heterocyclyl groups. More specifically, the term C₅ heterocyclyl is used herein to mean a 5-membered aromatic (“heteroaryl”) or non-aromatic (“heterocycloalkyl”) cyclic group comprising from one to three heteroatoms independently selected from nitrogen, oxygen or sulfur, the remainder of the 5-membered ring atoms being carbon atoms. Examples of C₅ heterocyclyl groups include furanyl, thiophenyl, pyrrolyl, imidazolyl, oxazolyl, thiazolyl, and their partially or fully saturated analogues such as dihydrofuranyl and tetrahydrofuranyl.

Examples of bicyclic heterocyclic rings in which one of the rings is non-aromatic include dihydrobenzofuranyl, indanyl, indolinyl, isoindolinyl, tetrahydroisoquinolinyl, tetrahydroquinolyl and benzoazepanyl.

Examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; examples of bicyclic heteroaryl groups include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazoly[4,5-b]pyridiyl, pyridopyrimidinyl, isoquinolinyl and benzodroxazole.

Examples of preferred heterocyclyl groups include piperidinyl, tetrahydrofuranyl, tetrahydropyranyl, pyridyl, pyrimidyl and indolyl. Preferred heterocyclyl groups also include thiophenyl, thiazolyl, furanyl, pyrazolyl, pyrrolyl and imidazolyl.

As used herein the term “cycloalkylalkyl” means a group cycloalkyl-alkyl-attached through the alkyl group, “cycloalkyl” and “alkyl” being understood to have the meanings outlined above.

As mentioned above, the compounds of the invention have activity as estrogen receptor ligands. The compounds of the invention have activity as estrogen receptor modulators, and may be agonists, partial agonists, antagonists, or partial antagonists of the estrogen receptor. Particularly preferred compounds of the invention have activity as an agonist or a partial agonist of ERβ. Preferred compounds of this type are selective agonists of the estrogen receptor-beta (ERβ).

The compounds of the invention may thus be used in the treatment of diseases or disorders associated with estrogen receptor activity. In particular, the compounds of the invention that are agonists or partial agonists of the estrogen receptor may be used in the treatment of diseases or disorders for which selective agonists or partial agonists of the estrogen receptor are indicated. The compounds of the invention that are antagonists or partial antagonists of the estrogen receptor may be used in the treatment of diseases or disorders for which selective antagonists or partial antagonists of the estrogen receptor are indicated.

Clinical conditions for which an agonist or partial agonist is indicated include, but are not limited to, bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration, and lung, colon, breast, uterus, and prostate cancer, and/or disorders related to estrogen functioning.

The compounds of the invention find particular application in the treatment or prophylaxis of the following: bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration, and lung, colon, breast, uterus, and prostate cancer, and/or disorders related to estrogen functioning.

The invention also provides a method for the treatment or prophylaxis of a condition in a mammal mediated by an estrogen receptor, which comprises administering to the mammal a therapeutically effective amount of a compound of formula (I) as defined above or a pharmaceutically acceptable ester, amide, solvate or salt thereof, including a salt of such an ester or amide, and a solvate of such an ester, amide or salt. Clinical conditions mediated by an estrogen receptor that may be treated by the method of the invention are those described above.

The invention also provides the use of a compound of formula (I) as defined above or a pharmaceutically acceptable ester, amide, solvate or salt thereof, including a salt of such an ester or amide, and a solvate of such an ester, amide or salt, for the manufacture of a medicament for the treatment or prophylaxis of a condition mediated by an estrogen receptor. Clinical conditions mediated by an estrogen receptor that may be treated by the method of the invention are those described above.

Hereinafter, the term “active ingredient” means a compound of formula (I) as defined above, or a pharmaceutically acceptable ester, amide, solvate or salt thereof, including a salt of such an ester or amide, and a solvate of such an ester, amide or salt.

The amount of active ingredient which is required to achieve a therapeutic effect will, of course, vary with the particular compound, the route of administration, the subject under treatment, including the type, species, age, weight, sex, and medical condition of the subject and the renal and hepatic function of the subject, and the particular disorder or disease being treated, as well as its severity. An ordinarily skilled physician, veterinarian or clinician can readily determine and prescribe the effective amount of the drug required to prevent, counter or arrest the progress of the condition.

Oral dosages of the present invention, when used for the indicated effects, will range between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day, for adult humans. For oral administration, the compositions are preferably provided in the form of tablets or other forms of presentation provided in discrete units containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or the total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches well known to those of ordinary skill in the art. To be administered in the form of a transdermal delivery system, the dosage administration will, of course, be continuous rather than intermittent throughout the dosage regimen.

While it is possible for the active ingredient to be administered alone, it is preferable for it to be present in a pharmaceutical formulation or composition. Accordingly, the invention provides a pharmaceutical formulation comprising a compound of formula (I) as defined above or a pharmaceutically acceptable ester, amide, solvate or salt thereof, including a salt of such an ester or amide, and a solvate of such an ester, amide or salt, and a pharmaceutically acceptable diluent, excipient or carrier (collectively referred to herein as “carrier” materials). Pharmaceutical compositions of the invention may take the form of a pharmaceutical formulation as described below.

The pharmaceutical formulations according to the invention include those suitable for oral, parenteral (including subcutaneous, intradermal, intramuscular, intravenous [bolus or infusion], and intraarticular), inhalation (including fine particle dusts or mists which may be generated by means of various types of metered does pressurized aerosols), nebulizers or insufflators, rectal, intraperitoneal and topical (including dermal, buccal, sublingual, and intraocular) administration, although the most suitable route may depend upon, for example, the condition and disorder of the recipient.

The formulations may conveniently be presented in unit dosage form and may be prepared by any of the methods well known in the art of pharmacy. All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets, pills or tablets each containing a predetermined amount of the active ingredient; as a powder or granules; as a solution or a suspension in an aqueous liquid or a non-aqueous liquid, for example as elixirs, tinctures, suspensions or syrups; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein. The present compounds can, for example, be administered in a form suitable for immediate release or extended release. Immediate release or extended release can be achieved by the use of suitable pharmaceutical compositions comprising the present compounds, or, particularly in the case of extended release, by the use of devices such as subcutaneous implants or osmotic pumps. The present compounds can also be administered liposomally.

Exemplary compositions for oral administration include suspensions which can contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which can contain, for example, microcrystalline cellulose, dicalcium phosphate, starch, magnesium stearate, calcium sulfate, sorbitol, glucose and/or lactose and/or other excipients, binders, extenders, disintegrants, diluents and lubricants such as those known in the art. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Disintegrators include without limitation starch, methylcellulose, agar, bentonite, xanthan gum and the like. The compounds of formula (I) can also be delivered through the oral cavity by sublingual and/or buccal administration. Molded tablets, compressed tablets or freeze-dried tablets are exemplary forms which may be used. Exemplary compositions include those formulating the present compound(s) with fast dissolving diluents such as mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. Lubricants used in these dosage forms include sodium oleate, sodium stearate, magnesium stearate, sodium benzoate, sodium acetate, sodium chloride and the like. For oral administration in liquid form, the oral drug components can be combined with any oral, non-toxic, pharmaceutically acceptable inert carrier such as ethanol, glycerol, water, and the like.

The compounds of the present invention can also be administered in the form of liposome delivery systems, such as small unilamellar vesicles, large unilamellar vesicles and multilamellar vesicles. Liposomes can be formed from a variety of phospholipids, 1,2-dipalmitoyl-phosphatidylcholine, phosphatidyl ethanolamine (cephaline), or phosphatidylcholine (lecithin).

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Exemplary compositions for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Formulations for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Formulations for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage formulations are those containing an effective dose, as hereinbefore recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavouring agents.

Whilst a compound of the invention may be used as the sole active ingredient in a medicament, it is also possible for the compound to be used in combination with one or more further active agents. Such further active agents may be further compounds according to the invention, or they may be different therapeutic agents, for example an antidepressant, an anxiolytic, an anti-psychotic, or an agent useful in the prevention or treatment of osteoporosis or other pharmaceutically active material. For example, the compounds of the instant invention may be effectively administered in combination with effective amounts of other agents such as an antidepressant, an anxiolytic, an anti-psychotic, an organic bisphosphonate or a cathepsin K inhibitor. Nonlimiting examples of antidepressants include noradrenaline reuptake inhibitors (NRI), selective serotonin reuptake inhibitors, monoamine oxidase inhibitors, tricyclic antidepressants (TCA), dopamine reuptake inhibitors (DRI), opioids, selective seretonic reuptake enhancers, tetracyclic antidepressants, reversible inhibitors of monoamine oxidase, melatonin agonists, serotonin and noradrenaline reuptake inhibitors (SNRI), corticotropin releasing factor antagonists, α-adrenoreceptor antagonists, 5HT1α receptor agonists and antagonists, lithium and atypical anti-psychotics. Examples of antidepressants of the SSRI class include Fluoxetine and Sertraline; examples of antidepressants of the SNRI class Venlafaxine, Citalopram, Paroxetine, Escitalopram, Fluvoxamine; examples of antidepressants of the SNRI class include Duloxetine; examples of antidepressants of the DRI and NRI classes include Bupropion; examples of antidepressants of the TCA class include Amitriptyline and Dothiepin (Dosulepin). Examples of atypical antipsychotics include: Clozapine, Olanzapine, Risperidone, Quetiapine, Ziprasidone and Dopamine partial agonists. Nonlimiting examples of anxiolytics include benzodiazepines and non-benzodiazapines. Examples of benzodiazapines include lorazepam, alprazolam, and diazepam. Examples of non-benzodiazapines include Buspirone (Buspar®), barbiturates and meprobamate. One or more of those further anti-depressants may be used in combination.

Nonlimiting examples of said organic bisphosphonates include adendronate, clodronate, etidronate, ibandronate, incadronate, minodronate, neridronate, risedronate, piridronate, pamidronate, tiludronate, zoledronate, pharmaceutically acceptable salts or esters thereof, and mixtures thereof. Preferred organic biphosphonates include alendronate and pharmaceutically acceptable salts and mixtures thereof. Most preferred is alendronate monosodium trihydrate.

The precise dosage of the bisphosphonate will vary with the dosing schedule, the oral potency of the particular bisphosphonate chosen, the age, size, sex and condition of the mammal or human, the nature and severity of the disorder to be treated, and other relevant medical and physical factors. Thus, a precise pharmaceutically effective amount cannot be specified in advance and can be readily determined by the caregiver or clinician. An appropriate amount can be determined by routine experimentation from animal models and human clinical studies. Generally, an appropriate amount of bisphosphonate is chosen to obtain a bone resorption inhibiting effect, i.e. a bone resorption inhibiting amount of the bisphonsphonate is administered. For humans, an effective oral dose of bisphosphonate is typically from about 1.5 to about 6000 μg/kg of body weight and preferably about 10 to about 2000 μg/kg of body weight.

For human oral compositions comprising alendronate, pharmaceutically acceptable salts thereof, or pharmaceutically acceptable derivatives thereof, a unit dosage typically comprises from about 8.75 mg to about 140 mg of the alendronate compound, on an alendronic acid active weight basis, i.e. on the basis of the corresponding acid.

The compounds of the present invention can be used in combination with other agents useful for treating estrogen-mediated conditions. The individual components of such combinations can be administered separately at different times during the course of therapy or concurrently in divided or single combination forms. The present invention is therefore to be understood as embracing all such regimes of simultaneous or alternating treatment and the term “administering” is to be interpreted accordingly. It will be understood that the scope of combinations of the compounds of this invention with other agents useful for treating estrogen-mediated conditions includes in principle any combination with any pharmaceutical composition useful for treating disorders related to estrogen functioning.

The above other therapeutic agents, when employed in combination with the compounds of the present invention, may be used, for example, in those amounts indicated in the Physicians' Desk Reference (PDR) or as otherwise determined by one of ordinary skill in the art.

Where the compounds of the invention are utilized in combination with one or more other therapeutic agent(s), either concurrently or sequentially, the following combination ratios and dosage ranges are preferred:

When combined with an antidepressant, an anxiolytic, an anti-psychotic, an organic bisphosphonate or a cathepsin K inhibitor, the compounds of formula (I) may be employed in a weight ratio to the additional agent within the range from about 10:1 to about 1:10.

The compounds of formula (I) as described above also find use, optionally in labelled form, as a diagnostic agent for the diagnosis of conditions associated with malfunction of the estrogen receptor. For example, such a compound may be radioactively labelled.

The compounds of formula (I) as described above, optionally in labelled form, also find use as a reference compound in methods of discovering other agonists, partial agonists, antagonists or partial antagonists of the estrogen receptor. Thus, the invention provides a method of discovering a ligand of the estrogen receptor which comprises use of a compound of the invention or a compound of the invention in labelled form, as a reference compound. For example, such a method may involve a competitive binding experiment in which binding of a compound of formula (I) to the estrogen receptor is reduced by the presence of a further compound which has estrogen receptor-binding characteristics, for example stronger estrogen receptor-binding characteristics than the compound of formula (I) in question.

Numerous synthetic routes to the compounds of the present invention can be devised by any person skilled in the art and the possible synthetic routes described below do not limit the invention.

Accordingly, the invention provides a method for preparing a compound of formula (I) in accordance with the invention as described above wherein R¹¹ is halogen, comprising a step of reacting a compound of formula (II)

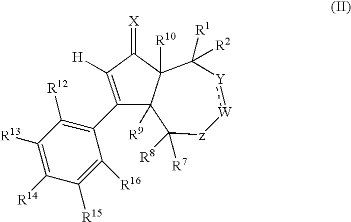

wherein X, Y, W, Z, R¹, R², R³, R⁴, R⁵, R⁶, R⁷, R⁸, R³⁰, R⁴⁰, R⁹, R¹⁰, R¹², R¹³, R¹⁴, R¹⁵ and R¹⁶ are as defined above
with a suitable halogenating reagent, for example N-halosuccinimide, and optionally followed by interconversion to another compound of formula (I) in accordance with the invention as described above.

The reaction mixture is stirred until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

Compounds of formula (II) wherein R¹⁰ is hydrogen and R¹¹ is hydrogen may be prepared by reacting a compound of formula (III)

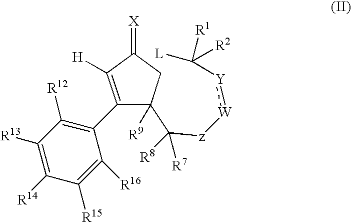

wherein X, Y, W, Z, R¹, R², R³, R⁴, R⁵, R⁶, R⁷, R⁸, R³⁰, R⁴⁰, R⁹, R¹², R¹³, R¹⁴, R¹⁵ and R¹⁶ are as defined above, and L is a suitable leaving group
in the presence of a suitable base, and optionally followed by interconversion to another compound of formula (I) in accordance with the invention as described above.

Suitable bases include alkylamines, for example triethylamine, KH or KO^{t}Bu. Other bases may be employed, as is known by the person skilled in the art. Suitable leaving groups L include halogens, for example a chloride. Alternatively the leaving group L may be a trimethylsilyl group, which may optionally be introduced during the reaction, for example by displacement of another leaving group, such as a halogen, for example a chloride, using trimethylsilyl chloride. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

The invention also provides a method for preparing a compound of formula (I) in accordance with the invention as described above wherein X is O, comprising a step of reacting a compound of formula (IV)

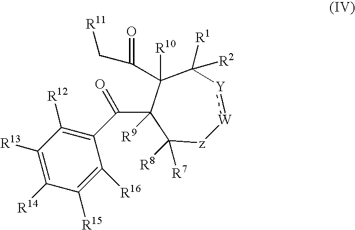

wherein Y, W, Z, R¹, R², R³, R⁴, R⁵, R⁶, R⁷, R⁸, R³⁰, R⁴⁰, R⁹, R¹⁰, R¹¹, R¹², R¹³, R¹⁴, R¹⁵ and R¹⁶ are as defined above
with a suitable reagent and optionally followed by interconversion to another compound of formula (I) in accordance with the invention as described above.

Suitable reagents include nucleophilic bases, for example DBU. The reaction mixture is stirred at room temperature, or heated until the starting materials have been consumed. The reaction may be carried out with protecting groups present and those protecting groups may be removed after the reaction. Suitable protecting groups are known to the person skilled in the art (see T. W. Greene, “Protective Groups in Organic Synthesis”, 3^{rd} Edition, New York, 1999).

### EXAMPLES

The novel compounds of the present invention can be prepared according to the procedure of the following Schemes and examples, using appropriate materials and are further exemplified by the following specific examples. The compounds illustrated in the examples are not, however, to be construed as forming the only genus that is considered as the invention. The following examples further illustrate details for the preparation of the compounds of the present invention. Those skilled in the art will readily understand that known variation of the conditions and processes of the following preparative procedures can be used to prepare these compounds.

### General Experimental Conditions

The compounds of the present invention of formula (I) are prepared according to the general methods outlined in Schemes 1-6, and according to the related methods described. All temperatures are degrees Celsius unless otherwise noted. The following abbreviations, reagents, expressions or equipment, which are amongst those used in the descriptions below, are explained as follows: 20-25° C. (room temperature, r.t.), molar equivalent (eq.), dimethyl formamide, (DMF) dichloromethane (DCM), ethyl acetate (EtOAc), tetrahydrofuran (THF), lithium diisopropylamide (LDA), pyridinium chlorochromate (PCC), preparative liquid chromatography with a C8 stationary phase and ammonium acetate acetonitrile-water buffer as mobile phase (PHPLC), electrospray mass spectroscopy (ES/MS).

### Examples 1-18

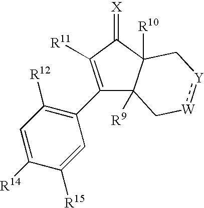

| Example | X | W | Y | R⁹ | R¹⁰ | R¹¹ | R¹² | R¹⁴ | R¹⁵ |
|---|---|---|---|---|---|---|---|---|---|
| 1 | O | bond | CH₂ | H | H | phenyl | H | OH | H |
| 2* | O | bond | CH₂ | H | H | Br | H | OH | H |
| 3^{†} | O | bond | CH₂ | H | H | Br | H | OH | H |
| 4^{‡} | O | bond | CH₂ | H | H | Br | H | OH | H |
| 5 | O | bond | CHMe | H | H | Br | H | OH | H |
| 6 | O | bond | CHEt | H | H | Br | H | OH | H |
| 7 | O | bond | CH₂ | H | H | Cl | H | OH | H |
| 8 | O | bond | CH₂ | H | H | CN | H | OH | H |
| 9 | O | bond | CH₂ | H | H | CF₃ | H | OH | H |
| 10 | O | bond | CH₂ | H | H | cyclopropyl | H | OH | H |
| 11 | O | bond | CH₂ | H | H | Br | H | OC(O)^{t}Bu | H |
| 12 | O | bond | CH₂ | H | F | Br | H | OH | H |
| 13 | O | bond | CH₂ | H | Me | Br | H | OH | H |
| 14** | O | CH | CH | H | H | H | H | OH | H |
| 15 | O | CH₂ | CH₂ | H | H | H | H | OH | H |
| 16 | O | CH₂ | CH₂ | H | H | Br | H | OH | H |
| 17 | NOH | bond | CH₂ | H | H | Br | H | OH | H |
| 18 | O | bond | CH₂ | H | H | Br | H | NHC(O)Me | H |

*Racemic compound
^{†}(3aR,6aS)-enantiomer
^{‡}(3aS,6aR)-enantiomer
**The bond between Y and W is a double bond in Example 14

### Example 1

### 3-(4-Hydroxy-phenyl)-2-phenyl-4,5,6,6a-tetrahydro-3aH-pentalen-1-one (E1)

The title compound was synthesized according to the method outlined in scheme 1.

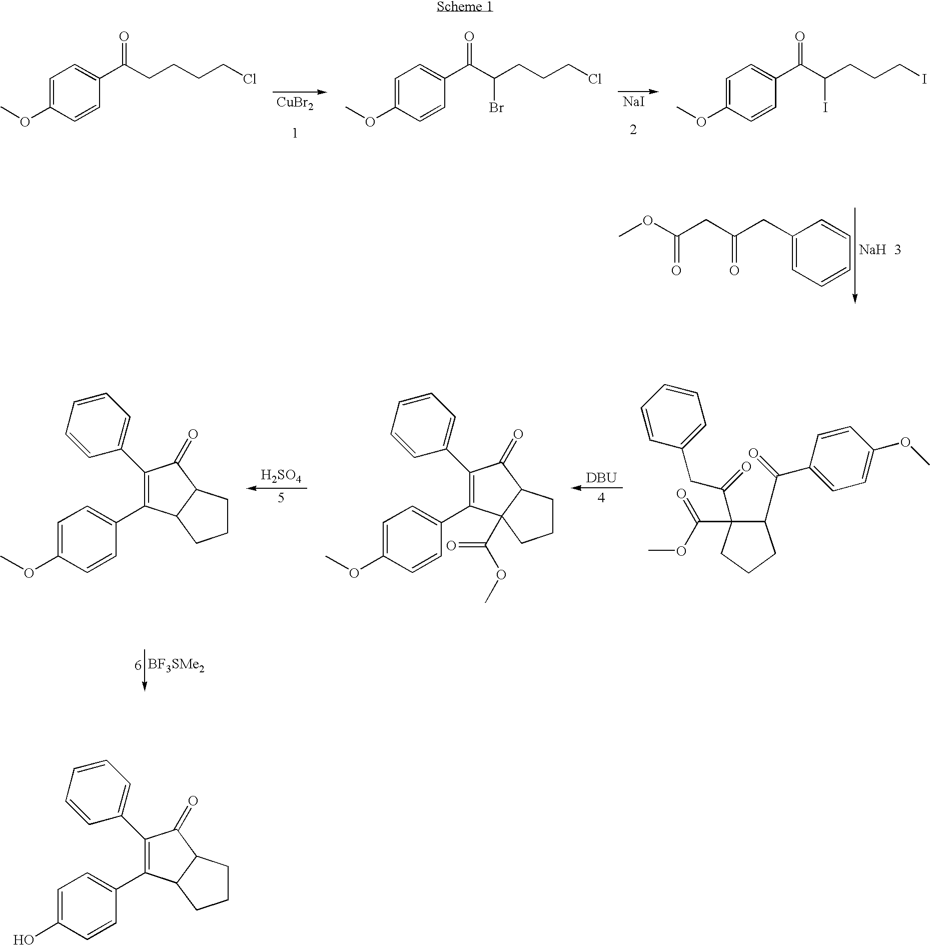

Step 1. 5-Chloro-1-(4-methoxy-phenyl)-pentan-1-one (130 mg, 0.57 mmol) was dissolved in EtOAc and CuBr₂ (170 mg, 0.76 mmol) was added. The reaction was stirred at reflux for 6 h to give 50% conversion. Another portion of 170 mg CuBr₂ was added and reflux was continued for 16 hours. The concentrated reaction was purified on silica using a Heptane/CH₂Cl₂ gradient to give 138 mg 2-Bromo-5-chloro-1-(4-methoxy-phenyl)-pentan-1-one.
Step 2. 2-Bromo-5-chloro-1-(4-methoxy-phenyl)-pentan-1-one (15 mg, 0.026 mmol) and 0.40 g, 2.7 mmol NaI was dissolved in 10 mL acetone and refluxed for 16 hours. DCM and H2O were added, layers were separated and dried using a phase separator. Concentration gave 2,5-Diiodo-1-(4-methoxy-phenyl)-pentan-1-one which was used without further purification.
Step 3. 3-Oxo-4-phenyl-butyric acid methyl ester (15 mg, 0.074 mmol) was dissolved in 1 mL THF and NaH (20 mg, 0.074 mmol) was added. The reaction was stirred for 10 min. 2,5-Diiodo-1-(4-methoxy-phenyl-pentan-1-one (30 mg, 0.068 mmol) in 1 mL of THF was added dropwise. Stirring at reflux continued for 60 hours. Workup: Et2O and HCl, dry MgSO4. Concentration gave 2-(4-Methoxy-benzoyl)-1-phenylacetyl-cyclopentanecarboxylic acid methyl ester which was used in the next step without purification.
Step 4. 2-(4-Methoxy-benzoyl)-1-phenylacetyl-cyclopentanecarboxylic acid methyl ester (0.068 mmol) was dissolved in 1 mL THF and 300 μL DBU was added. The reaction was stirred over night at room temperature. Workup: 1M HCl/Et₂O, dry MgSO₄, concentration gave 2-(4-Methoxy-benzoyl)-1-phenylacetyl-cyclopentanecarboxylic acid methyl ester which was used in the next step without purification.
Step 5. 2-(4-Methoxy-benzoyl)-1-phenylacetyl-cyclopentanecarboxylic acid methyl ester was dissolved in 3 mL AcOH and 300 μL 3M H₂SO₄ was added. The reaction was stirred at reflux for 16 hours. Workup: H₂O/Et₂O, dry MgSO₄, concentration gave a crude product which was purified by preparative HPLC to give 7 mg 3-(4-Methoxy-phenyl)-2-phenyl-4,5,6,6a-tetrahydro-3aH-pentalen-1-one.
Step 6. 3-(4-Methoxy-phenyl)-2-phenyl-4,5,6,6a-tetrahydro-3aH-pentalen-1-one (8 mg, 0.026 mmol) was dissolved in 2 mL CH₂Cl₂ and 0.1 mL of BF₃:SMe₂ was added and stirred for 60 hours at room temperature. CH₂Cl₂ and water were added and the layers were separated and dried using a phase separator. Purification using preparative HPLC gave 6.63 mg 3-(4-Hydroxy phenyl)-2-phenyl-4,5,6,6a-tetrahydro-3aH-pentalen-1-one. ES/MS m/z: 291.29 (pos. M+H), 289.29 (neg. M−H); ¹H NMR (d⁶-Acetone, 500 MHz): d 7.28 (m, 5H); 7.17 (m, 2H); 6.77 (m, 2H); 3.92 (ddd, J=2.5, 6.3, 9.1 Hz, 1H); 2.94 (ddd, J=1.9, 6.0, 8.8 Hz, 1H); 1.96 (m, 2H); 1.83 (m, 1H); 1.61 (m, 1H); 1.54 (m, 1H); 1.32 (m, 1H).

### Example 2

### 2-Bromo-3-(4-hydroxy-phenyl)-4,5,6,6a-tetrahydro-3aH-pentalen-1-one (E2)

The title compound was synthesized according to the method outlined in scheme 2.

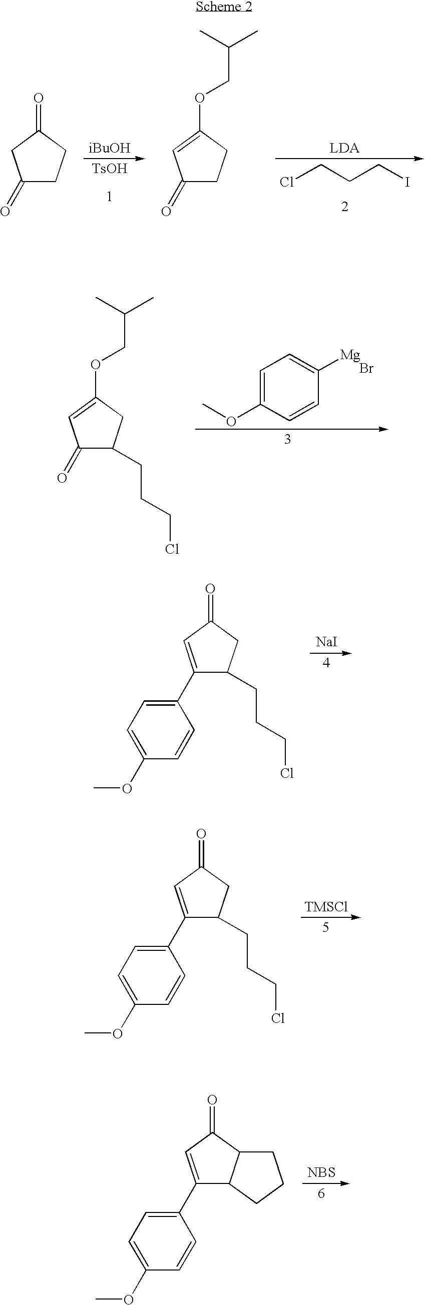

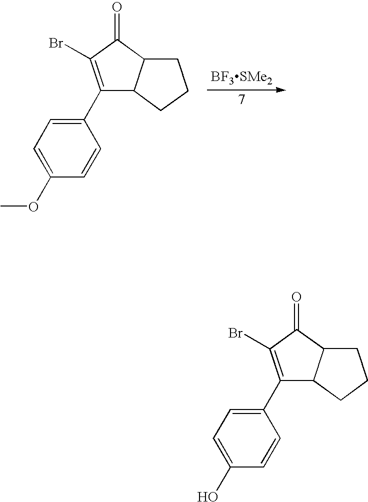

Step 1. A mixture of cyclopentanedione, p-toluensulfonic acid monohydrate (0.1 eq.) and isobutyl alcohol (6 eq.) in toluen (1 mL/mmol) was stirred for 18 hours at 85° C. The solvents were removed under vacuum and the resulting residue was diluted with ethylacetate and washed with brine. The phases were separated and the organic solvents were evaporated to give crude product which was filtrated through silica. 3-Isobutoxy-cyclopent-2-enone was obtained as yellowish oil in about 95% yield.
Step 2. To a solution of diisopropylamine (1.1 eq.) and THF (1.5 mL/mmol of 2) n-BuLi (1.1 eq.) was added drop wise at 0° C. under dry conditions. After 10 minutes the solution was cooled to −78° C. and cautiously treated with a cold solution of 3-Isobutoxy-cyclopent-2-enone and THF (0.75 mL/mmol), under N2 and keeping the inner temperature below −68° C. The yellow solution was stirred for 45 minutes at −78° C. Thereafter a cold solution of chloroiodopropane (1.5 eq.) and DMPU (0.75 ml/mmol of 3-Isobutoxy-cyclopent-2-enone) was added drop wise, under N₂ and keeping the inner temperature below −68° C. The reaction was allowed to slowly reach room temperature. Dilution with water and saturated NH₄Cl (aq) solution, extraction with diethylether, washing with water and brine, separation and drying over Na₂SO₄ followed by removal of organic solvents under reduced pressure gave the crude product as a brown residue which was purified on silica (E/H 0:1-3:7). 5-(3-Chloro-propyl)-3-isobutoxy-cyclopent-2-enone was obtained in 50% yield.
Step 3. To a solution of 5-(3-Chloro-propyl)-3-isobutoxy-cyclopent-2-enone in anhydrous THF (5 ml/mmol) at −10° C. was added 4-methoxy phenylmagnesiumbromide (2 eq.) with a syringe. The reaction was stirred for 3 hours at room temperature. It was quenched with 1M HCl, extracted with ethylacetate, washed with brine, dried over Na₂SO₄ and purified on silica (F/H 0:1-4:6). 4-(3-Chloro-propyl)-3-(4-methoxy-phenyl)-cyclopent-2-enone was obtained as pale yellow syrup.
Step 4. A mixture of the 4-(3-Chloro-propyl)-3-(4-methoxy-phenyl)-cyclopent-2-enone and sodium iodide (8 eq.) in acetone (5 mL/mmol) was refluxed over night. Dilution with DCM and washing with water gave a crude product which was filtrated through silica. 4-(3-Iodo-propyl)-3-(4-methoxy-phenyl)-cyclopent-2-enone was obtained as a pale yellow solid.
Step 5. To a solution of 4-(3-Iodo-propyl)-3-(4-methoxy-phenyl)-cyclopent-2-enone in acetonitrile (5 mL/mmol), was added trimethylsilyl chloride (1.5 eq.) and Et₃N (1.6 eq.). The yellow solution was stirred over night at room temperature. Evaporation of organic solvents and purification of the crude product on silica (E/H 0:1-3:7) gave 3-(4-Methoxy-phenyl)-4,5,6,6a-tetrahydro-3aH-pentalen-1-one as a pale yellow oil.
Step 6. To a solution of 3-(4-Methoxy-phenyl)-4,5,6,6a-tetrahydro-3aH-pentalen-1-one in anhydrous DCM (10 mL/mmol) was added NBS (1.05 eq.) at 0° C. The mixture was stirred over night at 4° C. The red solution was diluted with DCM and poured into ice-water. Separation and drying using a phase separator followed by removal of organic solvents under reduced pressure gave a red crude residue which was filtrated through silica. 2-Bromo-3-(4-methoxy-phenyl)-4,5,6,6a-tetrahydro-3aH-pentalen-1-one was obtained as pale yellow oil.
Step 7. 2-Bromo-3-(4-methoxy-phenyl)-4,5,6,6a-tetrahydro-3aH-pentalen-1-one (532 mg, 1.73 mmol) was dissolved in 60 mL CH₂Cl₂ and cooled to 0° C. 3 mL BF₃:SMe₂ was added and the ice bath was removed. After stirring 3 h at room temp the reaction was analyzed using LC/MS. An additional 4 mL of BF₃:SMe₂ was added and the reaction was stirred over night. CH₂Cl₂ and water was added, the phases were separated and dried using a phase separator. The crude product was purified using preparative HPLC. 226 mg pure 2-Bromo-3-(4-hydroxy-phenyl)-4,5,6,6a-tetrahydro-3aH-pentalen-1-one was obtained. ES/MS m/z: 293.21, 295.17 (pos. M+H), 291.18, 293.21 (neg. M−H); ¹H NMR (d⁶-Acetone, 500 MHz): d 7.94 (m, 2H), 7.00 (m, 2H), 3.99 (ddd, J=2.8, 6.9, 9.1, 1H), 2.98 (m, 1H), 1.84 (m, 3H), 1.61 (m, 1H), 1.51 (m, 1H), 1.27 (m, 1H).

### Example 3

### (3aR,6aS)-2-Bromo-3-(4-hydroxy-phenyl)-4,5,6,6a-tetrahydro-3aH-pentalen-1-one (E3)

The title compound was synthesized according to the method outlined in scheme 3.

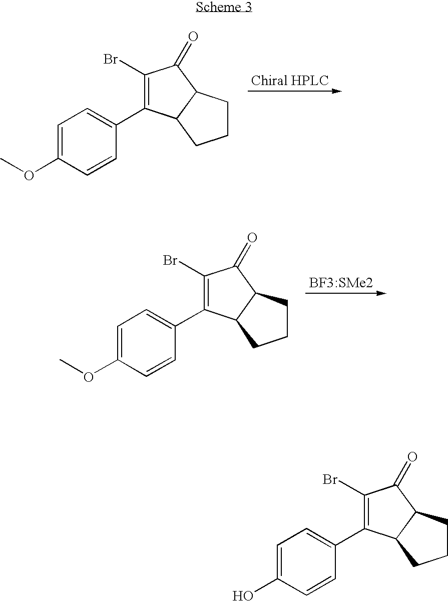

Racemic 2-Bromo-3-(4-methoxy-phenyl)-4,5,6,6a-tetrahydro-3aH-pentalen-1-one was resolved by chiral HPLC on a Reprosil Chiral-NR column using n-heptan/IPA/TFA 97/3/0.1% as mobile phase. The fractions from the first enantiomer to elute were collected and concentrated. The enantiomer was then dissolved in dichloromethane (1 ml) and cooled to 0° C. BF₃S(CH₃)₂ was added and the temperature was allowed to reach RT over night. Water and CH₂Cl₂ were added and the layers were separated. Purification using preparative HPLC gave 1.17 mg (3aR,6aS)-2-Bromo-3-(4-hydroxy-phenyl)-4,5,6,6a-tetrahydro-3aH-pentalen-1-one. ES/MS m/z: 293.14, 295.17 (pos. M+H), 291.18, 293.14 (neg. M−H).

### Example 4

### (3aS,6aR)-2-Bromo-3-(4-hydroxy-phenyl)-4,5,6,6a-tetrahydro-3aH-pentalen-1-one (E4)

Racemic 2-Bromo-3-(4-methoxy-phenyl)-4,5,6,6a-tetrahydro-3aH-pentalen-1-one was resolved by chiral HPLC on a Reprosil Chiral-NR column using n-heptan/IPA/TFA 97/3/0.1% as mobile phase. The fractions from the second enantiomer to elute were treated as in example 3 to give (3aS,6aR)-2-Bromo-3-(4-hydroxy-phenyl)-4,5,6,6a-tetrahydro-3aH-pentalen-1-one. ES/MS m/z: 293.14, 295.17 (pos. M+H), 291.18, 293.18 (neg. M−H).

### Example 5

### 2-Bromo-3-(4-hydroxy-phenyl)-5-methyl-4,5,6,6a-tetrahydro-3aH-pentalen-1-one (E5)

The title compound was synthesized using the procedures described in example 2. ES/MS m/z: 307.16, 309.13 (pos. M+H), 305.18, 307.19 (neg. M−H); ¹H NMR (d⁶-Acetone, 500 MHz): d 7.96 (m, 2H [overlapping diasteromers]); 7.01 (m, 2H[overlapping diasteromers]); 4.00 (m, 1H [overlapping diasteromers]); 3.04 (m, 1H [one diasteromers]); 3.00 (m, 1H [one diasteromers]); 2.35-1.42 (several m, 5H [overlapping diasteromers]); 0.94 (two apparent d, 3H [overlapping diasteromers])

### Example 6

### 2-Bromo-5-ethyl-3-(4-hydroxy-phenyl)-4,5,6,6a-tetrahydro-3aH-pentalen-1-one (E6)

The title compound was synthesized using the procedures described in example 2. ES/MS m/z: 323.19, 321.16 (pos. M+H), 321.18, 319.21 (neg. M−H);

### Example 7

### 2-Chloro-3-(4-hydroxy-phenyl)-4,5,6,6a-tetrahydro-3aH-pentalen-1-one (E7)

3-(4-Methoxy-phenyl)-4,5,6,6a-tetrahydro-3aH-pentalen-1-one (3.7 mg, 0.016 mmol) was dissolved in DMF (1 ml) and cooled to 0° C. N-Chlorosuccinimide (2.2 mg, 0.016 mmol) was added and the temperature was allowed to reach RT over night. EtOAc and 1M HCl were added and the phases were separated. After evaporation of the solvents, the residue was passed through a short plug of silica with Heptane/EtOAc 4:1 as eluents. The crude (3.8 mg) was dissolved in dichloromethane (1 ml) and cooled to 0° C. BF₃S(CH₃)₂ was added and the temperature was allowed to reach RT over night. The reaction was quenched with methanol and the solvents were evaporated. EtOAc and water were added and the phases were separated. After evaporation of the solvents, the residue was purified by flash chromatography with Heptane/EtOAc 3:1 as eluents to provide 1.7 mg of 2-Chloro-3-(4-hydroxy-phenyl)-4,5,6,6a-tetrahydro-3aH-pentalen-1-one. ES/MS m/z: 249.31 (pos. M+H), 247.31 (neg. M−H); ¹H NMR (MeOD, 500 MHz): d 7.84 (m, 2H); 6.82 (m, 2H); 3.85 (m, 1H); 2.89 (m, 1H); 1.64 (m, 1H); 1.55 (m, 1H); 1.45 (m, 1H); 1.18 (m, 3H).

### Example 8

### 3-(4-Hydroxy-phenyl)-1-oxo-1,3a,4,5,6,6a-hexahydro-pentalene-2-carbonitrile (E8)

2-Bromo-3-(4-methoxy-phenyl)-4,5,6,6a-tetrahydro-3aH-pentalen-1-one (6.5 mg, 0.021 mmol), copper cyanide (38 mg, 0.42 mmol) and 1-Methyl-2-pyrrolidinone (1 ml) were mixed in a microwave vial and put under nitrogen. The reaction was run at 220° C. for 1 h. DCM and water were added and the phases were separated on a phase separator. The solvents were evaporated and the residue was purified by preparative HPLC. The residue was dissolved in DCM and cooled to 0° C. under nitrogen. BF₃S(Me)₂ was added and the temperature was allowed to reach RT over night. The reaction was quenched with MeOH and the solvents were evaporated. The residue was purified by preparative HPLC to provide 1 mg of 3-(4-Hydroxy-phenyl)-1-oxo-1,3a,4,5,6,6a-hexahydro-pentalene-2-carbonitrile. ES/MS m/z: 240.23 (pos. M+H), 238.24 (neg. M−H); ¹H NMR (MeOD, 500 MHz): d 8.06 (m, 2H), 6.97 (m, 2H), 4.10 (m, 1H), 3.05 (m, 1H), 2.09 (m, 1H), 1.95-1.85 (m, 2H), 1.70 (m, 1H), 1.64 (m, 1H) and 1.31 (m, 1H).

### Example 9

### 3-(4-Hydroxy-phenyl)-2-trifluoromethyl-4,5,6,6a-tetrahydro-3aH-pentalen-1-one (E9)

2-Bromo-3-(4-methoxy-phenyl)-4,5,6,6a-tetrahydro-3aH-pentalen-1-one (27 mg, 0.088 mmol), Methyl fluorosulfonyldifluoroacetate (84 mg, 0.44 mmol), and copper iodide (22 mg, 0.11 mmol) were mixed in dry DMF under nitrogen. The reaction mixture was stirred at 80° C. for 24 h. 1M HCl and EtOAc were added, the phases were separated and the solvents were evaporated. The crude was dissolved in 5 ml DCM and cooled to 0° C. under nitrogen. BF₃S(Me)₂ (340 mg, 2.6 mmol) was added and the temperature was allowed to reach RT over night. The reaction was quenched by methanol and the solvents were evaporated. The residue was purified by preparative HPLC to provide 1.48 mg 3-(4-Hydroxy-phenyl)-2-trifluoromethyl-4,5,6,6a-tetrahydro-3aH-pentalen-1-one. ES/MS m/z: 283.28 (pos. M+H), 281.26 (neg. M−H); ¹H NMR (MeOD, 500 MHz): d 7.39 (m, 2H), 6.89 (m, 21-1), 3.91 (m, 1H), 3.00 (m, 1H), 1.92 (m, 1H), 1.86 (m, 1H), 1.77 (m, 1H), 1.61 (m, 1H), 1.43 (m, 1H) and 1.23 (m, 1H).

### Example 10

### 2-Cyclopropyl-3-(4-hydroxy-phenyl)-4,5,6,6a-tetrahydro-3aH-pentalen-1-one (E10)

2-Bromo-3-(4-methoxy-phenyl)-4,5,6,6a-tetrahydro-3aH-1-pentalen-1-one (10 mg, 0.033 mmol), Cyclopropylboronic acid (5.6 mg, 0.065 mmol), Tetrakis(triphenylphosphine)palladium(0) (3.8 mg, 0.0033 mmol) and CsCO₃ (21 mg, 0.065 mmol) were mixed in dioxane (1 ml) and water (1 ml) in a microwave vial under nitrogen. The reaction was run in a microwave reactor at 150° C. for 15 min. The solvents were evaporated and the residue was dissolved in DCM. Water was added and the phases were separated on a phase separator. The residue was passed through a short plug of silica with EtOAc as eluent and the solvents were evaporated. The crude was dissolved in DCM (1 ml) and cooled to 0° C. under nitrogen. BF₃S(Me)₂ (16 mg, 0.12 mmol) was added and the temperature was allowed to reach RT over night. The reaction was quenched by methanol and the solvents were evaporated. The residue was purified by preparative HPLC to provide 3.57 mg 2-Cyclopropyl-3-(4-hydroxy-phenyl)-4,5,6,6a-tetrahydro-3aH-pentalen-1-one. ES/MS m/z: 255.28 (pos. M+H), 253.25 (neg. M−H); ¹H NMR (CDCl₃, 500 MHz): d 7.52 (d, 2H); 6.90 (d, 2H); 163 (m, 1H); 2.82 (m, 1H); 1.95 (m, 1H); 1.78 (m, 1H); 1.60 (m, 2H); 1.52 (m, 1H); 1.27 (m, 1H); 0.94 (m, 2H), 0.87 (m, 1H), 0.70 (m, 1H).

### Example 11

### 2,2-Dimethyl-propionic acid 4-(2-bromo-3-oxo-3,3a,4,5,6,6a-hexahydro-pentalen-1-yl)-phenyl ester (E11)

The Starting Material (97 Mg, 0.33 Mmol) and Pivaloyl Chloride (400 Mg, 3.3 Mmol) were Mixed in 10 Ml of pyridine under nitrogen. The reaction was run at RT over night. Pyridine was evaporated and DCM and water were added. The phases were separated on a phase separator and evaporated. The residue was purified by flash chromatography with EtOAc/n-Heptane 3:7 as the eluent to provide 121 mg of product. ES/MS m/z: 379.27 (pos. M+H), 377.20 (neg. M−H).

### Example 12

### 2-Bromo-6a-fluoro-3-(4-hydroxy-phenyl)-4,5,6,6a-tetrahydro-3aH-pentalen-1-one (E12)

2,2,6,6-Tetramethylpiperidine (7.9 mg, 0.06 mmol) was dissolved in THF and cooled to −78° C. under nitrogen atmosphere. n-BuLi (0.06 mmol) was added and the temperature was allowed to 0° C. for 30 min. A mixture of 2,2-Dimethyl-propionic acid 4-(2-bromo-3-oxo-3,3a,4,5,6,6a-hexahydro-pentalen-1-yl)-phenyl ester (21 mg, 0.06 mmol) and DMPU (7.9 mg, 0.06 mmol) in THF was added dropwise over 10 min and the temperature was allowed to reach RT for 1 h. N-fluorobenzenesulfonimide (23 mg, 0.07 mmol) was added and the reaction mixture was stirred at RT over night. The solvents were evaporated and the crude was dissolved in methanol. A 50% solution of NaOH in EtOH was added and the mixture was stirred at RT for 30 min. 1M HCl and DCM were added and the phases were separated on a phase separator. After evaporation of the solvents, the residue was purified by prep-HPLC. ES/MS m/z: 311.1 (pos. M+H), 309.1 (neg. M−H); ¹H NMR (MeOD, 500 MHz): d 7.92 (m, 2H), 6.93 (m, 2H), 3.93 (m, 1H), 2.18 (m, 1H), 2.11-2.03 (m, 2H), 1.88 (m, 1H), 1.52 (m, 1H) and 1.43 (m, 1H).

### Example 13

### 2-Bromo-3-(4-hydroxy-phenyl)-6a-methyl-4,5,6,6a-tetrahydro-3aH-pentalen-1-one (E13)

Diisopropylamine (2.9 mg, 0.029 mmol) was dissolved in TI-IF and cooled to −78° C. under nitrogen atmosphere. n-BuLi (0,029 mmol) was added and after 10 min a mixture of 2-Bromo-3-(4-methoxy-phenyl)-4,5,6,6a-tetrahydro-3aH-pentalen-1-one (8 mg, 0.026 mmol) and DMPU (3.3 mg, 0.026 mmol) in THF was added dropwise over 10 min. After 30 min, iodomethane (18 mg, 0.18 mmol) was added and the temperature was allowed to reach RT for 2 h. Water and DCM were added and the phases were separated on a phase separator. The solvents were evaporated and the residue was dissolved in 1 ml of DCM. BF₃S(Me)₂ (0.2 ml in 2 ml of DCM) was added and the reaction mixture was stirred at RT over night. The reaction was quenched with methanol and the solvents were evaporated. The residue was purified by prep-HPLC to provide 3 mg of product. ES/MS m/z: 309.2 (pos. M+H), 307.2 (neg. M−H); ¹H NMR (MeOD, 500 MHz): d 7.93 (m, 2H), 6.91 (m, 2H), 3.53 (dd, J=9.1, 1.9 Hz, 1H), 2.03-1.90 (m, 2H), 1.65 (m, 1H), 1.54-1.48 (m, 2H), 1.29 (s, 31-1) and 1.26 (m, 1H).

### Example 14

### 3-(4-Hydroxy-phenyl)-3a,4,7,7a-tetrahydro-inden-1-one (E14)

The title compound was synthesized according to the method outlined in scheme 4.

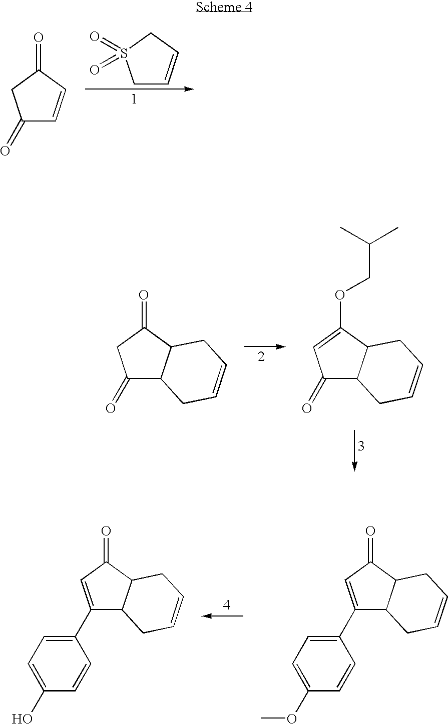

Step 1. A mixture of the cyclopent-4-ene-1,3-dione (230 mg, 2.39 mmol) and toluene (1 mL) was stirred until all starting material was in solution in a pressure safe tube. Sulfolene (848 mg, 7.18 mmol) was added and the tube was sealed and heated in an oil bath at 135° C. over night. Evaporation of the organic solvents and filtration through silica gave 180 mg 3a,4,7,7a-Tetrahydro-indene-1,3-dione.
Step 2. 3-Isobutoxy-3a,4,7,7a-tetrahydro-inden-1-one was synthesized according to step 2 in example 2. Purification on silica (EtOAc/n-Heptane 0:1-2:8) gave 85 mg of the intermediate.
Step 3. 3-(4-Methoxy-phenyl)-3a,4,7,7a-tetrahydro-inden-1-one was synthesized according to step 3 in example 2. Purification on silica (EtOAc/n-Heptane 0:1-3:7) gave 85 mg of the intermediate.
Step 4. 3-Isobutoxy-3a,4,7,7a-tetrahydro-inden-1-one was demethylated according to step 7 in example 2. Purification on silica (EtOAc/n-Heptane 0:1-4:6) gave 5 mg 3-(4-Hydroxy-phenyl)-3a,4,7,7a-tetrahydro-inden-1-one. ES/MS m/z: 227.2 (pos. M+H); H¹ NMR (d⁶-Acetone, 500 MHz): d 7.64 (d, 2H, J=8.83 Hz), 6.96 (d, 2H, J=8.83 Hz), 6.35 (s, 1H), 5.82 (m, 1H), 5.68 (m, 1H), 3.81 (m, 1H), 2.71 (m, 1H), 2.45 (m, 2H), 2.24 (m, 1H), 2.03 (m, 1H).

### Example 15

### 3-(4-Hydroxy-phenyl)-3a,4,5,6,7,7a-hexahydro-inden-1-one (E15)

The title compound was synthesized according to the method outlined in scheme 5.

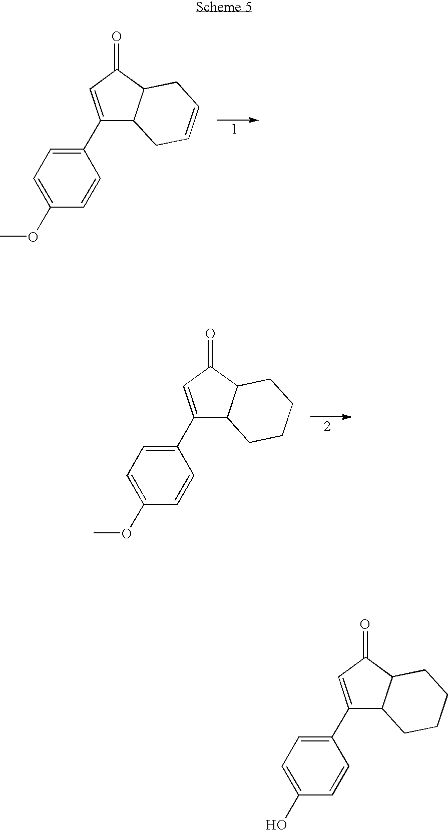

Step 1. A mixture of the 3-(4-Methoxy-phenyl)-3a,4,7,7a-tetrahydro-inden-1-one (13 mg, 0.05 mmol) and Palladium on carbon (10%) in heptane/EtOAc (0.5 mL) was hydrogenated over night at 3 psi using a hydrogen generator. The mixture was filtrated and the organic solvents were evaporated. Purification on p-HPLC(C8, 21.2×50 mm, neutral, 30-40% MeCN over 20 minutes, 50 mL/min) gave 8 mg 3-(4-Methoxy-phenyl)-3a,4,5,6,7,7a-hexahydro-inden-1-one.
Step 2. 3-(4-Methoxy-phenyl)-3a,4,5,6,7,7a-hexahydro-inden-1-one was demethylated according to step 7 in example 2. Purification on silica (EtOAc/n-Heptane 0:1-4:6) gave 5 mg 3-(4-Hydroxy-phenyl)-3a,4,5,6,7,7a-hexahydro-inden-1-one. ES/MS m/z: 229.2 (pos. M+H); H¹ NMR (CD₃CN, 500 MHz): d 7.62 (d, 2H, J=8.83 Hz), 6.89 (d, 2H, J=8.83 Hz), 6.31 (s, 1H), 3.47 (m, 1H), 2.60 (m, 1H), 2.17 (m, 1H), 2.09 (m, 1H), 1.59 (m, 3H), 1.33 (m, 1H), 1.09 (m, 1H), 0.93 (m, 1H).

### Example 16

### 2-Bromo-3-(4-hydroxy-phenyl)-3a,4,5,6,7,7a-hexahydro-inden-1-one (E16)

The title compound was synthesized from 3-(4-Methoxy-phenyl)-3a,4,5,6,7,7a-hexahydro-inden-1-one according to step 6 and 7 in example 2. ES/MS m/z: 307.1 (M+H); H¹ NMR (CDCl₃, 500 MHz): d 7.82 (d, 2H, J=8.83 Hz), 6.94 (d, 2H, J=8.83 Hz), 3.49 (m, 1H), 2.75 (m, 1H), 2.18 (m, 1H), 2.00 (m, 1H), 1.75 (m, 1H), 1.56 (m, 2H), 1.29 (m, 2H), 1.10 (m, 1H).

### Example 17

### 2-Bromo-3-(4-hydroxy-phenyl)-4,5,6,6a-tetrahydro-3aH-pentalen-1-one oxime (E17)

2-Bromo-3-(4-methoxy-phenyl)-4,5,6,6a-tetrahydro-3aH-pentalen-1-one (0.05 mmol), 60 μL pyridine, and NH₂OH—HCl (0.80 mmol) in 2 mL EtOH was stirred at 60° C. over night. The reaction was cooled, and the solvent was evaporated in vacuum. The residue was purified by preparative HPLC. Collecting the tubes containing product gave 3 mg which was deprotected using 0.1 mL BF3 in 2 mL DCM at room temperature over night. The residue was again purified by preparative HPLC. Collecting the tubes containing product gave 3.26 mg 2-Bromo-3-(4-hydroxy-phenyl)-4,5,6,6a-tetrahydro-3aH-pentalen-1-one oxime. ES/MS m/z: 308.14-310.14 (pos. M+H), 306.16-308.16 (neg. M−H); H¹ NMR (CDCl₃, 500 MHz): d 7.65 (m, 2H); 6.92 (m, 2H); 6.37 (s, 1H); 3.82 (m, 1H); 3.52 (m, 1H); 1.97 (m, 1H); 1.87 (m, 1H).); 1.77 (m, 1H); 1.56 (m, 1H); 1.42 (m, 2H).

### Example 18

### N-[4-(2-Bromo-3-oxo-3,3a,4,5,6,6a-hexahydro-pentalen-1-yl)-phenyl]-acetamide (E 18)

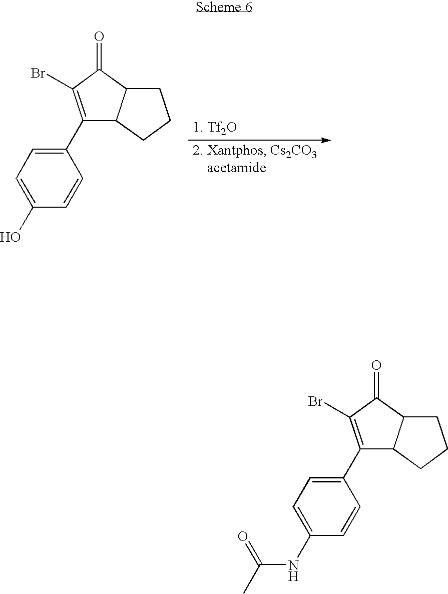

To a solution of the 2-Bromo-3-(4-hydroxy-phenyl)-4,5,6,6a-tetrahydro-3aH-pentalen-1-one (50 mg, 0.17 mmol) and lutidine (48 μL, 0.41 mmol) in CH₂Cl₂ (4 mL) at 0° C., was added triflic anhydride (69 μL, 0.41 mmol). After 30 minutes at 0° C., the ice bath was removed and the dark solution was stirred at room temperature for 1 hour. Dilution with DCM was followed by washing with water and brine. Filtration through silica gave 50 mg of triflic ester.

The triflic ester was dissolved in dry toluene (1.1 mL) and added to a reaction vial charged with Pd2(dba)3 (2 mg, 0.002 mmol), Xantphos (4 mg, 0.007 mmol), acetamide (8 mg, 0.14 mmol) and Cs2CO3 (54 mg, 0.17 mmol) under argon. The resulting mixture was stirred at 90° C. for 16 hours. Dilution with ethylacetate and filtration through a syringe filter gave crude product which was purified on P-HPLC (C8, 21.2×50 mm, acidic, 20-50% MeCN over 15 minutes, 50 mL/min). 1 mg of product was obtained as colourless oil. ES/MS m/z: 334.5 (pos. M+H); H¹ NMR (CD3CN, 500 MHz): d 7.90 (d, 2H, J=8.83 Hz), 7.71 (d, 2H, J=8.83 Hz), 3.91 (m, 1H), 2.99 (m, 1H), 2.09 (s, 31-1), 1.82 (m, 3H), 1.59 (m, 1H), 1.44 (m, 1H), 1.28 (m, 1H)

### Examples 19-56

The following compounds were prepared in like manner to the preceding examples (if not stated otherwise R⁹ and R¹⁹ are hydrogen):

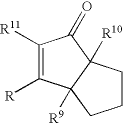

| | |
|---|---|
| E 19 | 3-(4-Hydroxy-phenyl)-4,5,6,6a-tetrahydro-3aH-pentalen-1-one |

| | |
|---|---|
| R¹¹ = H | R = 4-hydroxy-phenyl |

ES/MS m/z: 215.2 (pos. M + H); ¹H NMR (MeOD, 500 MHz): d 7.66(d, 2H, J = 8.83 Hz), 6.88(d, 2H,
J = 8.83 Hz), 6.40(s, 1H), 3.86(t, 1H, J = 7.88 Hz), 2.92(m, 1H), 1.96(m, 1H), 1.89(m, 1H), 1.78(m,
1H), 1.65(m, 2H), 1.27(m, 1H)

| | |
|---|---|
| E 20 | 2-Bromo-3-(3-bromo-4-hydroxy-phenyl)-4,5,6,6a-tetrahydro-3aH-pentalen-1-one |

| | |
|---|---|
| R¹¹ = Br | R = 3-bromo-4-hydroxy-phenyl |

ES/MS m/z: 372.8 (pos. M + H), 3.70.9 (neg. M − H).

| | |
|---|---|
| E 21 | 2-Bromo-3-(3-chloro-4-hydroxy-phenyl)-4,5,6,6a-tetrahydro-3aH-pentalen-1-one |

| | |
|---|---|
| R¹¹ = Br | R = 3-chloro-4-hydroxy-phenyl |

ES/MS m/z: 329.12 (pos. M + H), 327.2 (neg. M − H).

| | |
|---|---|
| E 22 | 2-Bromo-3-(3,5-dichloro-4-hydroxy-phenyl)-4,5,6,6a-tetrahydro-3aH-pentalen-1-one |

| | |
|---|---|
| R¹¹ = Br | R = 3,5-dichloro-4-hydroxy-phenyl |

ES/MS m/z: 363.1 (pos. M + H), 361.1 (neg. M − H).

| | |
|---|---|
| E 23 | 2-Bromo-3-(3-fluoro-4-hydroxy-phenyl)-4,5,6,6a-tetrahydro-3aH-pentalen-1-one |

| | |
|---|---|
| R¹¹ = Br | R = 3-fluoro-4-hydroxy-phenyl |

ES/MS m/z: 313.2 (pos. M + H), 311.2 (neg. M − H); ¹H NMR (MeOD, 500 MHz): d 3.31 ppm, 7.83
(dd, J = 12.6, 2.0 Hz, 1H), 7.71(dd, J = 8.8, 2.0 Hz, 1H), 7.04(t, J = 8.8 Hz, 1H), 3.95(m, 1H),
3.03(m, 1H), 1.92-1.82(m, 3H), 1.65(m, 1H), 1.53(m, 1H) and 1.27(m, 1H).

| | |
|---|---|
| E 24 | 3-(4-Hydroxy-3-methyl-phenyl)-4,5,6,6a-tetrahydro-3aH-pentalen-1-one |

| | |
|---|---|
| R¹¹ = H | R = 4-Hydroxy-3-methyl-phenyl |

ES/MS m/z: 227.2 (pos. M + H); ¹H NMR (CD3CN, 500 MHz): d 7.56(d, 1H, J = 1.89 Hz), 7.47(dd,
1H, J = 1.89, 8.20 Hz), 6.87(d, 1H, J = 8.20 Hz), 6.34(s, 1H), 3.79(m, 1H), 2.83(m, 1H), 2.21(s, 3H),
1.91(m, 1H), 1.82(m, 1H), 1.73(m, 1H), 1.60(m, 2H), 1.25(m, 1H)

| | |
|---|---|
| E 25 | 3-(2-Fluoro-4-hydroxy-phenyl)-4,5,6,6a-tetrahydro-3aH-pentalen-1-one |

| | |
|---|---|
| R¹¹ = H | R = 2-Fluoro-4-hydroxy-phenyl |

ES/MS m/z: 231.2 (pos. M + H); ¹H NMR (CD3CN, 500 MHz): d 7.57(t, 1H, J = 8.83 Hz), 6.75(dd,
1H, J = 2.52, 8.83 Hz), 6.69(dd, 1H, J = 2.52, 13.56 Hz), 6.41(s, 1H), 3.85(m, 1H), 2.79(m, 1H), 1.89
(m, 1H), 1.81(m, 1H), 1.75(m, 1H), 1.57(m, 2H), 1.24(m, 1H)

| | |
|---|---|
| E 26 | 2-Bromo-3-(4-hydroxy-3-methyl-phenyl)-4,5,6,6a-tetrahydro-3aH-pentalen-1-one |

| | |
|---|---|
| R¹¹ = Br | R = 4-hydroxy-3-methyl-phenyl |

ES/MS m/z: 307.2 (pos. M + H); ¹H NMR (CDCl3, 500 MHz): d 7.75(s, 1H), 7.71(dd, 1H, J = 2.21,
8.51 Hz), 6.89(d, 1H, J = 8.51 Hz), 3.83(m, 1H), 3.02(m, 1H), 2.31(s, 3H), 1.99(m, 1H), 1.82(m,
2H), 1.60(m, 2H), 1.30(m, 1H)

| | |
|---|---|
| E 27 | 2-Bromo-3-(2-fluoro-4-hydroxy-phenyl)-4,5,6,6a-tetrahydro-3aH-pentalen-1-one |

| | |
|---|---|
| R¹¹ = Br | R = 2-fluoro-4-hydroxy-phenyl |

ES/MS m/z: 309.1 (pos. M + H); ¹H NMR (d⁶-Acetone, 500 MHz): d 7.50(t, 1H, J = 8.83 Hz), 6.84(dd,
1H, J = 2.52, 8.52 Hz), 6.75(dd, 1H, J = 2.52, 12.61 Hz), 3.90(m, 1H), 3.02(m, 1H), 1.84(s, 2H), 1.69
(m, 1H), 1.61(m, 1H), 1.48(m, 1H), 1.28(m, 1H)

| | |
|---|---|
| E 28 | 3-(3-Fluoro-4-hydroxy-phenyl)-4,5,6,6a-tetrahydro-3aH-pentalen-1-one |

| | |
|---|---|
| R¹¹ = H | R = 3-Fluoro-4-hydroxy-phenyl |

ES/MS m/z: 309.1 (pos. M + H); ¹H NMR (d⁶-Acetone, 500 MHz): d 7.51(dd, 1H, J = 2.21, 12.30 Hz),
7.44(dd, 1H, J = 2.21, 8.83 Hz), 7.05(t, 1H, J = 8.83 Hz), 6.37(s, 1H), 3.84(m, 1H), 2.87(m, 1H),
1.84(m, 1H), 1.63(m, 2H), 1.25(m, 1H), 0.88(m, 2H).

| | |
|---|---|
| E 29 | 2-Bromo-3-(3-chloro-5-fluoro-4-hydroxy-phenyl)-4,5,6,6a-tetrahydro-3aH-pentalen-1- |
| | one |

| | |
|---|---|
| R¹¹ = Br | R = 3-chloro-5-fluoro-4-hydroxy-phenyl |

ES/MS m/z: 345.5-347.5 (pos. M + H), 343.5-345.5 (neg. M − H); ¹H NMR (CDCl3, 500 MHz): d 7.75
(m, 1H); 7.68(dd, J = 2.2, 11.3 Hz, 1H); 5.93(br s, 1H, OH); 3.75(ddd, J = 2.2, 6.0, 8.5 Hz, 1H); 3.05
(ddd, J = 2.2, 6.0, 9.1 Hz, 1H); 2.00(m, 1H);1.85(m, 2H). ); 1.66(m, 1H); 1.55(m, 1H); 1.32(m, 1H).

| | |
|---|---|
| E 30 | 2-Chloro-3-(3-chloro-5-fluoro-4-hydroxy-phenyl)-4,5,6,6a-tetrahydro-3aH-pentalen-1- |
| | one |

| | |
|---|---|
| R¹¹ = Cl | R = 3-chloro-5-fluoro-4-hydroxy-phenyl |

ES/MS m/z: 301.8-303.5 (pos. M + H), 299.5-301.5 (neg. M − H);

| | |
|---|---|
| E 31 | 3-(4-Hydroxy-phenyl)-2-thiophen-2-yl-4,5,6,6a-tetrahydro-3aH-pentalen-1-one |

| | |
|---|---|
| R¹¹ = thiophen-2-yl | R = 4-Hydroxy-phenyl |

ES/MS m/z: 297.2 (pos. M + H), 295.2 (neg. M − H); ¹H NMR (CDCl3, 500 MHz): d 7.31(d, 2H);
7.28(d, 1H); 7.21(d, 1H); 7.0(m, 1H); 6.90(d, 2H); 3.71(m, 1H); 3.05(m, 1H); 2.12(m, 1H); 1.90(m,
1H); 1.74(m, 1H), 1.35(m, 3H)

| | |
|---|---|
| E 32 | 3-(4-Hydroxy-phenyl)-2-(3-methyl-thiophen-2-yl)-4,5,6,6a-tetrahydro-3aH-pentalen-1- |
| | one |

| | |
|---|---|
| R¹¹ = 3-methyl-thiophen-2-yl | R = 4-Hydroxy-phenyl |

ES/MS m/z: 311.3 (pos. M + H), 309.3 (neg. M − H); ¹H NMR (CDCl3, 500 MHz): d 7.30(m, 2H); 6.86
(d, 1H); 6.79(d, 1H); 6.76(d, 2H); 3.84(m, 1H); 3.04(m, 1H); 2.07(m, 2H); 1.84(s, 3H); 1.64(m,
2H); 1.36(m, 2H).

| | |
|---|---|
| E 33 | 3-(4-Hydroxy-phenyl)-2-prop-1-ynyl-4,5,6,6a-tetrahydro-3aH-pentalen-1-one |

| | |
|---|---|
| R¹¹ = prop-1-ynyl | R = 4-Hydoxy-phenyl |

ES/MS m/z: 253.2 (pos. M + H), 251.3 (neg. M − H).

| | |
|---|---|
| E 34 | 2-Ethynyl-3-(4-hydroxy-phenyl)-4,5,6,6a-tetrahydro-3aH-pentalen-1-one |

| | |
|---|---|
| R¹¹ = Ethynyl | R = 4-Hydroxy-phenyl |

ES/MS m/z: 239.2 (pos. M + H), 237.2 (neg. M − H); ¹H NMR (MeOD, 500 MHz): d 8.17(m, 2H),
6.89(m, 2H), 3.98(s, 1H), 3.95(m, 1H), 2.95(m, 1H), 2.00(m, 1H), 1.91(m, 1H), 1.83(m, 1H), 1.67-1.58
(m, 2H) and 1.24(m, 1H).

| | |
|---|---|
| E 35 | 2-[3-(4-Hydroxy-phenyl)-1-oxo-1,3a,4,5,6,6a-hexahydro-pentalen-2-yl]-thiophene-3- |
| | carbonitrile |

| | |
|---|---|
| R¹¹ = 3-Cyano-thiophen-2-yl | R = 4-Hydroxy-phenyl |

ES/MS m/z: 322.2 (pos. M + H), 320.2 (neg. M − H); ¹H NMR(MeOD, 500 MHz): d 7.68(d, J = 5.2
Hz, 1H), 7.34(d, J = 5.2 Hz, 1H), 7.28(m, 2H), 6.76(m, 2H), 4.05(m, 1H), 3.10(m, 1H), 2.03-1.87(m,
3H), 1.71-1.61(m, 2H) and 1.39(m, 1H).

| | |
|---|---|
| E 36 | 2-Furan-2-yl-3-(4-hydroxy-phenyl)-4,5,6,6a-tetrahydro-3aH-pentalen-1-one |

| | |
|---|---|
| R¹¹ = furan-2-yl | R = 4-Hydroxy-phenyl |

ES/MS m/z: 281.6 (pos. M + H), 279.6 (neg. M − H); ¹H NMR(d⁶-Acetone, 500 MHz): d 7.42(dd,
J = 1.6, 1.0 Hz; 1H), 7.44(m, 2H), 6.91-6.88(m, 3H), 6.49(dd, J = 3.2, 1.6 Hz, 1H), 3.87(m, 1H),
2.95(m, 1H), 1.93(m, 1H), 1.85-1.74(m, 2H), 1.57(m, 1H), 1.51(m, 1H) and 1.23(m, 1H).

| | |
|---|---|
| E 37 | 3-(4-Hydroxy-phenyl)-2-vinyl-4,5,6,6a-tetrahydro-3aH-pentalen-1-one |

| | |
|---|---|
| R¹¹ = vinyl | R = 4-Hydroxy-phenyl |

ES/MS m/z: 241.5 (pos. M + H), 239.5 (neg. M − H); ¹H NMR(d⁶-Acetone, 500 MHz): d 7.44(m, 2H),
6.98(m, 2H), 6.46(dd, J = 17.7, 11.4 Hz, 1H), 6.35(dd, J = 17.7, 2.8 Hz, 1H), 5.31(dd, J = 11.4, 2.8
Hz), 3.78(m, 1H), 2.84(m, 1H), 1.88(m, 1H), 1.80-1.68(m, 2H), 1.54(m, 1H), 1.44(m, 1H) and 1.16(m,
1H).

| | |
|---|---|
| E 38 | 3-(4-Hydroxy-phenyl)-2-(2-methoxy-thiazol-4-yl)-4,5,6,6a-tetrahydro-3aH-pentalen-1- |
| | one |

| | |
|---|---|
| R¹¹ = 2-methoxy-thiazol-4-yl | R = 4-Hydroxy-phenyl |

ES/MS m/z: 328.6 (pos. M + H), 326.6 (neg. M − H); ¹H NMR(d⁶-Acetone, 500 MHz): d 7.40(m, 2H),
7.28(s, 1H), 6.86(m, 2H), 3.87(m, 1H), 3.80(s, 3H), 2.93(m, 1H), 1.94(m, 1H), 1.85-1.76(m, 2H),
1.58(m, 1H), 1.53(m, 1H) and 1.25(m, 1H).

| | |
|---|---|
| E 39 | 3-(4-Hydroxy-phenyl)-2-thiazol-4-yl-4,5,6,6a-tetrahydro-3aH-pentalen-1-one |

| | |
|---|---|
| R¹¹ = thiazol-4-yl | R = 4-Hydroxy-phenyl |

ES/MS m/z: 298.6 (pos. M + H), 326.6 (neg. M − H); ¹H NMR(d⁶-Acetone, 500 MHz): d 8.91(d,
J = 1.9 Hz, 1H), 7.79(d, J = 1.9 Hz, 1H, 7.32(m, 2H), 6.81(m, 2H), 3.94(m, 1H), 2.98(m, 1H),
1.96(m, 1H), 1.89-1.79(m, 2H), 1.60(m, 1H), 1.55(m, 1H) and 1.28(m, 1H).

| | |
|---|---|
| E 40 | 3-(4-Hydroxy-phenyl)-2-thiazol-2-yl-4,5,6,6a-tetrahydro-3aH-pentalen-1-one |

| | |
|---|---|
| R¹¹ = thiazol-2-yl | R = 4-Hydroxy-phenyl |

ES/MS m/z: 298.6 (pos. M + H), 326.6 (neg. m − H); ¹H NMR(d⁶-Acetone, 500 MHz): d 8.89(s, 1H),
8.00(s, 1H), 7.37(m, 2H), 6.95(m, 2H), 3.86(m, 1H), 3.01(m, 1H), 1.96(m, 1H), 1.85(m, 1H), 1.74
(m, 1H), 1.61(m, 1H), 1.55(m, 1H) and 1.29(m, 1H).

| | |
|---|---|
| E 41 | 3-(4-Hydroxy-phenyl)-2-(2-methyl-allyl)-4,5,6,6a-tetrahydro-3aH-pentalen-1-one |

| | |
|---|---|
| R¹¹ = 2-methyl-allyl | R = 4-Hydroxy-phenyl |

ES/MS m/z: 267.6 (pos. M + H), 269.6 (neg. M − H); ¹H NMR(d⁶-Acetone, 500 MHz): d 7.50(m, 2H),
6.94(m, 2H), 4.75(m, 1H), 4.48(m, 1H), 3.85(m, 1H), 3.10(d, J = 16.4 Hz, 1H), 2.83-2.79(m, 2H),
1.85-1.72(m, 6H), 1.58(m, 1H), 1.49(m, 1H) and 1.22(m, 1H).

| | |
|---|---|
| E 42 | 3-(4-Hydroxy-phenyl)-2-((E)-propenyl)-4,5,6,6a-tetrahydro-3aH-pentalen-1-one |

| | |
|---|---|
| R¹¹ = (E)-propenyl | R = 4-Hydroxy-phenyl |

ES/MS m/z: 253.5 (pos. M + H), 255.6 (neg. M − H); ¹H NMR(d⁶-Acetone, 500 MHz): d 7.43(m, 2H),
6.97(m, 2H), 6.91(m, 1H), 6.16(m, 1H), 3.73(m, 1H), 2.81(m, 1H), 1.86(m, 1H), 1.77(m, 3H), 1.75-
1.65(m, 2H), 1.52(m, 1H), 1.42(m, 1H) and 1.15(m, 1H).

| | |
|---|---|
| E 43 | 3-(4-Hydroxy-phenyl)-2-((Z)-propenyl)-4,5,6,6a-tetrahydro-3aH-pentalen-1-one |

| | |
|---|---|
| R¹¹ = (Z)-propenyl | R = 4-Hydroxy-phenyl |

ES/MS m/z: 253.5 (pos. M + H), 255.6 (neg. M − H); ¹H NMR(d⁶-Acetone, 500 MHz): d 7.66(m, 2H),
6.92(m, 2H), 5.95(m, 1H), 5.77(m, 1H), 3.85(m, 1H), 2.81(m, 1H), 1.90-1.81(m, 2H), 1.76(m, 1H),
1.61-1.51(m, 2H), 1.42(dd, J = 6.6, 1.6 Hz, 3H) and 1.23(m, 1H).

| | |
|---|---|
| E 44 | 3-(4-Hydroxy-phenyl)-2-(3-methyl-but-2-enyl)-4,5,6,6a-tetrahydro-3aH-pentalen-1-one |

| | |
|---|---|
| R¹¹ = 3-methyl-but-2-enyl | R = 4-Hydroxy-phenyl |

ES/MS m/z: 281.7 (pos. M + H), 283.6 (neg. M − H); ¹H NMR(d⁶-Acetone, 500 MHz): d 7.46(m, 2H),
6.95(m, 2H), 5.02(m, 1H), 3.76(m, 1H), 3.06(dd, J = 14.5, 5.4 Hz, 1H), 2.96(dd, J = 14.5, 7.3 Hz, 1H),
2.76(m, 1H), 1.72(m, 1H), 1.64(s, 6H), 1.52(m, 1H), 1.43(m, 1H), 1.39-1.26(m, 2H) and 1.16(m,
1H).

| | |
|---|---|
| E 45 | 2-Acetyl-3-(4-hydroxy-phenyl)-4,5,6,6a-tetrahydro-3aH-pentalen-1-one |

| | |
|---|---|
| R¹¹ = Acetyl | R = 4-Hydroxy-phenyl |

ES/MS m/z: 255.6 (pos. M + H), 257.5 (neg. M − H); ¹H NMR(d⁶-Acetone, 500 MHz): d 7.50(m, 2H),
6.93(m, 2H), 3.94(m, 1H), 2.92(m, 1H), 2.30(s, 3H), 1.91-1.75(m, 3H), 1.60(m, 1H), 1.52(m, 1H)
and 1.27(m, 1H).

| | |
|---|---|
| E 46 | 3-(4-Hydroxy-phenyl)-2-thiophen-3-yl-4,5,6,6a-tetrahydro-3aH-pentalen-1-one |

| | |
|---|---|
| R¹¹ = thiophen-3-yl | R = 4-Hydroxy-phenyl |

ES/MS m/z: 297.6 (pos. M + H), 2.95.6 (neg. M − H); ¹H NMR(d⁶-Acetone, 500 MHz): d 7.61(dd,
J = 2.8, 1.3 Hz, 1H), 7.34(dd, J = 5.0, 2.8 Hz, 1H), 7.32(m, 2H), 6.86(m, 2H), 6.85(dd, J = 5.0, 1.3 Hz,
1H), 3.84(m, 1H), 2.93(m, 1H), 1.96(m, 1H), 1.82(m, 1H), 1.76(m, 1H), 1.58(m, 1H), 1.52(m, 1H) and
1.26(m, 1H)

| | |
|---|---|
| E 47 | 3-(4-Hydroxy-phenyl)-2-isopropenyl-4,5,6,6a-tetrahydro-3aH-pentalen-1-one |

| | |
|---|---|
| R¹¹ = isopropenyl | R = 4-Hydroxy-phenyl |

ES/MS m/z: 255.6 (pos. M + H), 253.5 (neg. M − H); ¹H NMR(d⁶-Acetone, 500 MHz): d 7.58(m ,2H),
6.91(m, 2H), 5.15(m, 1H), 4.85(m, 1H), 3.79(m, 1H), 2.80(m, 1H), 1.87(m, 1H), 1.82-1.71(m, 5H),
1.55(m, 1H), 1.50(m, 1H) and 1.20(m, 1H).

| | |
|---|---|
| E 48 | 3-(4-Hydroxy-phenyl)-2-(1-methyl-1H-pyrrol-2-yl)-4,5,6,6a-tetrahydro-3aH-pentalen-1- |
| | one |

| | |
|---|---|
| R¹¹ = 1-methyl-1H-pyrrol-2-yl | R = 4-Hydroxy-phenyl |

ES/MS m/z: 294.7 (pos. M + H), 292.6 (neg. M − H); ¹H NMR(d⁶-Acetone, 500 MHz): d 7.25(m, 2H),
6.80(m, 2H), 6.73(dd, J = 2.5, 1.6 Hz, 1H), 6.05(dd, J = 3.5, 2.5 Hz, 1H), 5.87(dd, J = 3.5, 1.6 Hz,
1H), 3.94(m, 1H), 2.92(m, 1H), 1.98-1.90(m, 2H), 1.80(m, 1H), 1.66-1.59(m, 2H) and 1.30(m, 1H).

| | |
|---|---|
| E 49 | Benzoic acid 4-(2-bromo-3-oxo-3,3a,4,5,6,6a-hexahydro-pentalen-1-yl)-phenyl ester |

| | |
|---|---|
| R¹¹ = Br | R = 4-Benzoic acid-phenyl ester |

ES/MS m/z: 397.16 (pos. M + H), 395.14 (neg. M − H); ¹H NMR(MeOD, 500 MHz): d 8.21(m, 2H),
8.02(m, 2H), 7.72(m, 1H), 7.58(m, 2H), 7.44(m, 2H), 4.03(m, 1H), 3.09(m, 1H), 1.93-1.89(m, 2H),
1.85(m, 1H), 1.67(m, 1H), 1.54(m, 1H) and 1.32(m, 1H).

| | |
|---|---|
| E 50 | 2-Bromo-3-(4-dimethylamino-phenyl)-4,5,6,6a-tetrahydro-3aH-pentalen-1-one |

| | |
|---|---|
| R¹¹ = Br | R = 4-Dimethylamino-phenyl |

ES/MS m/z: 320.02 (pos. M + H); ¹H NMR(CDCl₃, 500 MHz): d 8.01(m, 2H), 6.75(m, 2H), 3.84(m,
1H), 3.07(s, 6H), 3.00(m, 1H), 2.00(m, 1H), 1.83(m, 2H), 1.62(m, 2H) and 1.32(m, 1H).

| | |
|---|---|
| E 51 | 2-Bromo-3-(4-hydroxy-2,5-dimethyl-phenyl)-4,5,6,6a-tetrahydro-3aH-pentalen-1-one |

| | |
|---|---|
| R¹¹ = Br | R = 4-Hydroxy-2,5-dimethyl-phenyl |

ES/MS m/z: 321.01 (pos. M + H), 319.1 (neg. M − H); ¹H NMR(d6-Acetone, 500 MHz): d 6.98(s, 1H),
6.80(s, 1H), 3.73(m, 1H), 3.01(m, 1H), 2.20(s, 3H), 2.18(s, 3H), 1.90(m, 1H), 1.82(m, 1H), 1.68-
1.51(m, 3H) and 1.33(m, 1H).

| | |
|---|---|
| E 52 | 3-(6-Hydroxy-naphthalen-2-yl)-4,5,6,6a-tetrahydro-3aH-pentalen-1-one |

| | |
|---|---|
| R¹¹ = H | R = 6-Hydroxy-naphtalen-2-yl |

ES/MS m/z: 265.08 (pos. M + H), 263.10 (neg. M − H); ¹H NMR(d6-Acetone, 500 MHz): d 8.96(bs,
1H), 8.26(d, J = 1.10 Hz, 1H), 7.93(d J = 8.83 Hz, 1H), 7.82(dd J = 8.83, 1.89 Hz 1H), 7.77(d J = 8.83
Hz, 1H), 7.26(d J = 2.52 Hz, 1H), 7.22(dd J = 8.83, 2.52 Hz, 1H), 6.58(d J = 1.0 Hz, 1H), 4.01(m,
1H), 2.90(m, 1H), 2.04(m, 1H), 1.89(m, 1H), 1.81-1.67(m, 2H), 1.64(m, 1H) and 1.28(m, 1H).

| | |
|---|---|
| E 53 | 2-Bromo-3-(4-hydroxy-3,5-dimethyl-phenyl)-4,5,6,6a-tetrahydro-3aH-pentalen-1-one |

| | |
|---|---|
| R¹¹ = Br | R = 4-Hydroxy-3,5-dimethyl-phenyl |

ES/MS m/z: 321.01 (pos. M + H), 319.09 (neg. M − H); ¹H NMR(CDCl₃, 500 MHz): d 7.59(s, 2H),
4.99(s, 1H), 3.82(m, 1H), 3.02(m, 1H), 2.32(s, 6H), 2.00(m, 1H), 1.88-1.74(m, 2H), 1.66-1.53(m,
2H) and 1.31(m, 1H).

| | |
|---|---|
| E 54 | 2-Bromo-3-(4-hydroxy-2-methyl-phenyl)-4,5,6,6a-tetrahydro-3aH-pentalen-1-one |

| | |
|---|---|
| R¹¹ = Br | R = 4-Hydroxy-2-methyl-phenyl |

ES/MS m/z: 307.02 (pos. M + H), 305.05 (neg. M − H); ¹H NMR(CDCl₃, 500 MHz): d 7.00(d J = 8.20
Hz, 1H), 6.83-6.77(m, 2H), 5.80(bs, 1H), 3.61(m, 1H), 3.07(m, 1H), 2.24(s, 3H), 2.05(m, 1H), 1.84
(m, 1H), 1.67(m, 1H), 1.61-1.54(m, 2H) and 1.35(m, 1H).

| | |
|---|---|
| E 55 | 3a-Bromo-3-(3,5-difluoro-4-hydroxy-phenyl)-4,5,6,6a-tetrahydro-3aH-pentalen-1-one |

| | |
|---|---|
| R¹¹ = H | R = 3,5-difluoro-4-hydroyx-phenyl |
| R⁹ = Br | |

ES/MS m/z: 328.97 (pos. M + H), 327.01 (neg. M − H); ¹H NMR(d6-Acetone, 500 MHz): d 7.70(m,
2H), 6.72(s, 1H), 3.56(dd J = 10.09, 4.41 Hz, 1H), 2.67-2.54(m, 2H), 2.17(m, 1H), 1.94(m, 1H), 1.73
(m, 1H) and 1.56(m, 1H).

| | |
|---|---|
| E 56 | 2-(3,5-Dimethyl-isoxazol-4-yl)-3-(4-hydroyx-phenyl)-4,5,6,6a-tetrahydro-3aH-pentalen-1- |
| | one |

| | |
|---|---|
| R¹¹ = 3,5-Dimethyl-isoxazol-4-yl | R = 4-Hydroxy-phenyl |

ES/MS m/z: 310.7 (pos. M + H), 308.5 (neg. M − H); ¹H NMR(d6-Acetone, 500 MHz): d 7.35(bs, 2H),
6.81(d, J = 8.83 Hz, 2H), 3.92(bs, 1H), 2.94(m, 1H), 2.82(m, 1H), 2.23(m, 1H), 1.85(m, 5H), 1.60
(s, 3H) and 1.31(m, 2H).

### Example 57

### 3-(4-Amino-3-methyl-phenyl)-2-bromo-4,5,6,6a-tetrahydro-3aH-pentalen-1-one (E57)

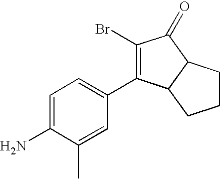

To an oven dried reaction vial loaded with 2-Bromo-3-(4-diallylamino-3-methyl-phenyl)-4,5,6,6a-tetrahydro-3aH-pentalen-1-one (0.98 g, 2.54 mmol), palladium tetrakistriphenylphosphine (58 mg, 0.05 mmol) and DCM (12 mL) was added 1,3-dimethylbarbituric acid (2.97 g, 19 mmol). After 1.5 hours stirring at room temperature the starting material was gone. The organic solvent was evaporated, followed by addition of ether. The resulting mixture was washed with sat. NaHCO₃ (aq) solution (3×), water and brine. Crude product was purified on silica (E/H 0:1-3:7). 525 mg of the title compound was obtained as a pale yellow solid.

ES/MS m/z: 306.05 (pos. M+H); ¹H NMR (CDCl₃, 500 MHz): d 7.77 (d, J=2.21 Hz, 1H), 7.73 (dd, J=8.51, 2.21 Hz, 1H), 6.80 (d, J=8.51 Hz, 1H), 3.81 (m, 1H), 2.99 (m, 1H), 2.25 (s, 3H), 1.98 (m, 1H), 1.81 (m, 2H), 1.60 (m, 2H) and 1.30 (m, 1H).

### Examples 58-60

The following compounds were prepared in like manner to the preceding example:

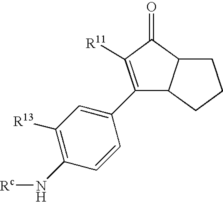

| | |
|---|---|
| E 58 | 3-(4-Amino-phenyl)-4,5,6,6a-tetrahydro-3aH-pentalen-1-one |

| | |
|---|---|
| R¹¹ = H | R¹³ = H |
| R^{c} = H | |

ES/MS m/z: 214.0 (pos. M + H); ¹H NMR (CDCl₃, 500 MHz): d 7.51(d, J = 8.51 Hz, 2H), 6.72(d, J =
8.51 Hz, 2H), 6.34(s, 1H), 3.72(m, 1H), 2.91(m, 1H), 1.98(m, 1H), 1.88(m, 1H), 1.75(m, 1H), 1.70
(m, 1H), 1.62(m, 1H) and 1.31(m, 1H)

| | |
|---|---|
| E 59 | 3-(4-Amino-phenyl)-2-bromo-4,5,6,6a-tetrahydro-3aH-pentalen-1-one |

| | |
|---|---|
| R¹¹ = Br | R¹³ = H |
| R^{c} = H | |

ES/MS m/z: 293.0 (pos. M + H); ¹H NMR(CD₃OD, 500 MHz): d 7.93(d, J = 8.83 Hz, 2H), 6.73(d, J =
8.83 Hz, 2H), 3.94(m, 1H), 2.98(m, 1H), 1.95-1.79(m, 3H), 1.63(m, 1H), 1.57(m, 1H), and 1.26(m,
1H).

| | |
|---|---|
| E 60 | 3-(4-Amino-3-bromo-phenyl)-2-bromo-4,5,6,6a-tetrahydro-3aH-pentalen-1-one |

| | |
|---|---|
| R¹¹ = Br | R¹³ = Br |
| R^{c} = H | |

ES/MS m/z: 372.0 (pos. M + H); ¹H NMR(CD₃CN, 500 MHz): d 8.13(d, 2.21 Hz, 1H), 7.81(dd,
J = 8.51, 2.21 Hz, 1H), 6.90(d, J = 8.51 Hz, 1H), 5.09(bs, 2H), 3.86(m, 1H), 2.94(m, 1H), 1.88-1.78(m,
3H), 1.59(m, 1H), 1.47(m, 1H), and 1.26(m, 1H).

### Example 61

### 2-Bromo-3-(1H-indazol-5-yl)-4,5,6,6a-tetrahydro-3aH-pentalen-1-one (E61)

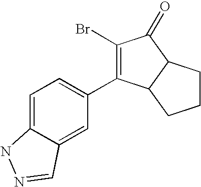

To a stirred solution of 3-(4-Amino-3-methyl-phenyl)-2-bromo-4,5,6,6a-tetrahydro-3aH-pentalen-1-one (05 g, 1.63 mmol) and DCM (13 mL) was added nitrosium tetrafluoroborate (190 mg, 1.63 mmol) at −40° C. The formed yellow mixture was stirred for 1 hour and the temperature was allowed to reach 4° C. The brown yellow mixture was diluted with DCM (13 mL) and cooled down to −40° C. again. Potassium acetate (336 mg, 3.43 mmol) and dibenzo-18-crown-6 (24 mg, 0.07 mmol) were added to the reaction solution. The ice bath was removed after 10 minutes and the red solution was allowed to reach room temperature. After 1.5 hours no starting material was left. Dilution with DCM, washing with water, extraction with DCM, separation and drying with a phase separator gave crude product. Purification on silica (E/H 0:1-1:1) gave 400 mg of the title compound as a yellow solid.

ES/MS m/z: 279.09 (pos. M+H); ¹H NMR (CDCl₃, 500 MHz): d 8.38 (s, 1H), 8.25 (s, 1H), 7.95 (dd, J=8.83, 2.21 Hz, 1H), 7.66 (d, J=8.83 Hz, 1H), 3.94 (m, 1H), 3.10 (m, 1H), 2.03 (m, 1H), 1.87 (m, 1H), 1.81 (m, 1H), 1.65 (m, 1H), 1.56 (m, 1H) and 1.35 (m, 1H).

### Example 62

### 3-(1H-Indazol-5-yl)-1-oxo-1,3a,4,5,6,6a-hexahydro-pentalene-2-carbonitrile (E62)

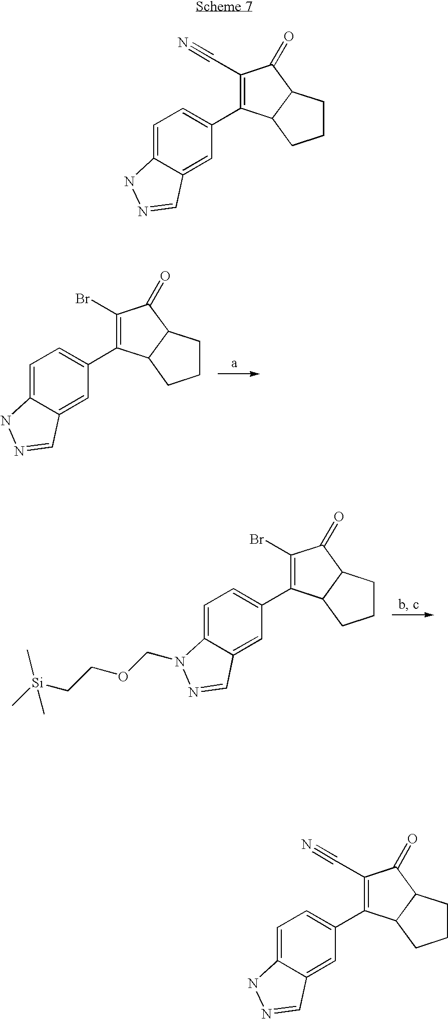

a.) The indazole (190 mg, 0.6 mmol), (2-chloromethoxy-ethyl)-trimethyl-silane (300 mg, 1.8 mmol), tetrabutyl ammonium bromide (19 mg, 0.06 mmol) and potassium hydroxide (168 mg, 3 mmol) were mixed in DCM/H₂O (1:1, 6 mL) at 0° C. The temperature was allowed to reach room temperature and the solution was stirred for 5 days. Addition of brine and extraction with DCM gave crude product which was purified on silica (1-1/E0:1-1:1). 268 mg of an orange oil was obtained.
b.) The SEM protected indazole (90 mg, 0.2 mmol) and copper cyanide (360 mg, 4 mmol) were mixed in NMP (3 mL) and heated to 180° C. After 1 hour the resulting dark mixture was diluted with EtOAc and washed with water (×4) and brine. After evaporation the obtained crude product was purified on silica (E/H 0:1-3:7) to obtain 13 mg (13% yield) of a beige residue.
c.) The SEM protected indazole (13 mg, 0.03 mmol) was mixed with HCl (3M, 1.1 mL) and EtOH (1 mL) in a micro oven safe vial. The reaction was run at 120° C. for 10 min. The mixture was neutralized by sat. NaHCO₃ and the EtOH were evaporated. DCM and water were added and the phases were separated and dried with a phase separator. Purification on silica (E/H 0:1-1:1) gave 4 mg (46% yield) of a pale yellow solid.

ES/MS m/z: 264.08 (pos. M+H); ¹H NMR (CD₃CN, 500 MHz): d 8.53 (s, 1H), 8.23 (s, 1H), 8.11 (dd, J=8.83, 1.89 Hz, 1H), 7.66 (d, J=8.83 Hz, 1H), 7.74 (d, J=8.83 Hz, 1H), 4.17 (m, 1H), 3.06 (m, 1H), 2.05 (m, 1H), 1.89 (m, 2H), 1.65 (m, 1H), 1.59 (m, 1H) and 1.35 (m, 1H).

### Examples 63-64

The following compounds were prepared in like manner to the preceding examples:

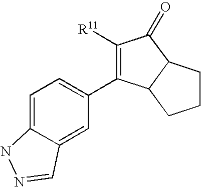

| | |
|---|---|
| E 63 | 3-(1H-Indazol-5-yl)-4,5,6,6a-tetrahydro-3aH-pentalen-1-one |

R¹¹ = H
ES/MZ m/z: 239.08 (pos. M + H); ¹H NMR (CDCl₃, 500 MHz): d 8.22(s, 1H), 8.09(s, 1H), 7.73(dd, J =
8.83, 1.26 Hz, 1H), 7.61(d, J = 8.83 Hz, 1H), 6.54(s, 1H), 3.88(m, 1H), 3.01(m, 1H), 2.02(m, 1H),
1.94(m, 1H), 1.80(m, 1H), 1.73(m, 1H), 1.66(m, 1H) and 1.33(m, 1H)

| | |
|---|---|
| E 64 | 2-[3-(1H-Indazol-5-yl)-1-oxo-1,3a,4,5,6,6a-hexahydro-pentalen-2-yl]-thiophene-3- |
| | carbonitrile |

R¹¹ = 3-carbonitrile-thiophene
ES/MS m/z: 346.11 (pos. M + H); ¹H NMR (CD₃CN, 500 MHz): d 8.07(d, J = 0.95 Hz, 1H), 7.94(dd, J =
1.58, 0.95 Hz, 1H), 7.62(d, J = 5.36 Hz, 1H), 7.53(m, 1H), 7.32(m, 2H), 4.12(m, 1H), 3.14(m, 1H),
1.98-1.88(m, 3H), 1.67(m, 1H), 1.56(m, 1H) and 1.41(m, 1H).

### Example 65

### 2-Bromo-3-(4-isobutylamino-phenyl)-4,5,6,6a-tetrahydro-3aH-pentalen-1-one (E 65)

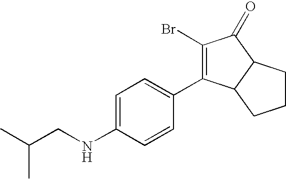

Sodium cyanoborohydride (5 mg, 0.08 mmol) was added to a solution of 3-(4-Amino-phenyl)-2-bromo-4,5,6,6a-tetrahydro-3aH-pentalen-1-one (19 mg, 0.07 mmol) and the 2-methyl-propionaldehyde (6 mg, 0.08 mmol) in methanol (1.5 mL). The mixture was stirred at room temperature overnight. The mixture was heated to 45° C. and stirred over night. Addition of THF (0.75 mL) and stirred over night at 45° C. Addition of 2 equivalents of aldehyde and NaCNBH₃. The solution was stirred for 3 days at room temperature. More aldehyde and NaCNBH₃ were added (about 12 eq.) and the mixture was stirred over night at 45° C.

The reaction mixture was poured into water and extracted with dichloromethane. Separation and drying was done by a phase separator. The organic solvent was removed under vacuum and the residue was purified by p-HPLC (neutral, 40-70% MeCN over 20 minutes, sunfire C8 short column, 25 mL/min) to provide 11 mg of the title compound.

ES/MS m/z: 348.13 (pos. M+H); ¹H NMR (CDCl₃, 500 MHz): d 7.93 (d, J=8.83 Hz, 2H), 6.69 (d, J=8.83 Hz, 2H), 3.81 (m, 1H), 3.02 (d, J=6.62 Hz, 2H), 2.99 (m, 1H), 1.99 (m, 1H), 1.94 (m, 1H), 1.82 (m, 2H), 1.61 (m, 2H), 1.31 (m, 1H) and 1.01 (d, J=6.62 Hz, 6H).

### Examples 66-68

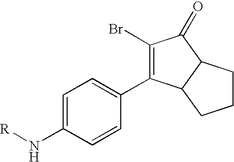

The following compounds were prepared in like manner to the preceding example:

| | |
|---|---|
| E 66 | 2-Bromo-3-(4-methylamino-phenyl)-4,5,6,6a-tetrahydro-3aH-pentalen-1-one |
| R = CH₃ | |

ES/MS m/z: 306.05 (pos. M + H); ¹H NMR (CDCl₃, 500 MHz): d 7.95 (d, J = 7.88 Hz, 2H), 6.80 (d, J = 7.88 Hz,
2H), 3.82 (m, 1H), 3.01 (m, 1H), 2.95 (s, 3H), 1.99 (m, 1H), 1.82 (m, 2H), 1.61 (m, 2H) and
1.32 (m, 1H)

| | |
|---|---|
| E 67 | 2-Bromo-3-{4-[(furan-2-ylmethyl)-amino]-phenyl}-4,5,6,6a-tetrahydro-3aH-pentalen-1- |
| | one |
| R = furan-2-ylmethyl | |

ES/MS m/z: 372.08 (pos. M + H); ¹H NMR (CDCl₃, 500 MHz): d 7.94 (d, J = 8.83 Hz, 2H), 7.38 (s,
1H), 6.76 (d, J = 8.83 Hz, 2H), 6.34 (m, 1H), 6.29 (d, J = 3.15 Hz, 1H), 4.41 (s, 1H), 3.81 (m, 1H),
3.00 (m, 1H), 2.00 (m, 1H), 1.82 (m, 2H), 1.61 (m, 2H) and 1.31 (m, 1H).

| | |
|---|---|
| E 68 | 2-Bromo-3-(4-pentylamino-phenyl)-4,5,6,6a-tetrahydro-3aH-pentalen-1-one |
| R = n-pentyl | |

ES/MS m/z: 362.12 (pos. M + H); ¹H NMR (CDCl₃, 500 MHz): d 7.95 (d, J = 8.51 Hz, 2H), 6.70 (d, J = 8.51 Hz,
2H), 3.82 (m, 1H), 3.19 (m, 2H), 2.99 (m, 1H), 1.99 (m, 1H), 1.82 (m, 2H), 1.64 (m, 4H),
1.35 (m, 5H) and 0.92 (m, 3H).

### Example 69 and 70

### 2-Bromo-3-(4-hydroxy-phenyl)-5-methylene-4,5,6,6a-tetrahydro-3aH-pentalen-1-one (E69)

### 3-(4-Hydroxy-phenyl)-5-methylene-4,5,6,6a-tetrahydro-3aH-pentalen-1-one (E70)

The title compounds were synthesized according to the method outlined in scheme 8.

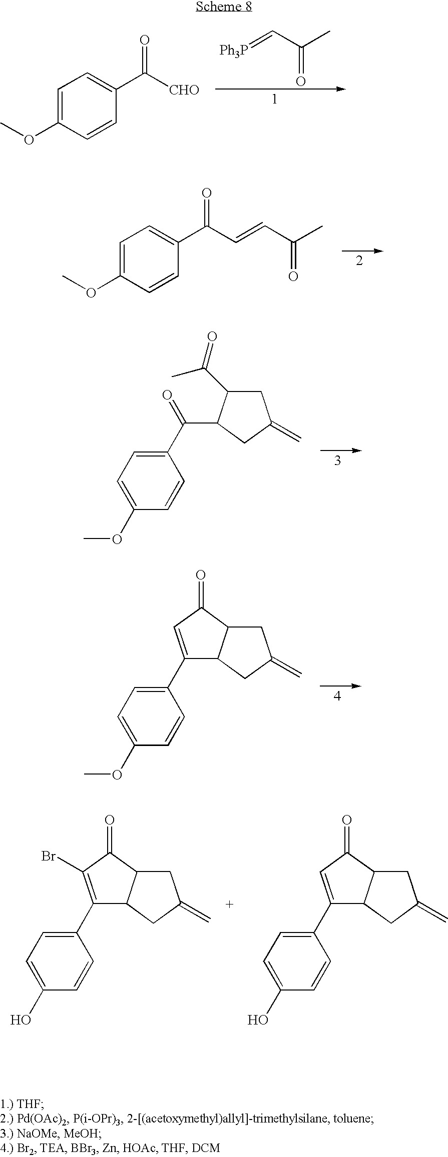

Step 1. 4-Methoxyphenylglyoxal hydrate (1.0 equiv) and triphenylphosphoranylidene-2-propanone (1.0 equiv) in THF was stirred at rt overnight. Purification was performed on silica gel with 20-30% EtOAc in n-heptane to give (E)-1-(4-methoxy-phenyl)-pent-2-ene-1,4-dione in 93% yield. ES/MS m/z: 205.02 (pos. M+H); ¹HNMR (CDCl₃, 500 MHz): d 7.96-8.03 (m, 2H), 7.70 (d, J=16.0 Hz, 1H), 7.07 (d, J=16.0 Hz, 1H), 6.95-7.01 (m, 21-1), 3.90 (s, 31-1), 2.43 (s, 3H);
Step 2. (E)-1-(4-methoxy-phenyl)-pent-2-ene-1,4-dione (1.0 equiv), Pd(OAc)₂ (0.1 eq), triiopropyl phosphate (0.8 eq) and 2-[(acetoxymethyl)allyl]-trimethylsilane (1.2 eq) in toluene were stirred at 100° C. for 22 h. The solution was concentrated and purified on silica gel with EtOAc/n-heptane (1:3). The title compound was obtained in 47% yield. ES/MS m/z: 259.14 (pos. M+H); (CDCl₃, 500 MHz): d 7.90-8.04 (m, 2H), 6.88-7.01 (m, 2H), 4.82-4.96 (m, 2H), 4.03-4.18 (m, 1H), 3.88 (s, 3H), 3.57-3.71 (m, 1H), 2.76-2.91 (m, 2H), 2.37-2.58 (m, 2H), 2.19 (s, 3H);
Step 3. 1-[2-(4-methoxy-benzoyl)-4-methylene-cyclopentyl]ethanone (1.0 equiv) and 0.5M NaOMe in MeOH (1.0 equiv) in THF was stirred at 50° C. for 1 hour and 15 min. The mixture was concentrated and dissolved in 20 mL DCM, washed with 10 mL water, and extracted with DCM (10 mL×2). The combined organic layers were concentrated and the crude product was purified on silica gel using EtOAc/n-heptane as eluents to give 3-(4-methoxy-phenyl)-5-methylene-4,5,6,6a-tetrahydro-3aH-pentalen-1-one in 69% yield. ES/MS m/z: 241.07 (pos. M+H); ¹HNMR (CDCl₃, 500 MHz): d 7.55-7.65 (m, 2H), 6.92-7.03 (m, 2H), 6.39 (d, J=1.0 Hz, 1H), 4.80-4.84 (m, 1H), 4.69-4.73 (m, 1H), 3.87 (s, 3H), 3.80-3.86 (m, 1H), 2.97-3.04 (m, 1H), 2.75-2.84 (m, 1H), 2.60-2.69 (m, 1H), 2.51-2.58 (m, 1H), 2.17-2.23 (m, 1H);
Step 4. To a solution of 3-(4-methoxy-phenyl)-5-methylene-4,5,6,6a-tetrahydro-3aH-pentalen-1-one (1.0 equiv) in THF was added Br₂ (2.5 equiv). After 5 min TEA (5 equiv) was added and the mixture was stirred at rt for 2 hrs. The reaction was filtered and washed with DCM. The solvent was concentrated and the residue was dissolved in 5 mL DCM and 1.0 M BBr₃ (10 equiv) in DCM was added at −78° C. and stirred at 4° C. overnight. Then 5 mL water was added and extracted with another 5 mL×2 DCM. The DCM layer was dried with MgSO₄, concentrated and Zn (1.04 equiv), 5 mL THF and HOAc (1.26 equiv) were added and stirred overnight. The reaction mixture was filtered, concentrated and purified with EtOAc/Heptane (1:1) to give 2-Bromo-3-(4-hydroxy-phenyl)-5-methylene-4,5,6,6a-tetrahydro-3aH-pentalen-1-one (E69) in 16% yield ES/MS m/z: 305.02 (pos. M+H), 303.03 (neg. M−H); ¹HNMR (CDCl₃/d6-Acetone, 500 MHz): d 9.11 (br s, 1H), 7.89-7.98 (m, 2H), 6.96-7.08 (m, 2H), 4.77-4.81 (m, 1H), 4.68-4.72 (m, 1H), 4.05-4.12 (m, 1H), 3.06-3.13 (m, 1H), 2.65-2.82 (m, 2H), 2.39-2.45 (m, 1H) and 2.03-2.10 (m, 1H) in 16% yield and 3-(4-Hydroxy-phenyl)-5-methylene-4,5,6,6a-tetrahydro-3aH-pentalen-1-one (E70) in 15% yield. ES/MS m/z: 227.09 (pos. M+H), 225.07 (neg. M−H); (d6-Acetone, 500 MHz): d 9.02 (br s, 1H), 7.65-7.71 (m, 2H), 6.94-6.98 (m, 2H), 6.38 (d, J=1.1 Hz, 1H), 4.75-4.78 (m, 1H), 4.67-4.70 (m, 1H), 2.80-2.95 (m, 3H), 2.57-2.65 (m, 1H), 2.39-2.45 (m, 1H) and 2.11-2.18 (m, 1H).

### Example 71

### 2-Benzyl-6-(4-hydroxy-phenyl)-2,3,3a,6a-tetrahydro-1,1-cyclopentalclpyrrol-4-one (E71)

The title compound was synthesized according to the method outlined in scheme 9.

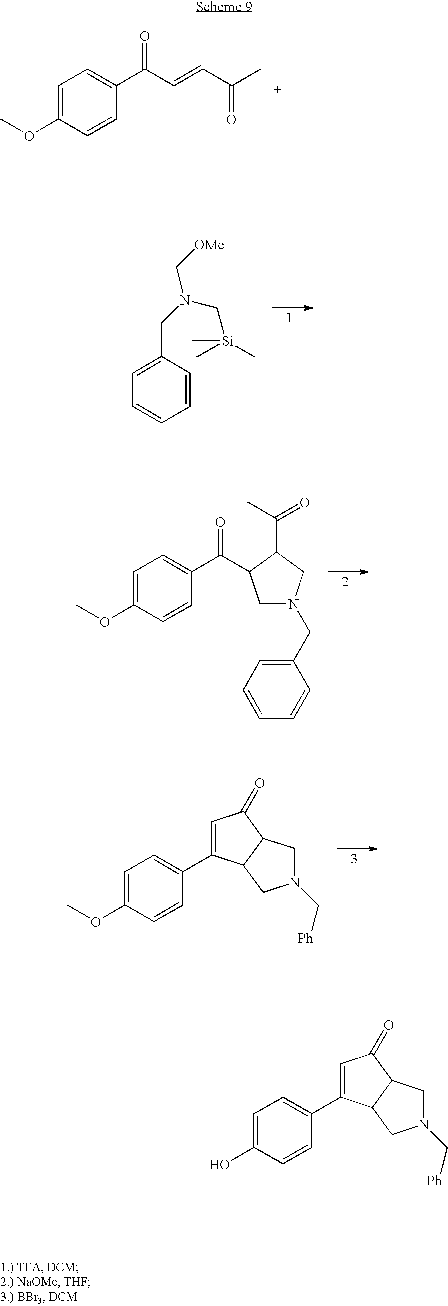

Step 1. 0.1 eq of TFA was added to a solution of (E)-1-(4-methoxy-phenyl)-pent-2-ene-1,4-dione (1.0 eq) and benzyl-methoxymethyl-trimethylsilanylmethyl-amine (1.2 eq) in DCM at 0° C. The mixture was stirred at 0° C. for 2.5 h and then at rt for another half hour. The solution was concentrated and purified on silica gel with n-Heptane/EtOAc (1:1) to give 1-[1-benzyl-4-(4-methoxy-benzoyl)-pyrrolidin-3-yl]-ethanone in 81% yield. ES/MS m/z: 338.20 (pos. M+H); ¹HNMR (CDCl₃, 500 MHz): d 7.92-7.97 (m, 2H), 7.21-7.33 (m, 5H), 6.90-6.95 (m, 2H), 4.35-4.42 (m, 1H), 3.86 (s, 3H), 3.74-3.80 (m, 1H), 3.57-3.68 (m, 2H), 3.08-3.13 (m, 1H), 2.91-2.96 (m, 1H), 2.87 (dd, J=9.3, 5.8 Hz, 1H), 2.69 (dd, J=9.3, 6.5 Hz, 1H), 2.15 (s, 3H);
Step 2. 6.25 mL 0.5 M NaOMe (1.0 eq) in MeOH was added to a solution of 1-[1-benzyl-4-(4-methoxy-benzoyl)-pyrrolidin-3-yl]-ethanone (1.0 eq) in 60 mL THF at 50° C. The reaction turned yellow and was stirred at 50° C. for 1 hour. The mixture was concentrated and dissolved in 30 mL DCM. Then 20 mL water was added and the solution was neutralized with 2 N HCl and thereafter extracted with DCM 20 mL×2. The organic layers were combined and concentrated The residue was purified with 2% MeOH in DCM and EtOAc/n-Heptane(1:1) to give 2-benzyl-6-(4-methoxy-phenyl)-2,3,3a,6a-tetrahydro-1H-cyclopenta[c]pyrrol-4-one in 74% yield. ES/MS m/z: 320.13 (pos. M+H); 7.58-7.63 (m, 2H), 7.22-7.33 (m, 5H), 6.95-7.00 (m, 2H), 6.49 (s, 1H) 3.55-4.00 (m 5H), 3.01-3.19 (m, 21-1), 2.59-2.89 (m, 2H);
Step 3. 1.0 M BBr₃ (15 equiv) in DCM was added to a solution of 2-benzyl-6-(4-methoxy-phenyl)-2,3,3a,6a-tetrahydro-1H-cyclopenta[c]pyrrol-4-one (1.0 equiv) in 5 ml, DCM and stirred overnight at rt. The reaction was quenched with 5 mL water and Na₂CO₃ (sat, aq) was added to adjust the pH to 8-9. The aqueous solution was extracted with DCM 10 mL×4, concentrated and the residue was purified on silica gel using 2% MeOH in EtOAc to give 2-Benzyl-6-(4-hydroxy-phenyl)-2,3,3a,6a-tetrahydro-1H-cyclopenta[c]pyrrol-4-one in 12% yield. ES/MS m/z: 306.1 (pos. M+H), 304.14 (neg. M−H); ¹HNMR (d6-Acetone, 500 MHz): d 7.88 (br s, 1H), 7.65-7.72 (m, 2H), 7.28-7.42 (m, 5H), 6.96-7.03 (m, 2H), 6.54 (d, J=0.82 Hz, 1H), 3.98-4.07 (m, 1H), 3.62-3.80 (m, 2H), 3.00-3.19 (m, 2H), 2.84-2.92 (m, 1H) and 2.54-2.83 (m, 2H).

### Example 72

### (rae)-(3aS,5R,6aR)-5-Bromo-3-(4-hydroxy-phenyl)-5-methyl-4,5,6,6a-tetrahydro-3aH-pentalen-1-one (E72)

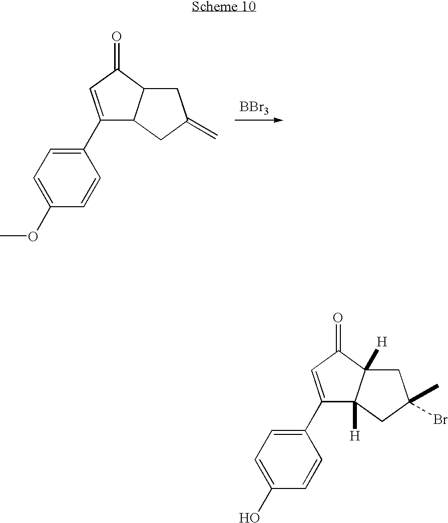

3-(4-Methoxy-phenyl)-5-methylene-4,5,6,6a-tetrahydro-3aH-pentalen-1-one (1.0 eq) was dissolved in DCM under nitrogen and cooled to 0° C. 1.0 M BBr3 (4 eq) in DCM was added dropwise and the mixture was stirred at 4° C. overnight. The reaction was quenched with water and extracted with DCM 5 mL×3, concentrated and dissolved in DMSO/DCM, and purified using 25-50% EtOAc in n-Heptane on silica gel to give (rac)-(3aS,5S,6aR)-5-Bromo-3-(4-hydroxy-phenyl)-5-methyl-4,5,6,6a-tetrahydro-3aH-pentalen-1-one and (rac)-(3aS,5R,6aR)-5-Bromo-3-(4-hydroxy-phenyl)-5-methyl-4,5,6,6a-tetrahydro-3aH-pentalen-1-one in 16% yield. ES/MS m/z: 307.03 (pos. M+H), 305.05 (neg. M−H); ¹HNMR (CDCl₃/CD₃OD, 500 MHz): d 7.44-7.50 (m, 2H), 6.81-6.88 (m, 2H), 6.32 (d, J=1.2 Hz, 1H), 3.84-3.91 (m, 1H), 3.04-3.10 (m, 1H), 2.52-2.59 (m, 1H), 2.22-2.39 (m, 3H) and 1.79 (s, 3H).

### Example 73 and 74

### (rae)-(3aS,5R,6aR)-2,5-Dibromo-3-(4-hydroxy-phenyl)-5-methyl-4,5,6,6a-tetrahydro-3aH-pentalen-1-one (E73)

### (rac)-(3aS,5S,6aR)-2,5-Dibromo-3-(4-hydroxy-phenyl)-5-methyl-4,5,6,6a-tetrahydro-3aH-pentalen-1-one (E74)

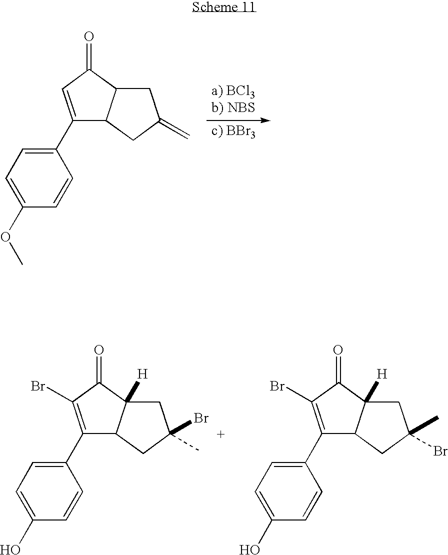

1.0M BCl₃ (20 eq) in DCM was added to a solution of 3-(4-methoxy-phenyl)-5-methylene-4,5,6,6a-tetrahydro-3aH-pentalen-1-one (1.0 equiv) in 5 mL DCM and the mixture was stirred for 48 h at rt. The reaction was quenched with water and extracted with DCM 5 mL×3. The combine organic layers were dried over MgSO₄ and concentrated. NBS (2.0 eq) and 5 mL DCM were added to the crude product and the mixture was stirred at it overnight. Then 1.0 M BBr₃ (10 eq) in DCM was added at −40° C. and the mixture was stirred at 4° C. overnight. The reaction was quenched with water and extracted with DCM 6 mL×3. The combined DCM layers were dried over MgSO₄, concentrated and the crude product was purified on silica gel using 25%-50% EtOAc in n-heptane to give (rac)-(3aS,5S,6aR)-2,5-Dibromo-3-(4-hydroxy-phenyl)-5-methyl-4,5,6,6a-tetrahydro-3aH-pentalen-1-one (E73) in 22% yield ES/MS m/z: 386.99 (pos. M+H), 384.97 (neg. M−H); ¹HNMR (CDCl₃/CD₃OD, 500 MHz): d 7.74-7.80 (m, 2H), 6.84-6.91 (m, 2H), 3.95-4.02 (m, 1H), 3.12-3.20 (m, 1H), 2.60-2.69 (m, 1H), 2.15-2.32 (m, 3H) and 1.77 (s, 3H) and (rac)-(3aS,5R,6aR)-2,5-Dibromo-3-(4-hydroxy-phenyl)-5-methyl-4,5,6,6a-tetrahydro-3aH-pentalen-1-one (E74) in 10% yield ES/MS m/z: 386.99 (pos. M+H), 384.97 (neg. M−H); ¹HNMR (CDCl₃/CD₃OD, 500 MHz): d 7.85-7.93 (m, 2H), 6.85-6.93 (m, 2H), 4.21-4.31 (m, 1H), 3.45-3.55 (m, 1H), 2.64-2.76 (m, 2H), 1.81 (s, 3H), 1.72 (dd, J=14.5, 9.3 Hz, 1H) and 1.46 (dd, J=14.7, 9.3 Hz, 1H).

### Example 75

### (rac)-(3aS,5S,6aR)-5-Chloro-3-(4-hydroxy-phenyl)-5-methyl-4,5,6,6a-tetrahydro-3aH-pentalen-1-one (E75)

1.0 M BCl₃ (20 eq) in DCM was added to a solution of 3-(4-methoxy-phenyl)-5-methylene-4,5,6,6a-tetrahydro-3aH-pentalen-1-one(1.0 eq) in 5 mL DCM and the mixture was stirred for 48 h at it. The reaction was quenched with water and the aqueous mixture was extracted with DCM 5 mL×3. The combined organic layers were dried over MgSO₄, concentrated and the residue was dissolved in 5 mL dry DCM. BF₃SMe₂ (20 eq) was added to the solution under N₂ and the mixture was stirred at it overnight. The reaction was quenched with 6 mL water and extracted with 10 mL×3 DCM. The organic layers were combined and concentrated. The crude product was purified on silica gel using 20%-50% EtOAc in n-heptane to give (rac)-(3aS,5S,6aR)-5-Chloro-3-(4-hydroxy-phenyl)-5-methyl-4,5,6,6a-tetrahydro-3aH-pentalen-1-one in 20% yield. ES/MS m/z: 263.07 (pos. M+H), 261.06 (neg. M−H); ¹HNMR (CDCl₃, 500 MHz): d 7.54-7.60 (m, 2H), 6.90-6.97 (m, 2H), 6.27 (d, J=1.1 Hz, 1H), 5.65 (s, 1H, OH), 4.11-4.20 (m, 1H), 3.40-3.49 (m, 1H), 2.55-2.72 (m, 2H), 1.81 (dd, J=14.2, 9.0 Hz, 1H), 1.69 (s, 3H) and 1.57 (dd, J=13.9, 9.2 Hz, 1H).

### Example 76-79

### (rac)-(3aS,5S,6aR)-2-Bromo-5-chloro-3-(4-hydroxy-phenyl)-5-methyl-4,5,6,6a-tetrahydro-3aH-pentalen-1-one (E76)

### (rac)-(3aS,5R,6aR)-2-Bromo-5-chloro-3-(4-hydroxy-phenyl)-5-methyl-4,5,6,6a-tetrahydro-3aH-pentalen-1-one (E77)

### (rac)-(5R,6aS)-2,3a-Dibromo-5-chloro-3-(4-hydroxy-phenyl)-5-methyl-4,5,6,6a-tetrahydro-3aH-pentalen-1-one (E78)

### (rac)-(5S,6aS)-3a-Bromo-5-chloro-3-(4-hydroxy-phenyl)-5-methyl-4,5,6,6a-tetrahydro-3aH-pentalen-1-one (E79)

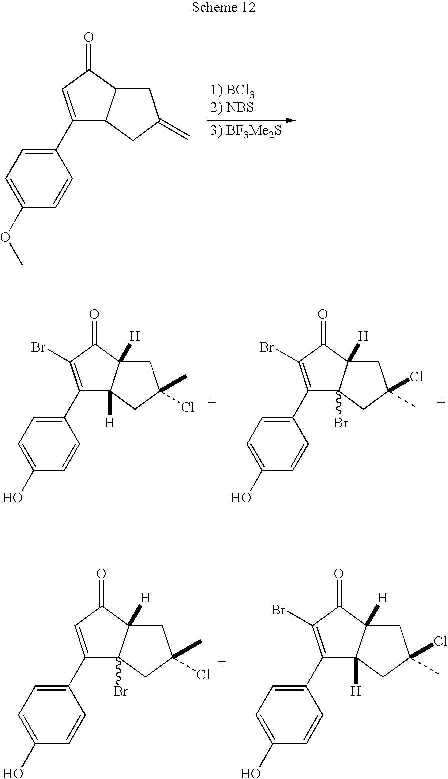

To 3-(4-methoxy-phenyl)-5-methylene-4,5,6,6a-tetrahydro-3aH-pentalen-1-one in 5 ml DCM was added 1.0 M BCl₃ (20 eq) in DCM and the mixture was stirred for 48 h. The reaction was quenched with 5 mL water and the aqueous mixture was extracted with DCM 10 mL×3. The combined organic layers were dried over MgSO₄ and concentrated to give the crude chlorinated products. 2 eq NBS were added followed by 5 mL dry DCM. The mixture was stirred at rt overnight and then concentrated. The residue was dissolved in 3 mL dry DCM and 20 eq BF₃SMe₂ were added to the solution at 0° C. under N₂. The reaction mixture was stirred at rt for 4 h, then slowly quenched with 6 mL water and the aqueous mixture was extracted with DCM 4×10 mL. The combined organic layers were concentrated and purified on silica gel using 20%-50% EtOAc in n-heptane to give (rac)-(3aS,5R,6aR)-2-Bromo-5-chloro-3-(4-hydroxy-phenyl)-5-methyl-4,5,6,6a-tetrahydro-3aH-pentalen-1-one in 5% yield. ES/MS m/z: 342.99 (pos. M+H), 341.05 (neg. M−H); ¹HNMR (CDCl₃, 500 MHz): d 7.89-7.97 (m, 2H), 6.92-7.00 (m, 2H), 5.31 (s, 1H, OH), 4.19-4.28 (m, 1H), 3.47-3.56 (m, 1H), 2.54-2.69 (m, 2H), 1.81 (dd, J=14.2, 9.2 Hz, 1H), 1.67 (s, 3H) and 1.51 (dd, J=14.0, 9.2 Hz, 1H). Further purification using 2% MeOH in DCM gave 13.7 mg, 24% yield (rac)-(3aS,5S,6aR)-2-Bromo-5-chloro-3-(4-hydroxy-phenyl)-5-methyl-4,5,6,6a-tetrahydro-3aH-pentalen-1-one ES/MS m/z: 342.98 (pos. M+H), 341.02 (neg. M−H); ¹HNMR (CDCl₃/CD₃OD, 500 MHz): d 7.73-7.78 (m, 2H), 6.85-6.89 (m, 2H), 3.90-3.97 (m, 1H), 3.10-3.17 (m, 1H), 2.49-2.55 (m, 1H), 2.07-2.24 (m, 3H) and 1.57 (s, 3H) and (rac)-(5R,6aS)-2,3a-Dibromo-5-chloro-3-(4-hydroxy-phenyl)-5-methyl-4,5,6,6a-tetrahydro-3aH-pentalen-1-one in 7% yield ES/MS m/z: 420.91 (pos. M+H), 418.91 (neg. M−H); ¹HNMR (CDCl₃, 500 MHz): d 7.72-7.75 (m, 2H), 6.95-6.98 (m, 2H), 6.27 (d, J=1.1 Hz, 1H), 5.23 (s, 1H, OH), 3.64 (dd, J=9.7, 2.7 Hz, 1H), 3.11 (dd, J=15.4, 1.9 Hz, 1H), 2.56-2.72 (m, 3H) and 1.74 (s, 3H). Further purification using 1% MeOH in DCM gave (rac)-(5S,6aS)-3a-Bromo-5-chloro-3-(4-hydroxy-phenyl)-5-methyl-4,5,6,6a-tetrahydro-3aH-pentalen-1-one in 16% yield ES/MS m/z: 343.01 (pos. M+H), 341.01 (neg. M−H); ¹HNMR (CDCl₃, 500 MHz): d 7.81-7.85 (m, 2H), 6.95-6.99 (m, 2H), 6.57 (s, 1H), 5.11 (br s, 1H, OH), 3.59 (dd, J=7.9, 5.4 Hz, 1H), 3.26 (d, J=15.2 Hz, 1H), 2.94 (d, J=15.2 Hz, 1H), 2.53-2.56 (m, 2H) and 1.78 (s, 3H).

### Example 80

### 2-Bromo-3-(2,3-difluoro-4-hydroxy-phenyl)-4,5,6,6a-tetrahydro-3aH-pentalen-1-one (E80)

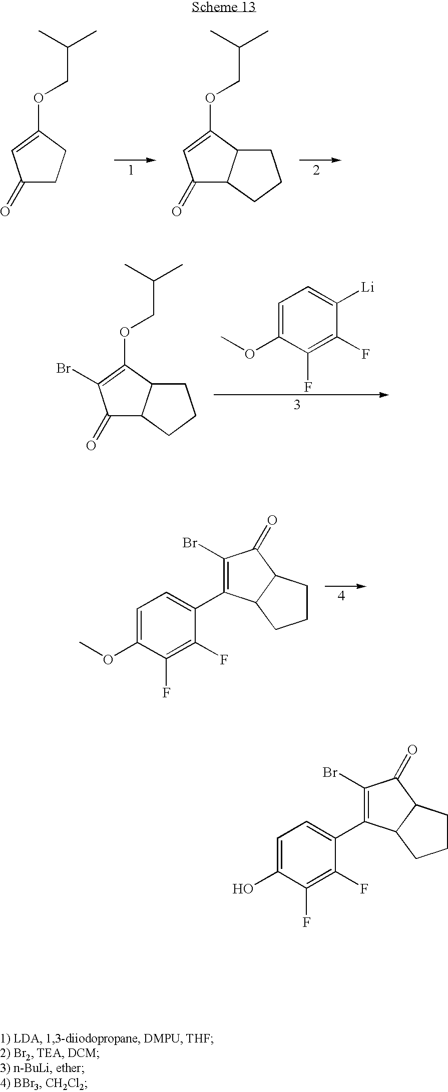

Step 1) Diisopropylamine (58 ml, 0.41 mol, 2.2 eq.) was dissolved in dry THF (1.3 l) and the solution was cooled in an ice-bath. n-BuLi (164 ml, 2.5 M in hexanes, 0.41 mol, 2.2 eq.) was added dropwise from a dropping funnel over 20 minutes. The mixture was stirred for additional 10 min. and was then cooled to −78° C. A solution of 3-Isobutoxy-cyclopent-2-enone (28.76 g, 0.186 mol, 1.0 eq.) in dry THF (180 ml) was added from a dropping funnel over 40 minutes keeping the internal reaction temperature in the range of −73 to −65° C. The resulting mixture was stirred for 25 min. at −73° C. and was then warmed to 3° C. and stirred for 20 min. at this temperature. A solution of 1,3-diiodopropane (54 ml, 0.466 mol, 2.5 eq.) in DMPU (110 ml) was added dropwise over 20 min. keeping the internal temperature below 9° C. The reaction mixture was stirred for 20 min. and was then quenched by the addition of pH=7 phosphate buffer (300 ml, 0.5 M) and water (500 ml). The aqueous mixture was extracted with Et₂O (5×300 ml), the ether layer was washed with Na₂S₂O₃ solution (200 ml, 1 M), brine (2×200 ml), dried over Na₂SO₄ and concentrated. The crude product was purified by flash chromatography on silica using PtEt/EtOAc-4:1 as eluents to produce 16 g 3-Isobutoxy-4,5,6,6a-tetrahydro-3aH-pentalen-1-one.
Step 2) 2.0 g 3-Isobutoxy-4,5,6,6a-tetrahydro-3aH-pentalen-1-one was dissolved in 40 ml DCM. 2.47 g Br₂ was added drop wise and the mixture was stirred at rt for 20 min. The reaction mixture was cooled to 0° C. and 3.12 g TEA was slowly added. The cooling bath was removed and the mixture was stirred at rt for 1 h. Filtration and concentration gave a crude product which was purified on silica using 2% MeOH in DCM to produce 2.26 g 1-Bromo-2,3-difluoro-4-methoxy-benzene.
Step 3) n-BuLi (1.1 eq) was added to a solution of 40 mg 1-Bromo-2,3-difluoro-4-methoxy-benzene in 5 ml dry ether at −78° C. under N₂. 440 mg (1.61 mmol) 2-Bromo-3-isobutoxy-4,5,6,6a-tetrahydro-3aH-pentalen-1-one dissolved in 5 ml dry ether and cooled to −78° C. was added drop wise. The mixture was stirred under N₂ and the temperature was slowly raised overnight. The reaction was quenched with 10 ml water followed by 10 ml HCl (2N). The aqueous mixture was extracted with ether 3× and the combined organic layers were washed with brine and dried over Na₂SO₄. Concentration followed by purification on silica using a ether/n-heptane (1:9-2:8) gradient afforded 390 mg 2-Bromo-3-(2,3-difluoro-4-methoxy-phenyl)-4,5,6,6a-tetrahydro-3aH-pentalen-1-one as a clear oil which solidified after a while.
Step 4) 50 mg 2-Bromo-3-(2,3-difluoro-4-methoxy-phenyl)-4,5,6,6a-tetrahydro-3aH-pentalen-1-one was dissolved in 2 ml dry DCM under N₂ and the solution was cooled to −78° C. BBr3 (5 eq) was added dropwise, the cooling bath was removed and the mixture was stirred over night. The reaction was quenched by adding 200 μl MeOH at 0° C. followed by the addition of NaHCO₃ (sat, aq). The mixture was then allowed to reach n and was extracted with DCM 3× using a phase separator. The combined organic layers were concentrated and the crude product was purified on silica using a EtOAc/n-heptane (3:7-4:6) gradient. 44.8 mg 2-Bromo-3-(2,3-difluoro-4-hydroxy-phenyl)-4,5,6,6a-tetrahydro-3aH-pentalen-1-one was obtained. ES/MS m/z: 329.0 (pos. M+H), 327.1 (neg. M−H); ¹H NMR (CD₃OD, 500 MHz): d 7.22 (m, 1H), 6.85 (m, 1H), 3.88 (m, 1H), 3.06 (m, 1H), 1.91-1.85 (m, 2H), 1.75-1.60 (m, 2H), 1.48 (m, 1H) and 1.29 (m, 1H).

### Examples 81-131

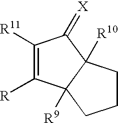

The following compounds were prepared in like manner to the preceding examples (if not stated otherwise R⁹ and R¹⁰ are hydrogen):

| | |
|---|---|
| E 81 | 3-(2,3-Difluoro-4-hydroxy-phenyl)-1-oxo-1,3a,4,5,6,6a-hexahydro-pentalene-2- |
| | carbonitrile |
| R¹¹ = CN | R = 2,3-Difluoro-4-hydroxy-phenyl |

ES/MS m/z: 275.9 (pos. M + H), 274.3 (neg. M − H); ¹H NMR (CD₃OD, 500 MHz): d 7.59 (m, 1H),
6.92 (m, 1H), 4.16 (m, 1H), 3.07 (m, 1H), 1.96-1.85 (m, 3H), 1.67 (m, 1H), 1.55 (m, 1H), 1.29 (m, 1H).

| | |
|---|---|
| E 82 | 2-Bromo-3-(2,5-difluoro-4-hydroxy-phenyl)-4,5,6,6a-tetrahydro-3aH-pentalen-1-one |
| R¹¹ = Br | R = 2,5-difluoro-4-hydroxy-phenyl |

ES/MS m/z: 329.06 (pos. M + H); ¹H NMR (CDCl₃, 500 MHz): d 7.36 (dd, J = 10.72, 6.31 Hz, 1H),
6.86 (dd, J = 11.03, 7.25 Hz, 1H), 3.85 (m, 1H), 3.05 (m, 1H), 1.97 (m, 1H), 1.85 (m, 1H), 1.67 (m, 2H),
1.51 (m, 1H) and 1.32 (m, 1H)

| | |
|---|---|
| E 83 | 3-(3-Fluoro-4-hydroxy-phenyl)-1-oxo-1,3a,4,5,6,6a-hexahydro-pentalene-2-carbonitrile |
| R¹¹ = CN | R = 3-Fluoro-4-hydroxy-phenyl |

ES/MS m/z: 258.08 (pos. M + H),; ¹H NMR (CD₃CN, 500 MHz): d 7.82 (m, 2H), 7.17 (m, 1H), 3.99 (m,
1H), 3.01 (m, 1H), 2.02 (m, 1H), 1.86 (m, 2H), 1.64 (m, 1H), 1.56 (m, 1H) and 1.32 (m, 1H).

| | |
|---|---|
| E 84 | 2-Bromo-3-(3-fluoro-4-hydroxy-phenyl)-6a-methyl-4,5,6,6a-tetrahydro-3aH-pentalen-1- |
| | one |
| R¹¹ = Br | R = 3-Fluoro-4-hydroxy-phenyl |
| R¹⁰ = CH₃ | |

ES/MS m/z: 325.04 (pos. M + H); ¹H NMR (CD₃OD, 500 MHz): d 7.82 (dd, J = 12.61, 2.21 Hz, 1H),
7.70 (m, 1H), 7.05 (m, 1H), 3.51 (dd, J = 9.14, 1.89 Hz, 1H), 1.98 (m, 1H), 1.93 (m, 1H), 1.66 (m, 1H),
1.51 (m, 2H), 1.29 (s, 3H) and 1.25 (m, 1H).

| | |
|---|---|
| E 85 | 3-(3-Fluoro-4-hydroxy-phenyl)-6a-methyl-1-oxo-1,3a,4,5,6,6a-hexahydro-pentalene-2- |
| | carbonitrile |
| R¹¹ = CN | R = 3-Fluoro-4-hydroxy-phenyl |
| R¹⁰ = CH₃ | |

ES/MS m/z: 272.07 (pos. M + H); ¹H NMR (CD₃OD, 500 MHz): d 7.87 (m, 2H), 7.10 (m, 1H), 3.66 (dd,
J = 9.80, 2.45 Hz, 1H), 2.20 (m, 1H), 1.97 (m, 1H), 1.71 (m, 1H), 1.63 (m, 1H), 1.53 (m, 1H), 1.33 (m,
1H) and 1.29 (s, 3H).

| | |
|---|---|
| E 86 | 2-Bromo-3-(2,3-difluoro-4-hydroxy-phenyl)-6a-methyl-4,5,6,6a-tetrahydro-3aH- |
| | pentalen-1-one |
| R¹¹ = Br | R = 2,3-Difluoro-4-hydroxy-phenyl |
| R¹⁰ = CH₃ | |

ES/MS m/z: 343.1 (pos. M + H), 341.2 (neg. M − H); ¹H NMR (CD₃OD, 500 MHz): d 7.22 (m, 1H),
6.85 (m, 1H), 3.43 (dt J = 8.83, 1.58 Hz, 1H), 1.93 (ddt J = 12.93, 6.31, 1.58), 1.81 (m, 1H), 1.66 (m, 1H),
1.52 (m, 1H), 1.46 (m, 1H), 1.36-1.27 (m, 1H) and 1.31 (s, 3H).

| | |
|---|---|
| E 87 | 2-Bromo-3-(3,5-difluoro-4-hydroxy-phenyl)-4,5,6,6a-tetrahydro-3aH-pentalen-1-one |
| R¹¹ = Br | R = 3,5-Difluoro-4-hydroxy-phenyl |

ES/MS m/z: 329.03 (pos. M + H), 327.08 (neg. M − H); ¹H NMR (CDCl₃, 500 MHz): d 7.54-7.64 (m,
2H), 6.50 (s, 1H), 3.72-3.80 (m, 1H), 3.03-3.10 (m, 1H), 1.97-2.04 (m, 1H), 1.79-1.91 (m, 2H),
1.62-1.72 (m, 1H), 1.52-1.60 (m, 1H) and 1.27-1.39 (m, 1H).

| | |
|---|---|
| E 88 | 3-(2,3-Difluoro-4-hydroxy-phenyl)-6a-methyl-1-oxo-1,3a,4,5,6,6a-hexahydro-pentalene-2- |
| | carbonitrile |
| R¹¹ = CN | R = 2,3-Difluoro-4-hydroxy-phenyl |
| R¹⁰ = CH₃ | |

ES/MS m/z: 290.3 (pos. M + H), 287.8 (neg. M − H); ¹H NMR (CD₃OD, 500 MHz): d 7.59 (m, 1H),
6.92 (m, 1H), 3.72 (dt J = 9.46, 2.21, 1H), 2.06-1.93 (m, 2H), 1.69 (m, 1H), 1.59-1.51 (m, 2H),
1.36-1.24 (m, 1H) and 1.31 (m, 1H).

| | |
|---|---|
| E 89 | 3-(3-Chloro-5-fluoro-4-hydroxy-phenyl)-1-oxo-1,3a,4,5,6,6a-hexahydro-pentalene-2- |
| | carbonitrile |
| R¹¹ = CN | R = 3-Chloro-5-fluoro-4-hydroxy-phenyl |

ES/MS m/z: 292.05 (pos. M + H), 290.9 (neg. M − H); ¹H NMR (CDCl₃, 500 MHz): d 7.86 (t, J = 2.2 Hz,
1H), 7.80 (dd, J = 10.8, 2.2 Hz, 1H), 6.66 (br s, 1H), 3.85-3.92 (m, 1H), 3.05-3.12 (m, 1H),
1.99-2.11 (m, 2H), 1.84-1.95 (m, 1H), 1.62-1.78 (m, 2H) and 1.29-1.41 (m, 1H).

| | |
|---|---|
| E 90 | 3-(3,5-Difluoro-4-hydroxy-phenyl)-3a-hydroxy-1-oxo-1,3a,4,5,6,6a-hexahydro-pentalene- |
| | 2-carbonitrile |
| R¹¹ = CN | R = 3,5-Difluoro-4-hydroxy-phenyl |
| R⁹ = OH | |

ES/MS m/z: 292.13 (pos. M + H), 290.13 (neg. M − H); ¹H NMR (d6-Acetone, 500 MHz): d
7.98-8.09 (m, 2H), 2.87 (dd, J = 9.7, 3.1 Hz, 1H), 2.12-2.22 (m, 2H), 2.06-2.11 (m, 1H), 1.85-1.93 (m, 1H),
1.77-1.85 (m, 1H) and 1.41-1.53 (m, 1H).

| | |
|---|---|
| E 91 | 3-(3,5-Difluoro-4-hydroxy-phenyl)-4,5,6,6a-tetrahydro-3aH-pentalen-1-one |
| R¹¹ = H | R = 3,5-Difluoro-4-hydroxy-phenyl |

ES/MS m/z: 251.07 (pos. M + H), 249.1 (neg. M − H); ¹H NMR (CDCl₃, 500 MHz): d 7.21-7.25 (m,
2H), 6.39 (d, J = 0.9 Hz, 1H), 5.75 (br s, 1H), 3.65-3.71 (m, 1H), 2.94-3.01 (m, 1H), 1.96-2.04 (m, 1H),
1.84-1.96 (m, 1H), 1.72-1.83 (m, 1H), 1.62-1.71 (m, 2H) and 1.26-1.37 (m, 1H).

| | |
|---|---|
| E 92 | 3-(3,5-Difluoro-4-hydroxy-phenyl)-1-oxo-1,3a,4,5,6,6a-hexahydro-pentalene-2- |
| | carbonitrile |
| R¹¹ = CN | R = 3,5-Difluoro-4-hydroxy-phenyl |

ES/MS m/z: 276.07 (pos. M + H), 274.08 (neg. M − H); ¹H NMR (d6-Acetone, 500 MHz): d
7.77-7.87 (m, 2H), 4.14-4.21 (m, 1H), 3.05-3.12 (m, 1H), 2.08-2.17 (m, 1H), 1.82-1.94 (m, 2H), 1.62-1.72 (m,
2H) and 1.31-1.42 (m, 1H).

| | |
|---|---|
| E 93 | 3-(2,5-Difluoro-4-hydroxy-phenyl)-1-oxo-1,3a,4,5,6,6a-hexahydro-pentalene-2- |
| | carbonitrile |
| R¹¹ = CN | R = 2,5-Difluoro-4-hydroxy-phenyl |

ES/MS m/z: 276.08 (pos. M + H); ¹H NMR (CD₃CN, 500 MHz): d 7.58 (dd, J = 11.66, 6.62 Hz, 1H),
6.94 (dd, J = 11.98, 7.25 Hz, 1H), 4.04 (m, 1H), 3.02 (m, 1H), 1.84 (m, 3H), 1.61 (m, 1H), 1.51 (m, 1H)
and 1.28 (m, 1H).

| | |
|---|---|
| E 94 | 2-Bromo-3-(2,5-difluoro-4-hydroxy-phenyl)-6a-methyl-4,5,6,6a-tetrahydro-3aH- |
| | pentalen-1-one |
| R¹¹ = Br | R = 2,5-Difluoro-4-hydroxy-phenyl |
| R¹⁰ = CH₃ | |

ES/MS m/z: 343 (pos. M + H), 341 (neg. M − H); ¹H NMR (d⁶-Acetone, 500 MHz): d 7.45 (dd, 1H,
J = 11.3, 6.7 Hz), 6.99 (dd, 1H, J = 11.3, 7.3 Hz), 3.48 (m, 1H), 1.98 (m, 1H), 1.87 (m, 1H), 1.72 (m, 1H),
1.59 (m, 1H), 1.53 (m, 1H), 1.41 (m, 1H) and 1.38 (s, 3H).

| | |
|---|---|
| E 95 | 3-(2,5-Difluoro-4-hydroxy-phenyl)-6a-methyl-1-oxo-1,3a,4,5,6,6a-hexahydro-pentalene-2- |
| | carbonitrile |
| R¹¹ = CN | R = 2,5-Difluoro-4-hydroxy-phenyl |
| R¹⁰ = CH₃ | |

ES/MS m/z: 290.1 (pos. M + H), 288.1 (neg. M − H)

| | |
|---|---|
| E 96 | 2,6a-Dibromo-3-(2,3-difluoro-4-hydroxy-phenyl)-4,5,6,6a-tetrahydro-3aH-pentalen-1-one |
| R¹¹ = Br | R = 2,3-Difluoro-4-hydroxy-phenyl |
| R¹⁰ = Br | |

ES/MS m/z: 407.0/409.1/410.9 (pos. M + H), 405.4/407.2/409.3 (neg. M − H); ¹H NMR (CD3OD,
500 MHz): d 7.32 (m, 1H), 6.87 (m, 1H), 4.08 (m, 1H), 2.39 (m, 1H), 2.21 (m, 1H), 2.06 (m, 1H),
1.79 (m, 1H), 1.50 (m, 1H) and 1.35 (m, 1H).

| | |
|---|---|
| E 97 | 2-Bromo-3-(3,5-difluoro-4-hydroxy-phenyl)-4,5,6,6a-tetrahydro-3aH-pentalen-1-one |
| | oxime |
| R¹¹ = Br | R = 3,5-Difluoro-4-hydroxy-phenyl |
| X = NOH | |

ES/MS m/z: 344.01/346.04 (pos. M + H), 342.05/344.05 (neg. M − H); ¹H NMR (acetone-d6,
500 MHz): d 7.43 (m, 2H), 3.83 (m, 1H), 3.53 (m, 1H), 2.03-1.95 (m, 2H), 1.87 (m, 1H), 1.81 (m, 1H),
1.58 (m, 1H) and 1.43 (m, 1H).

| | |
|---|---|
| E 98 | 2-Bromo-3-(3,5-difluoro-4-hydroxy-4-hydroxy-phenyl)-4,5,6,6a-tetrahydro-3aH-pentalen-1-one |
| | oxime |
| R¹¹ = CN | R = 3,5-Difluoro-4-hydroxy-phenyl |
| X = NOH | |

ES/MS m/z: 291.09 (pos. M + H), 289.11 (neg. M − H); ¹H NMR (acetone-d6, 500 MHz): d 7.59 (m,
2H), 4.03 (m, 1H), 3.58 (m, 1H), 2.04-1.98 (m, 2H), 1.91 (m, 1H), 1.62 (m, 1H) and 1.57-1.44 (m, 2H).

| | |
|---|---|
| E 99 | 2-Bromo-3-(3,5-difluoro-4-hydroxy-phenyl)-6a-methyl-4,5,6,6a-tetrahydro-3aH- |
| | pentalen-1-one |
| R¹¹ = Br | R = 3,5-Difluoro-4-hydroxy-phenyl |
| R¹⁰ = Me | |

ES/MS m/z: 342.97/344.99 (pos. M + H), 341.03/343.03 (neg. M − H); ¹H NMR (acetone-d6,
500 MHz): d 7.68 (m, 2H), 3.59 (dd, 1H, J = 9.2, 2.1 Hz), 1.99 (m, 1H), 1.90 (m, 1H), 1.64 (m, 1H),
1.54-1.47 (m, 2H), 1.29 (m, 1H) and 1.28 (s, 3H).

| | |
|---|---|
| E 100 | 3-(3,5-Difluoro-4-hydroxy-phenyl)-6a-methyl-1-oxo-1,3a,4,5,6,6a-hexahydro-pentalene-2- |
| | carbonitrile |
| R¹¹ = CN | R = 3,5-Difluoro-4-hydroxy-phenyl |
| R¹⁰ = Me | |

ES/MS m/z: 290.09 (pos. M + H), 288.13 (neg. M − H); ¹H NMR (acetone-d6, 500 MHz): d 7.80 (m,
2H), 3.76 (dd, 1H, J = 9.8, 2.5 Hz), 2.21 (m, 1H), 1.95 (m, 1H), 1.72-1.62 (m, 2H), 1.54 (m, 1H), 1.36 (m,
1H) and 1.29 (s, 3H).

| | |
|---|---|
| E 101 | 2-Bromo-3-(3,5-difluoro-4-hydroxy-phenyl)-6a-methyl-4,5,6,6a-tetrahydro-3aH- |
| | pentalen-1-one oxime |
| R¹¹ = Br | R = 3,5-Difluoro-4-hydroxy-phenyl |
| R¹⁰ = Me | X = NOH |

ES/MS m/z: 358.07/360.05 (pos. M + H), 356.11/358.06 (neg. M − H); ¹H NMR (acetone-d6,
500 MHz): d 7.46 (m, 2H), 3.30 (dd, 1H, J = 8.7, 4.2 Hz), 2.38 (m, 1H), 1.94 (m, 1H), 1.71-1.63 (m, 2H),
1.53 (s, 3H), 1.50 (m, 1H) and 1.38 (m, 1H).

| | |
|---|---|
| E 102 | 3-(3-Chloro-4-hydroxy-phenyl)-1-oxo-1,3a,4,5,6,6a-hexahydro-pentalene-2-carbonitrile |
| R¹¹ = CN | R = 3-Chloro-4-hydroxy-phenyl |

ES/MS m/z: 274.1 (pos. M + H), 272.1 (neg. M − H); ¹H NMR (CD₃OD, 500 MHz): d 8.12 (d, 1H,
J = 2.4 Hz), 7.97 (dd, 1H, J = 8.6, 2.4 Hz), 7.09 (d, 1H, J = 8.6 Hz), 4.09 (m, 1H), 3.06 (m, 1H), 2.08 (m, 1H),
1.96-1.84 (m, 2H), 1.69 (m, 1H), 1.61 (m, 1H) and 1.32 (m, 1H).

| | |
|---|---|
| E 103 | 2-Bromo-3-(3-chloro-5-fluoro-4-hydroxy-phenyl)-6a-methyl-4,5,6,6a-tetrahydro-3aH- |
| | pentalen-1-one |
| R¹¹ = Br | R = 3-chloro-5-fluoro-4-hydroxy-phenyl |
| R¹⁰ = Me | |

ES/MS m/z: 359/361 (pos. M + H), 357/359 (neg. M − H); ¹H NMR (acetone-d6, 500 MHz): d 7.86 (br
s, 1H), 7.78 (dd, 1H, J = 11.8, 1.9 Hz), 3.59 (dd, 1H, J = 9.2, 2.1 Hz), 1.99 (m, 1H), 1.90 (m, 1H), 1.65 (m,
1H), 1.55-1.47 (m, 2H), 1.30 (m, 1H) and 1.28 (s, 3H).

| | |
|---|---|
| E 104 | 3-(3-Chloro-5-fluoro-4-hydroxy-phenyl)-6a-methyl-1-oxo-1,3a,4,5,6,6a-hexahydro- |
| | pentalene-2-carbonitrile |
| R¹¹ = CN | R = 3-chloro-5-fluoro-4-hydroxy-phenyl |
| R¹⁰ = Me | |

ES/MS m/z: 306.04 (pos. M + H), 304.07 (neg. M − H); ¹H NMR (acetone-d6, 500 MHz): d 8.00 (t, 1H,
J = 1.5 Hz), 7.89, (dd, 1H, J = 11.7, 2.2 Hz), 3.78 (dd, 1H, J = 9.9, 2.6 Hz), 2.20 (m, 1H), 1.95 (m, 1H),
1.72-1.62 (m, 2H), 1.53 (m, 1H), 1.36 (m, 1H) and 1.29 (s, 3H).

| | |
|---|---|
| E 105 | 2-Bromo-3-(2-chloro-4-hydroxy-phenyl)-4,5,6,6a-tetrahydro-3aH-pentalen-1-one |
| R¹¹ = Br | R = 2-chloro-4-hydroxy-phenyl |

ES/MS m/z: 329 (pos. M + H), 327 (neg. M − H); ¹H NMR (CD₃OD, 500 MHz): d 7.15 (d, 1H,
J = 8.5 Hz), 6.94 (d, 1H, J = 2.3 Hz), 6.84 (dd, 1H, J = 8.5, 2.3 Hz), 3.82 (m, 1H), 3.05 (m, 1H), 1.95-1.82 (m,
2H), 1.68-1.56 (m, 3H) and 1.38 (m, 1H).

| | |
|---|---|
| E 106 | 3-(2-Fluoro-4-hydroxy-phenyl)-1-oxo-1,3a,4,5,6,6a-hexahydro-pentalene-2-carbonitrile |
| R¹¹ = CN | R = 2-fluoro-4-hydroxy-phenyl |

ES/MS m/z: 258.1 (pos. M + H), 256.1 (neg. M − H); ¹H NMR (CD₃OD, 500 MHz): d 7.82 (t, 1H,
J = 8.5 Hz), 6.80 (dd, 1H, J = 8.5, 2.4 Hz), 6.70 (dd, 1H, J = 13.4, 2.4 Hz), 4.17 (m, 1H), 3.05 (m, 1H),
1.94-1.84 (m, 3H), 1.65 (m, 1H), 1.55 (m, 1H) and 1.28 (m, 1H).

| | |
|---|---|
| E 107 | 2-Bromo-3-(5-chloro-2,3-difluoro-4-hydroxy-phenyl)-4,5,6,6a-tetrahydro-3aH-pentalen- |
| | 1-one |
| R¹¹ = Br | R = 5-chloro-2,3-difluoro-4-hydroxy-phenyl |

ES/MS m/z: 364.9 (pos. M + H), 363 (neg. M − H); ¹H NMR (CD₃OD, 500 MHz): d 7.40 (dd, 1H,
J = 7.0, 2.5 Hz), 3.85 (m, 1H), 3.07 (m, 1H), 1.90-1.86 (m, 2H), 1.75-1.62 (m, 2H), 1.49 (m, 1H) and
1.30 (m, 1H).

| | |
|---|---|
| E 108 | 2-Bromo-3-(2,3-dichloro-4-hydroxy-phenyl)-4,5,6,6a-tetrahydro-3aH-pentalen-1-one |
| R¹¹ = Br | R = 2,3-dichloro-4-hydroxy-phenyl |

ES/MS m/z: 360.96/362.98/364.94 (pos. M + H), 358.97/360.95/362.96 (neg. M − H); ¹H NMR
(CDCl₃, 500 MHz): d 7.08 (d, 1H, J = 8.6 Hz), 7.04 (d, 1H, J = 8.6 Hz), 3.75 (m, 1H), 3.08 (m, 1H),
2.05 (m, 1H), 1.85 (m, 1H), 1.68 (m, 1H), 1.60-1.56 (m, 2H) and 1.40 (m, 1H).

| | |
|---|---|
| E 109 | 3-(2,3-Dichloro-4-hydroxy-phenyl)-4,5,6,6a-tetrahydro-3aH-pentalen-1-one |
| R¹¹ = H | R = 2,3-Dichloro-4-hydroxy-phenyl |

ES/MS m/z: 283.02/285.01 (pos. M + H), 281.07/283.07 (neg. M − H); ¹H NMR (acetone-d6,
500 MHz): d 7.41 (d, 1H, J = 8.5 Hz), 7.12 (d, 1H, J = 8.5 Hz), 3.98 (m, 1H), 2.84 (m, 1H), 1.85 (m, 1H),
1.77 (m, 1H), 1.66 (m, 1H), 1.59 (m, 1H) 1.46 (m, 1H) and 1.29 (m, 1H).

| | |
|---|---|
| E 110 | 3-(5-Chloro-2,3-difluoro-4-hydroxy-phenyl)-1-oxo-1,3a,4,5,6,6a-hexahydro-pentalene-2- |
| | carbonitrile |
| R¹¹ = CN | R = 5-Chloro-2,3-difluoro-4-hydroxy-phenyl |

ES/MS m/z: 310 (pos. M + H), 308.1 (neg. M − H); ¹H NMR (CD₃OD, 500 MHz): d 7.75 (dd, 1H,
J = 6.9, 2.2 Hz), 4.14 (m, 1H), 3.08 (m, 1H), 1.94-1.86 (m, 3H), 1.68 (m, 1H), 1.55 (m, 1H) and 1.32 (m,
1H).

| | |
|---|---|
| E 111 | 3-(2-Chloro-4-hydroxy-phenyl)-1-oxo-1,3a,4,5,6,6a-hexahydro-pentalene-2-carbonitrile |
| R¹¹ = CN | R = 2-Chloro-4-hydroxy-phenyl |

ES/MS m/z: 274 (pos. M + H), 272 (neg. M − H); ¹H NMR (CD₃OD, 500 MHz): d 7.41 (d, 1H,
J = 8.5 Hz), 7.01 (d, 1H, J = 2.5 Hz), 6.89 (dd, 1H, J = 8.5, 2.5 Hz), 4.16 (m, 1H), 3.08 (m, 1H), 1.96-1.85 (m,
2H), 1.75 (m, 1H), 1.66 (m, 1H), 1.51 (m, 1H) and 1.33 (m, 1H).

| | |
|---|---|
| E 112 | 2-Bromo-3-(2-chloro-3-fluoro-4-hydroxy-phenyl)-4,5,6,6a-tetrahydro-3aH-pentalen-1- |
| | one |
| R¹¹ = Br | R = 2-chloro-3-fluoro-4-hydroxy-phenyl |

ES/MS m/z: 345/347 (pos. M + H), 343/345 (neg. M − H); ¹H NMR (acetone-d6, 500 MHz); d
7.15-7.09 (m, 2H), 3.82 (m, 1H), 3.06 (m, 1H), 3.05 (m, 1H), 1.92-1.81 (m, 2H), 1.68-1.56 (m, 3H) and 1.40 (m,
1H).

| | |
|---|---|
| E 113 | 3-(5-Chloro-2-fluoro-4-hydroxy-phenyl)-1-oxo-1,3a,4,5,6,6a-hexahydro-pentalene-2- |
| | carbonitrile |
| R¹¹ = Br | R = 5-Chloro-2-fluoro-4-hydroxy-phenyl |

ES/MS m/z: 292.1 (pos. M + H), 290.1 (neg. M − H); ¹H NMR (CD₃OD, 500 MHz): d 7.93 (d, 1H,
J = 7.6 Hz), 6.82 (d, 1H, J = 12.6 Hz), 4.15 (m, 1H), 3.05 (m, 1H), 1.95-1.85 (m, 3H), 1.67 (m, 1H),
1.55 (m, 1H) and 1.30

| | |
|---|---|
| E 114 | 2-Bromo-3-(5-bromo-2-chloro-3-fluoro-4-hydroxy-phenyl)-4,5,6,6a-tetrahydro-3aH- |
| | pentalen-1-one |
| R¹¹ = Br | R = 5-bromo-2-chloro-3-fluoro-4-hydroxy-phenyl |

ES/MS m/z: 422.96/425.01/427.02 (pos. M + H), 420.75/422.76/424.73 (neg. M − H); ¹H NMR
(CDCl₃, 500 MHz): d 7.39 (d, 1H, J = 2.2 Hz), 3.91 (m, 1H), 3.07 (m, 1H), 2.00 (m, 1H), 1.85 (m, 1H),
1.69-1.59 (m, 2H), 1.51 (m, 1H) and 1.35 (m, 1H).

| | |
|---|---|
| E 115 | 3-(5-Chloro-2-fluoro-4-hydroxy-phenyl)-1-oxo-1,3a,4,5,6,6a-hexahydro-pentalene-2- |
| | carbonitrile |
| R¹¹ = CN | R = 5-Chloro-2-fluoro-4-hydroxy-phenyl |

ES/MS m/z: 292.1 (pos. M + H), 290.1 (neg. M − H); ¹H NMR (CD₃OD, 500 MHz): d 7.93 (d, 1H,
J = 7.6 Hz), 6.82 (d, 1H, J = 12.6 Hz), 4.15 (m, 1H), 3.05 (m, 1H), 1.95-1.85 (m, 3H), 1.67 (m, 1H),
1.55 (m, 1H) and 1.30 (m, 1H).

| | |
|---|---|
| E 116 | 3-(2-Chloro-3-fluoro-4-hydroxy-phenyl)-4,5,6,6a-tetrahydro-3aH-pentalen-1-one |
| R¹¹ = H | R = 2-Chloro-3-fluoro-4-hydroxy-phenyl |

ES/MS m/z: 267.08 (pos. M + H), 265.05 (neg. M − H); ¹H NMR (acetone-d6, 500 MHz): d 7.34 (dd,
1H, J = 8.8, 1.9 Hz), 7.10 (t, 1H, J = 8.8 Hz), 6.43 (d, 1H, J = 1.2 Hz), 3.99 (m, 1H), 2.83 (m, 1H), 1.85 (m,
1H), 1.80-1.67 (m, 2H), 1.59 (m, 1H), 1.46 (m, 1H) and 1.27 (m, 1H).

| | |
|---|---|
| E 117 | 3-(2-Chloro-3-fluoro-4-hydroxy-phenyl)-1-oxo-1,3a,4,5,6,6a-hexahydro-pentalene-2- |
| | carbonitrile |
| R¹¹ = CN | R = 2-Chloro-3-fluoro-4-hydroxy-phenyl |

ES/MS m/z: 292.07 (pos. M + H), 290.09 (neg. M − H); ¹H NMR (acetone-d6, 500 MHz): d 7.35 (dd,
1H, J = 8.8, 1.9 Hz), 7.18 (t, 1H, J = 8.8 Hz), 4.19 (m, 1H), 3.13 (m, 1H), 1.94-1.86 (m, 2H), 1.77 (m, 1H),
1.67 (m, 1H), 1.55 (m, 1H) and 1.38 (m, 1H).

| | |
|---|---|
| E 118 | 2-Bromo-3-(2,6-difluoro-4-hydroxy-phenyl)-4,5,6,6a-tetrahydro-3aH-pentalen-1-one |
| R¹¹ = Br | R = 2,6-difluoro-4-hydroxy-phenyl |

ES/MS m/z: 329 (pos. M + H), 327 (neg. M − H); ¹H NMR (CD₃OD, 500 MHz): d 6.51 (m, 2H),
3.76 (m, 1H), 3.06 (m, 1H), 1.92-1.82 (m, 2H), 1.70-1.62 (m, 2H), 1.51 (m, 1H) and 1.30 (m, 1H).

| | |
|---|---|
| E 119 | 3-(2,6-Difluoro-4-hydroxy-phenyl)-1-oxo-1,3a,4,5,6,6a-hexahydro-pentalene-2- |
| | carbonitrile |
| R¹¹ = CN | R = 2,6-Difluoro-4-hydroxy-phenyl |

ES/MS m/z: 276.1 (pos. M + H), 274.1 (neg. M − H); ¹H NMR (CD₃OD, 500 MHz): d 6.59 (m, 2H),
4.12 (m, 1H), 3.08 (m, 1H), 1.95-1.88 (m, 2H), 1.80 (m, 1H), 1.67 (m, 1H), 1.52 (m, 1H) and 1.25 (m,
1H).

| | |
|---|---|
| E 120 | 2-Bromo-3-(3-chloro-2-fluoro-4-hydroxy-phenyl)-4,5,6,6a-tetrahydro-3aH-pentalen-1- |
| | one |
| R¹¹ = Br | R = 3-chloro-2-fluoro-4-hydroxy-phenyl |

ES/MS m/z: 345/347 (pos. M + H), 343/345 (neg. M − H); ¹H NMR (acetone-d6, 500 MHz): d 7.43 (dd,
1H, J = 8.6, 7.9 Hz), 7.03 (dd, 1H, J = 8.6, 1.6 Hz), 3.89 (m, 1H), 3.05 (m, 1H), 1.88-1.83 (m, 2H),
1.70 (m, 1H), 1.63 (m, 1H), 1.49 (m, 1H) and 1.30 (m, 1H).

| | |
|---|---|
| E 121 | (3aS,6aR)-3-(2,3-Difluoro-4-hydroxy-phenyl)-1-oxo-1,3a,4,5,6,6a-hexahydro-pentalene-2- |
| | carbonitrile |
| R¹¹ = CN | R = 2,3-Difluoro-4-hydroxy-phenyl |

ES/MS m/z: 276.2 (pos. M + H), 274.3 (neg. M − H); ¹H NMR (CD₃OD, 500 MHz): d 7.62 (m, 1H),
6.86 (m, 1H), 4.16 (m, 1H), 3.06 (m, 1H), 1.97-1.85 (m, 3H), 1.67 (m, 1H), 1.57 (m, 1H) and 1.30 (m,
1H).

| | |
|---|---|
| E 122 | (3aR,6aS)-3-(2,3-Difluoro-4-hydroxy-phenyl)-1-oxo-1,3a,4,5,6,6a-hexahydro-pentalene-2- |
| | carbonitrile |
| R¹¹ = CN | R = 2,3-Difluoro-4-hydroxy-phenyl |

ES/MS m/z: 276.22 (pos. M + H), 274 (neg. M − H); ¹H NMR (CD₃OD, 500 MHz): d 7.63 (m, 1H),
6.82 (m, 1H), 4.16 (m, 1H), 3.05 (m, 1H), 1.95-1.85 (m, 3H), 1.67 (m, 1H), 1.58 (m, 1H) and 1.29 (m, 1H).

| | |
|---|---|
| E 123 | 3-(3-Chloro-2-fluoro-4-hydroxy-phenyl)-1-oxo-1,3a,4,5,6,6a-hexahydro-pentalene-2- |
| | carbonitrile |
| R¹¹ = CN | R = 3-Chloro-2-fluoro-4-hydroxy-phenyl |

ES/MS m/z: 292.07 (pos. M + H), 292.09 (neg. M − H); ¹H NMR (acetone-d6, 500 MHz): d 7.77 (t, 1H,
J = 8.5 Hz), 7.09 (t, 1H, J = 8.5, 1.6 Hz), 4.21 (m, 1H), 3.08 (m, 1H), 1.94-1.86 (m, 3H), 1.65 (m, 1H),
1.57 (m, 1H) and 1.32 (m, 1H).

| | |
|---|---|
| E 124 | 2-Bromo-3-(2,3,5-trifluoro-4-hydroxy-phenyl)-4,5,6,6a-tetrahydro-3aH-pentalen-1-one |
| R¹¹ = Br | R = 2,3,5-trifluoro-4-hydroxy-phenyl |

ES/MS m/z: 347 (pos. M + H), 345.1 (neg. M − H); ¹H NMR (CD₃OD, 500 MHz): d 7.25 (m, 1H),
3.87 (m, 1H), 3.06 (m, 1H), 1.90-1.85 (m, 2H), 1.76-1.62 (m, 2H), 1.50 (m, 1H) and 1.29 (m, 1H).

| | |
|---|---|
| E 125 | 2-Bromo-3-(3-chloro-2,5-difluoro-4-hydroxy-phenyl)-4,5,6,6a-tetrahydro-3aH-pentalen- |
| | 1-one |
| R¹¹ = Br | R = 3-chloro-2,5-difluoro-4-hydroxy-phenyl |

ES/MS m/z: 365 (pos. M + H), 363 (neg. M − H); ¹H NMR (CD₃OD, 500 MHz): d 7.35 (dd, 1H, J = 11.0.
6.2 Hz), 3.85 (m, 1H), 3.07 (m, 1H), 1.90-1.86 (m, 2H), 1.74-1.62 (m, 2H), 1.48 (m, 1H) and 1.31 (m,
1H).

| | |
|---|---|
| E 126 | 3-(3-Chloro-2,5-difluoro-4-hydroxy-phenyl)-1-oxo-1,3a,4,5,6,6a-hexahydro-pentalene-2- |
| | carbonitrile |
| R¹¹ = CN | R = 3-Chloro-2,5-difluoro-4-hydroxy-phenyl |

ES/MS m/z: 310.4 (pos. M + H), 308.5 (neg. M − H); ¹H NMR (CD₃OD, 500 MHz): d 7.67 (dd, 1H,
J = 11.3, 6.5 Hz), 4.14 (m, 1H), 3.08 (m, 1H), 1.94-1.86 (m, 3H), 1.68 (m, 1H), 1.54 (m, 1H) and 1.31 (m,
1H).

| | |
|---|---|
| E 127 | 2-Bromo-3-(2,3,6-trifluoro-4-hydroxy-phenyl)-4,5,6,6a-tetrahydro-3aH-pentalen-1-one |
| R¹¹ = Br | R = 2,3,6-trifluoro-4-hydroxy-phenyl |

ES/MS m/z: 347.0/349.1 (pos. M + H), 344.8/347.2 (neg. M − H); ¹H NMR (CD₃OD, 500 MHz): d
6.67 (m, 1H), 3.77 (m, 1H), 3.08 (m, 1H), 1.92-1.83 (m, 2H), 1.71-1.64 (m, 2H), 1.53 (m, 1H) and
1.29 (m, 1H).

| | |
|---|---|
| E 128 | 2-Bromo-3-(4-hydroxy-phenyl)-6a-propyl-4,5,6,6a-tetrahydro-3aH-pentalen-1-one |
| R¹¹ = Br | R = 4-hydroxy-phenyl |
| R¹⁰ = Propyl | |

ES/MS m/z: 335.6/337.4 (pos. M + H), 333.4/335.5 (neg. M − H); ¹H NMR (acetone-d6, 500 MHz): d
7.97 (m, 2H), 7.00 (m, 2H), 3.69 (dd, 1H, J = 9.2, 1.9 Hz), 1.96-1.85 (m, 2H), 1.75 (m, 1H), 1.59 (m, 1H),
1.55-1.48 (m, 3H), 1.34 (m, 1H), 1.22 (m, 1H), 1.11 (m, 1H) and 0.86 (t, 3H, J = 7.1 Hz).

| | |
|---|---|
| E 129 | 3-(3,5-Difluoro-4-hydroxy-phenyl)-2-ethynyl-4,5,6,6a-tetrahydro-3aH-pentalen-1-one |
| R¹¹ = Ethynyl | R = 3,5-Difluoro-4-hydroxy-phenyl |

ES/MS m/z: 275.13 (pos. M + H), 273.17 (neg. M − H); ¹H NMR (acetone-d6, 500 MHz): d 7.92 (m,
2H), 4.23 (s, 1H), 3.97 (m, 1H), 2.94 (m, 1H), 2.01 (m, 1H), 1.89 (m, 1H), 1.81 (m, 1H), 1.66-1.60 (m,
2H) and 1.25 (m, 1H).

| | |
|---|---|
| E 130 | 3-(2,3-Difluoro-4-hydroxy-phenyl)-2-isopropenyl-4,5,6,6a-tetrahydro-3aH-pentalen-1- |
| | one |
| R¹¹ = Isopropenyl | R = 2,3-Difluoro-4-hydroxy-phenyl |

ES/MS m/z: 291.12 (pos. M + H), 289.18 (neg. M − H); ¹H NMR (acetone-d6, 500 MHz): d 7.10 (m, 1H),
6.92 (m, 1H), 4.99 (m, 1H), 4.78 (m, 1H), 3.72 (m, 1H), 2.89 (m, 1H), 1.89-1.75 (m, 5H), 1.65 (m, 1H),
1.58 (m, 1H), 1.46 (m, 1H) and 1.26 (m, 1H).

| | |
|---|---|
| E 131 | 3-(3,5-Difluoro-4-hydroxy-phenyl)-2-isopropenyl-4,5,6,6a-tetrahydro-3aH-pentalen-1- |
| | one |
| R¹¹ = Isopropenyl | R = 3,5-Difluoro-4-hydroxy-phenyl |

ES/MS m/z: 291.13 (pos. M + H), 289.18 (neg. M − H); ¹H NMR (acetone-d6, 500 MHz): d 7.33 (m,
2H), 5.20 (m, 1H), 4.88 (m, 1H), 3.81 (m, 1H), 2.85 (m, 1H), 1.90-1.73 (m, 6H), 1.58 (m, 1H), 1.50 (m,
1H) and 1.24 (m, 1H).

### Example 132

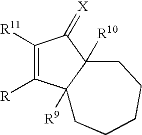

The following compound was prepared in like manner to the preceding examples:

| | |
|---|---|
| E 132 | 2-Bromo-3-(2,3-difluoro-4-hydroxy-phenyl)-4,5,6,7,8,8a-hexahydro-3aH-azulen-1-one |
| R¹¹ = Br | R = 3,5-Difluoro-4-hydroxy-phenyl |

ES/MS m/z: 357.16/359.14 (pos. M + H), 355.21/357.18 (neg. M − H); ¹H NMR (500 MHz): CDCl3)
d 7.12-7.17 (m, 1H), 6.90-6.95 (m, 1H), 5.62 (br s, 1H), 3.56-3.64 (m, 1H), 2.85-2.93 (m, 1H),
2.09-2.20 (m, 1H), 1.62-1.86 (m, 5H), 1.32-1.53 (m, 4H);

### Compounds 133-134

The following compounds were prepared in like manner to Examples 19-56 (if not stated otherwise R⁹ and R¹⁰ are hydrogen):

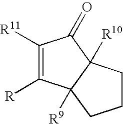

| Compound | 2-Bromo-3-(4-diallylamino-phenyl)-4,5,6,6a-tetrahydro-3aH-pentalen-1-one |
|---|---|
| 133 | |

| | |
|---|---|
| R¹¹ = Br | R = 4-diallylamino-phenyl |

¹H NMR (d6-Acetone, 500 MHz): d 8.06(m, 2H), 6.90(m, 2H), 6.02(m, 2H), 5.30(m, 2H), 5.27(m,
2H), 4.14(m, 4H), 4.00(m, 1H), 3.03(m, 1H), 1.99(m, 1H), 1.92(m, 2H), 1.71(m, 1H), 1.59(m, 1H)
and 1.38(m, 1H).

| | |
|---|---|
| Compound | 2-Bromo-3-(4-diallylamino-3-methyl-phenyl)-4,5,6,6a-tetrahydro-3aH-pentalen-1- |
| 134 | one |

| | |
|---|---|
| R¹¹ = Br | R = 4-diallylamino-3-methyl-phenyl |

ES/MS m/z: 386.1 (pos. M + H); ¹H NMR (CDCl₃, 500 MHz): d 7.79(d, J = 2.2 Hz, 1H), 7.42(dd, J = 2.2,
8.52 Hz, 1H), 7.06(d, J = 7.54 Hz, 1H), 5.81(m, 2H), 5.20(m, 4H), 3.83(m, 1H), 3.72(bs, 4H), 3.02(m,
1H), 2.40(s, 3H), 2.00(m, 1H), 1.82(m, 1H), 1.59(m, 2H) and 1.31(m, 1H).

### Compound 135

The following compound was prepared in like manner to Example 57:

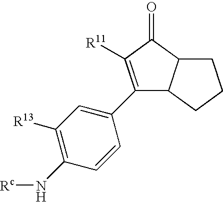

| Compound | |
|---|---|
| 135 | 3-(4-Allylamino-3-methyl-phenyl)-2-bromo-4,5,6,6a-tetrahydro-3aH-pentalen-1-one |

| | |
|---|---|
| R¹¹ = Br | R¹³ = Me |
| R^{c} = allyl | |

¹H NMR (CDCl₃, 500 MHz): d 7.85(dd, J = 2.2, 8.7 Hz, 1H), 7.82(d, J = 2.2 Hz, 1H), 6.67(d, J = 8.7 Hz,
1H), 5.98(m, 1H), 5.30(m, 1H), 5.23(m, 1H), 3.91(m, 2H), 3.82(m, 1H), 2.98(m, 1H), 2.22(s, 3H),
1.98(m, 1H), 1.87-1.76(m, 2H), 1.64-1.57(m, 2H) and 1.30(m, 1H)

### Compounds 136-139

The following compounds were prepared in like manner to the Examples 61 and 62:

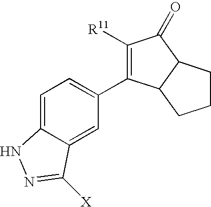

| Compound | 2-Chloro-3-(3-chloro-1H-indazol-5-yl)-4,5,6,6a-tetrahydro-3aH-pentalen-1-one |
|---|---|
| 136 | |

| | |
|---|---|
| R¹¹ = Cl | X = Cl |

ES/MS m/z: 307.0 (pos. M + H); (CDCl₃, 500 MHz): d 7.56(d, J = 1.6 Hz, 1H), 7.30(dd, J = 1.6, 9.0 Hz,
1H), 7.03(d, J = 9.0 Hz, 1H), 3.41(m, 1H), 2.29(m, 1H), 1.21-1.08(m, 3H), 0.89(m, 1H), 0.79(m, 1H)
and 0.58(m, 1H).

| | |
|---|---|
| Compound | 2-Bromo-3-(3-chloro-1H-indazol-5-yl)-4,5,6,6a-tetrahydro-3aH-pentalen-1-one |
| 137 | |

| | |
|---|---|
| R¹¹ = Br | X = Cl |

ES/MS m/z: 351.0 (pos. M + H); ¹H NMR (d6-Acetone, 500 MHz): d 8.30(d, J = 1.6 Hz, 1H), 8.02(dd,
J = 1.6, 8.9 Hz, 1H), 7.78(d, J = 8.9 Hz, 1H), 4.18(m, 1H), 3.07(m, 1H), 1.90-1.81(m, 3H), 1.63(m, 1H),
1.51(m, 1H) and 1.34(m, 1H).

| | |
|---|---|
| Compound | 3-(3-Bromo-1H-indazol-5-yl)-4,5,6,6a-tetrahydro-3aH-pentalen-1-one |
| 138 | |

| | |
|---|---|
| R¹¹ = H | X = Br |

ES/MS m/z: 317.0 (pos. M + H); (CDCl₃, 500 MHz): d 7.92(d, J = 1.4 Hz, 1H), 7.78(dd, J = 1.4, 8.9 Hz,
1H), 7.57(d, J = 8.9 Hz, 1H), 6.56(s, 1H), 3.92(m, 1H), 3.02(m, 1H), 2.05-1.95(m, 2H), 1.82, (m, 1H),
1.74-1.66(m, 2H) and 1.35(m ,1H).

| | |
|---|---|
| Compound | 2-Bromo-3-(3-bromo-1H-indazol-5-yl)-4,5,6,6a-tetrahydro-3aH-pentalen-1-one |
| 139 | |

| | |
|---|---|
| R¹¹ = Br | X = Br |

ES/MS m/z: 396.9 (pos. M + H); ); ¹H NMR (d6-Acetone, 500 MHz): d 7.72(d, J = 1.5 Hz, 1H) ,7.49(dd,
J = 1.5, 8.9 Hz, 1H), 7.29(d, J = 8.9 Hz, 1H), 3.70(m, 1H), 2.62(m, 1H), 1.41-1.28(m, 3H), 1.12(m, 1H),
0.90(m, 1H) and 0.78(m, 1H).

### Compound 140

Ethanesulfonic acid [4-(2-bromo-3-oxo-3,3a,4,5,6,6a-hexahydro-pentalen-1-yl)-phenyl]-amide

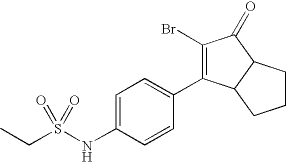

3-(4-Amino-phenyl)-2-bromo-4,5,6,6a-tetrahydro-3aH-pentalen-1-one (25 mg, 0.09 mmol) and N,N-diisopropylethylamine (33 mg, 0.26 mmol) were mixed in CH₂Cl₂ (0.5 ml, dry) at RT. To this suspension, the sulphonyl chloride (0.26 mmol) in CH₂Cl₂ (0.5 ml, dry) was added. The mixture was stirred at 40° C. over night. The mixture was washed with HCl 1M and the organic phase separated using a phase separator. Concentration and purification by preparative HPLC afforded 11 mg of the title compound. ES/MS m/z: 385.97 (pos. M+H), 381.99 (neg. M−H); ¹H NMR (d6-Acetone, 500 MHz): d 8.00 (m, 2H), 7.50 (m, 2H), 4.02 (m, 1H), 3.25 (m, 21-), 3.02 (m, 1H), 1.85 (m, 3H), 1.62 (m, 1H) 1.51 (m, 1H), 1.32 (m, 3H) and 1.28 (m, 1H).

### Compounds 141-147

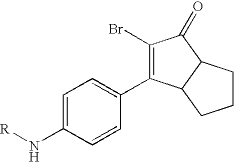

The following compounds were prepared in like manner to the preceding Compound:

| | |
|---|---|
| Compound | N-[4-(2-Bromo-3-oxo-3,3a,4,5,6,6a-hexahydro-pentalen-1-yl)-phenyl]- |
| 141 | methanesulfonamide |
| R = Methanesulfonyl | |

ES/MS m/z: 371.98 (pos. M + H), 369.96 (neg. M − H); ¹H NMR (d6-Acetone, 500 MHz): d 8.00 (m,
2H), 7.49 (m, 2H), 4.03 (m, 1H), 3.13 (s, 3H), 3.02 (m, 1H), 1.90-1.82 (m, 3H), 1.62 (m, 1H), 1.51 (m,
1H) and 1.27 (m, 1H).

| | |
|---|---|
| Compound | N-[4-(2-Bromo-3-oxo-3,3a,4,5,6,6a-hexahydro-pentalen-1-yl)-phenyl]-4-fluoro- |
| 142 | benzenesulfonamide |
| R = 4-Fluoro-benzenesulfonyl | |

ES/MS m/z: 451.94 (pos. M + H), 449.93 (neg. M − H); ¹H NMR (d6-Acetone, 500 MHz): d 7.98 (m,
2H), 7.91 (m, 2H), 7.39 (m, 2H), 7.34 (m, 2H), 3.97 (m, 1H), 2.99 (m, 1H), 1.85-1.77 (m, 3H), 1.59 (m,
1H), 1.42 (m, 1H) and 1.24 (m, 1H).

| | |
|---|---|
| Compound | N-[4-(2-Bromo-3-oxo-3,3a,4,5,6,6a-hexahydro-pentalen-1-yl)-phenyl]- |
| 143 | benzenesulfonamide |
| R = Benzenesulfonyl | |

ES/MS m/z: 433.96 (pos. M + H), 431.95 (neg. M − H); ¹H NMR (d6-Acetone, 500 MHz): d 8.06 (m,
2H), 8.04-8.00 (m, 4H), 7.60 (m, 1H), 7.53 (m, 2H), 4.05 (m, 1H), 3.03 (m, 1H), 1.92-1.82 (m, 3H),
1.63 (m, 1H), 1.54 (m, 1H) and 1.30 (m, 1H).

| | |
|---|---|
| Compound | Propane-1-sulfonic acid [4-(2-bromo-3-oxo-3,3a,4,5,6,6a-hexahydro-pentalen-1-yl)- |
| 144 | phenyl]-amide |
| R = Propanesulfonyl | |

ES/MS m/z: 433.96 (pos. M + H), 431.95 (neg. M − H); ¹H NMR (d6-Acetone, 500 MHz): d 8.00 (m,
2H), 7.49 (m, 2H), 4.03 (m, 1H), 3.22 (m, 2H), 3.02 (m, 1H), 1.90-1.79 (m, 5H), 1.62 (m, 1H), 1.51 (m,
1H), 1.27 (m, 1H) and 1.01 (t, J = 7.6 Hz, 3H).

| | |
|---|---|
| Compound | N-[4-(2-Bromo-3-oxo-3,3a,4,5,6,6a-hexahydro-pentalen-1-yl)-phenyl]-propionamide |
| 145 | |
| R = Propionyl | |

ES/MS m/z: 350.02 (pos. M + H), 348.04 (neg. M − H); ); ¹H NMR (d6-Acetone, 500 MHz): d 7.95 (m,
2H), 7.85 (m, 2H), 4.01 (m, 1H), 3.00 (m, 1H), 2.43 (m, 2H), 1.85 (m, 3H), 1.61 (m, 1H) 1.50 (m, 1H),
1.27 (m, 1H) and 1.16 (m, 3H).

| | |
|---|---|
| Compound | N-[4-(2-Bromo-3-oxo-3,3a,4,5,6,6a-hexahydro-pentalen-1-yl)-phenyl]-benzamide |
| 146 | |
| R = Benzoyl | |

ES/MS m/z: 398.02 (pos. M + H), 396.01 (neg. M − H); ); ¹H NMR (d6-Acetone, 500 MHz): d 8.06 (m,
2H), 8.04-8.00 (m, 4H), 7.60 (m, 1H), 7.53 (m, 2H), 4.05 (m, 1H), 3.03 (m, 1H), 1.92-1.82 (m, 3H),
1.63 (m, 1H), 1.54 (m, 1H) and 1.30 (m, 1H).

| | |
|---|---|
| Compound | N-[4-(2-Bromo-3-oxo-3,3a,4,5,6,6a-hexahydro-pentalen-1-yl)-phenyl]-butyramide |
| 147 | |
| R = 1-butanoyl | |

ES/MS m/z: 364.01 (pos. M + H), 362.05 (neg. M − H); ¹H NMR (d6-Acetone, 500 MHz): d 7.96 (m,
2H), 7.85 (m, 2H), 4.02 (m, 1H), 3.00 (m, 1H), 2.38 (m, 2H), 1.84 (m, 3H), 1.71 (m, 2H), 1.61 (m, 1H)
1.50 (m, 1H), 1.26 (m, 1H) and 0.96 (m, 3H).

### Compound 148

### (rac)-(3a-8,5R,6aR)-5-Hydroxy-3-(4-hydroxy-phenyl)-5-methyl-4,5,6,6a-tetrahydro-3aH-pentalen-1-one

ES/MS m/z: 245.13 (pos. M+H), 243.09 (neg. M−H); ¹HNMR (CDCl₃/CD₃OD, 500 MHz): d 7.46-7.52 (m, 2H), 6.80-6.85 (m, 2H), 6.28 (d, J=1.2 Hz, 1H), 3.73-3.79 (m, 1H), 2.93-3.01 (m, 1H), 1.98-2.07 (m, 2H), 1.92 (dd, J=13.5, 9.9 Hz, 1H), 1.70-1.76 (m, 1H) and 1.26 (s, 314).

### Binding Assay 1: Estrogen Receptor Binding Assay

The estrogen receptor ligand binding assays are designed as scintillation proximity assays (SPA), employing the use of tritiated estradiol (³H-E2) and recombinant expressed biotinylated estrogen receptor binding domains. The binding domains of human ERα (ERα-LBD, pET-N-AT #1, aa 301-595) and ERβ (ERβ-LBD, pET-N-AT #1, aa 255-530) proteins are produced in E. coli ((BL21, (DE3), pBirA)) at 22° C. in 2×LB medium supplemented with 50 uM biotin. After 3 h of IPTG induction (0.55 mM), cells are harvested by centrifugation at 7300×g for 15 min and cell pellets stored frozen in −20° C. Extraction of ERα and ERβ are performed using 5 g of cells suspended in 50 mL of extraction buffer (50 mM Tris, pH 8.0, 100 mM KCl, 4 mM EDTA, 4 mM DDT and 0.1 mM PMSF). The cell suspension is run twice through a Microfluidizer M-110L (Microfluidics) and centrifuged at 15,000×g for 60 min. The supernatant is aliquoted and stored in −70° C.

Dilute ERα-LBD or ERβ-LBD extracts in assay buffer (18 mM K₂HPO₄, 2 mM KH₂PO₄, 20 mM NaₛMoO₄, 1 mM EDTA, 1 mM TCEP) 1:676 and 1:517 for alpha and beta respectively. The diluted receptor concentrations should be 900 fmol/L. Preincubate the extracts with streptavidin coated polyvinyltoluene SPA beads (RPNQ0007, GE Healthcare) at a concentration of 0.43 mg/mL for 1 hr at room temperature.

Test compounds are evaluated over a range of concentrations from 157 μM to 37.5 μM. The test compound stock solutions should be made in 100% DMSO at 5× of the final concentration desired for testing in the assay. The amount of DMSO in the test wells of the 384 well plate will be 20%. Add 18 μl aliquots of test compounds to the assay plates followed by 35 μl of the preincubated receptor/SPA bead mix and finally add 35 μl of 3 nM ³H-E2. Cover the plates with a plastic sealer, centrifuge for 1 minute at 1000 rpm and equilibrate over night on a shaker at room temperature. The following morning, centrifuge the plates 5 minutes at 2000 rpm and measure on a plate scintillation counter e.g. a PerkinElmer Microbeta 1450 Trilux.

For compounds able to displace 3[H]-E2 from the receptor an IC₅₀-value (the concentration required to inhibit 50% of the binding of 3[H]-E2) is determined by a non-linear four parameter logistic model; b=((borax−bmin)/(1+(I/IC₅₀)S))+bmin I is added concentration of binding inhibitor, IC₅₀ is the concentration of inhibitor at half maximal binding and S is a slope factor. The Microbeta-instrument generates the mean cpm (counts per minute) value/minute and corrects for individual variations between the detectors thus generating corrected cpm values.

### Binding Assay 2: Estrogen Receptor Filter Binding Assay

The ligand binding domain of the human estrogen receptor beta (hERβ-LBD) is used in a competition binding assay with filter separation of bound and free ligand. The assay utilizes tritiated estradiol (³H-E2) as beta particle emitting tracer and recombinant expressed human estrogen beta receptor binding domain. The binding domain of human ERβ (hERβ-LBD, pET-N-AT #1, aa 255-530) protein is produced in Escherichia coli ((BL21, (DE3), pBirA)) at 22° C. in 2×LB medium supplemented with 50 μM biotin. After 3 h of isopropyl 13-D-1-thiogalactopyranoside induction (0.55 mM), cells are harvested by centrifugation at 7300×g for 15 min and cell pellets stored frozen in −20° C. Extraction of hERβ-LBD is performed using 5 g of cells suspended in 50 mL of extraction buffer (50 mM Tris, pH 8.0, 100 mM KCl, 4 mM ethylenediaminetetraacetic acid (EDTA), 4 mM dithiothreitol and 0.1 mM phenylmethanesulfonyl fluoride (TCEP). The cell suspension is run twice through a Microfluidizer M-110L (Microfluidics) and centrifuged at 15,000×g for 60 min. The supernatant is aliquoted and stored in −70° C. Estrogen receptor extract is diluted 1:400 in assay buffer (18 mM K₂HPO₄, 2 mM KH₂PO₄, 20 mM Na₂MoO₄, 1 mM EDTA, 1 mM TCEP, pH 8.0). Test compounds are evaluated over a range of concentrations from 2 μM to 10 μM. The test compound stock solutions should be made in 100% dimethyl sulfoxide (DMSO) at 51× of the final concentration desired for testing in the assay. The final fraction of DMSO in the wells of the 96 well assay plate will be 2%. 100 μl ³H-E2 is added to the assay plates followed by 4 μl aliquots of test compounds and 100 μl of the diluted receptor extract. The assay plates are stored over night at +4° C. Receptor bound and free tracer are separated over a glass fiber filter (FILTERMAT B, PerkinElmer)) on a cell harvester (TOMTECMACH3, Tomtec) with wash buffer (18 mM K₂HPO₄, 2 mM KH₂PO₄, 0.5 mM EDTA). The filters are dried at 60° C. for 1 hour and then merged by heat with a scintillating wax (MELTILEX, PerkinElmer) before measuring on a plate beta counter (Wallac Microbeta Trilux 1450-028, PerkinElmer). The Trilux-instrument generates mean counts per minute (cpm) and corrects for individual variations between the detectors, thus generating corrected cpm values (ccpm). The 1050 values, defined as the midpoint between maximum binding and minimum binding on the sigmoid binding curve, are calculated with XLfit software version 2.0 or later (IDBS) with a four parameter logistic model; b=((bmax−bmin)/(1+(I/IC₅₀)S))+bmin where I is added concentration of binding inhibitor, IC₅₀ is the concentration of inhibitor at half maximal binding and S is a slope factor.

### Binding Assay 3: Estrogen Receptor Time-Resolved Fluorescence Resonance Energy Transfer Competitive Binding Assay

Compounds are tested for their affinity to the ligand binding domain (LBD) of the estrogen receptor beta (ERβ) by concentration-response using a time-resolved fluorescence resonance energy transfer (TR-FRET) competitive binding assay. All materials are provided by Invitrogen (Madison, Wis., USA). Ligands are identified by their ability to compete with and displace a green fluorescent estrogen receptor (ER) ligand, Fluormone™ ES2 (tracer), from the receptor. A purified, glutathione S-transferase (GST)-tagged ER-LBD (ER-LBD-GST) is indirectly labeled using a terbium (Tb)-labeled anti-GST tag antibody. The binding of Fluormone™ ES2 is measured by monitoring fluorescence resonance energy transfer (FRET) from the terbium-labeled antibody to the green tracer, resulting in a high TR-FRET ratio (520 nm fluorescent emission of Fluormone™ ES2: 495 nm fluorescent emission of terbium). Competing ligand will displace Fluormone™ ES2 from the receptor and disrupt FRET, resulting in a lower TR-FRET ratio. Compounds are dissolved in DMSO to a concentration of 10 mM. Dilution series of the compounds are made in 100% DMSO and then further diluted to 4% DMSO in ES2 Screening buffer (Invitrogen P2616), supplemented with 5 mM DTT. Five μl of the compounds is dispensed to a black 384 well assay plate (Corning #3677). A mixture of purified ERβ-LBD-GST (Invitrogen PV4538/37386B) and Tb anti-GST antibody (Invitrogen PV3550/408-416B) is prepared and 5 μl is dispensed to all wells of the assay plate. Ten μl of the tracer, Fluoromonene™ ES2 (Invitrogen P2613/16353B), is dispensed to all wells of the assay plate. The assay plate is shaken on an orbital shaker for 15 seconds and then incubated at room temperature for two hours, protected from light and evaporation. Final concentrations in the assay plate are; 0.5 nM ERβ-LBD-GST, 2 nM Tb anti-GST antibody, 3 nM Fluoromonem ES2 tracer, 1× screening buffer, 5 mM DTT, 1% DMSO, 100 μM to 7 pM test compound distributed over 16 concentrations. The 520/495 TR-FRET ratio is measured using a Tecan Infinite 500 instrument with excitation filter 340 nm (30 nm bandwidth) and emission filters 495 nm (10 nm bandwidth) and 520 nm (25 nm bandwidth). A 200 μs integration time follows a 100 μs delay to collect the time-resolved signal.

The IC₅₀ values, defined as the midpoint between maximum ratio and minimum ratio on the sigmoid binding curve, are calculated with XLfit software version 2.0 or later (IDBS) with a four parameter logistic model; b=((bmax−bmin)/(1+(I/IC₅₀)S))+bmin where I is added concentration of competing ligand, IC₅₀ is the concentration of competing ligand at the midpoint between maximum ratio (bmax) and minimum ratio (bmin) and S is a slope factor.

Transactivation Assay 1: Transactivation Assay in Human Embryonic Kidney 293 Cells Stably Transfected with pERE-ALP and Human Estrogen Receptor Alpha

The expression vector pMThERα contains an insert of wild type human estrogen receptor alpha with deleted leader. The pERE-ALP reporter construct contains the gene for the secreted form of placental alkaline phosphatase (ALP) and the vitellogenin estrogen response element (ERE). The human embryonic kidney 293 cells are transfected in two steps. Firstly, a stable clone mix transfected with the pERE-ALP reporter gene construct and pSV2-Neo for selection is developed. Secondly, the stable clone mix is transfected with pMThERα and a pKSV-Hyg resistance vector for selection. All transfections are performed using Lipofectamine (Invitrogen) according to supplier's recommendations. A selected clone with both pERE-ALP and pMThERα is used for the transactivation assay.

The cells are seeded in 384-well plates at 12 500 cells per well in Ham's F12 Coon's modification (without phenol red) with 10% dextran-coated charcoal treated (DCC) fetal bovine serum (FBS), 2 mM L-glutamine and 50 μg/ml gentamicin. After 24 h incubation (37° C., 5% CO₂) the seeding medium is discarded and replaced with 20 μl Ham's F12 Coon's modification (without phenol red) with 1.5% DCC-FCS, 2 mM L-glutamine and supplemented with 100 U/ml penicillin and 100 μg/ml streptomycin. The selected compounds are added to the wells in 12 concentrations ranging from 3.3 μM to 33 μM. The compounds are dissolved in 100% dimethylsulphoxide (DMSO) and the final concentration of DMSO in the assay is 0.1%. After 72 h incubation (37° C., 5% CO₂) the medium is assayed for ALP activity by a chemiluminescence assay; a 10 μl aliquot of the cell culture medium is mixed with 100 μl assay buffer (0.1 M diethanolamine, 1 mM MgCl₂) and 0.5 mM disodium 3-(4-methoxyspiro 1,2-dioxetane-3,2′-(5′-chloro)-tricyclo[3.3.1.13,7]decan-4-yl)phenyl phosphate (CSPD) (Tropix, Applied Biosystems) and incubated for 20 min at 37° C. and 15 min at room temperature before measurement chemiluminescent light signal (one second per well) in a Wallac Microbeta Trilux 1450-028 (PerkinElmer). The half maximal effective concentrations (EC₅₀) are calculated from the curves fitted to the concentration-response data with a four parameter logistic model in XLfit software version 2.0 (IDBS) or later.

Transactivation Assay 2: Transactivation Assay in Human Embryonic Kidney 293 Cells Stably Transfected with pERE-ALP and Transiently Transfected with Rat Estrogen Receptor Alpha or Beta

The pERE-ALP reporter construct contains the gene for the secreted form of placental alkaline phosphatase (ALP) and the vitellogenin estrogen response element (ERE).

The human embryonic kidney 293 cells are stably transfected with the pERE-ALP reporter gene construct and pSV2-Neo for selection using Lipofectamine according to supplier's recommendations.

The cells are seeded at 25 000 cells per well in 384-well plates. When seeded, the cells are transfected with 31 ng full length rat estrogen receptor alpha or beta per well using Lipofectamine (Invitrogen) according to supplier's recommendations. After 20 h incubation (37° C., 5% CO₂) the transfection medium is discarded and replaced with 20 μl Ham's F12 Coon's modification (without phenol red) with 1.5% DCC-FCS, 2 mM L-glutamine and supplemented with 100 U/ml penicillin and 100 μg/ml streptomycin. The selected compounds are added to the wells in 12 concentrations ranging from 3.3 μM to 33 μM. The compounds are dissolved in 100% dimethylsulphoxide (DMSO) and the final concentration of DMSO in the assay is 0.1%. After 72 h incubation (37° C., 5% CO₂) the medium is assayed for ALP activity by a chemiluminescence assay; a 10 μl aliquot of the cell culture medium is mixed with 100 μl assay buffer (0.1 M diethanolamine, 1 mM MgCl₂) and 0.5 mM disodium 3-(4-methoxyspiro-1,2-dioxetane-3,2′-(5′-chloro)-tricyclo[3.3.1.13,7]decan-4-yl)phenyl phosphate (CSPD) (Tropix, Applied Biosystems) and incubated for 20 min at 37° C. and 15 min at room temperature before measurement chemiluminescent light signal (one second per well) in a Wallac Microbeta Trilux 1450-028 (PerkinElmer). The half maximal effective concentrations (EC₅₀) are calculated from the curves fitted to the concentration-response data with a four parameter logistic model in XLfit software version 2.0 (IDBS) or later.

The compounds of Examples 1-132 exhibit one or more of the following:

(i) a binding affinity to the estrogen receptor α-subtype in the range of IC₅₀ 1 to 10,000 nM or to the estrogen receptor β-subtype in the range of IC₅₀ 1 to 10,000 nM in binding assay 1;
(ii) a binding affinity to the estrogen receptor α-subtype in the range of IC₅₀ 1 to 10,000 nM or to the estrogen receptor β-subtype in the range of IC₅₀ 1 to 10,000 nM in binding assay 2;
(iii) a binding affinity to the estrogen receptor α-subtype in the range of IC₅₀ 1 to 10,000 nM or to the estrogen receptor β-subtype in the range of IC₅₀ 1 to 10,000 nM in binding assay 3;
(iv) a potency in the range of EC₅₀ 1 to 10,000 nM at the estrogen receptor α-subtype in transactivation assay 1;
(v) a potency in the range of EC₅₀ 1 to 10,000 nM at the estrogen receptor α-subtype or a potency in the range of EC₅₀ 1 to 10,000 nM at the estrogen receptor β-subtype in transactivation assay 2.

1. A compound of formula (I) or a pharmaceutically acceptable ester, amide, solvate or salt thereof, including a salt of such an ester or amide, and a solvate of such an ester, amide or salt, 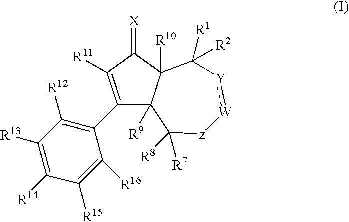
wherein
Y is selected from a bond, CR³R³⁰, C═CR³R³⁰ and NR³¹;
W is selected from a bond, CR⁴R⁴⁰ and C═CR⁴R⁴⁰; NR⁴¹;
and when both Y and W are not bond, then the bond between Y and W is a single bond or a double bond, and when it is a double bond Y is CR³ and W is CR⁴;
Z is selected from a bond, CR⁵R⁶ and C═CR⁵R⁶;
R¹, R², R³, R⁴, R⁵R⁶, R⁷, R⁸, R³⁰ and R⁴⁰ are the same or are different and each is selected from the group consisting of hydrogen, OR^{D}, halogen, amino, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl, C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R³¹ and R⁴¹ are the same or are different and each is selected from the group consisting of hydrogen, OR^{A}, C₁₋₆ alkyl, C₂₋₆ alkenyl, C₂₋₆ alkynyl, halo C₁₋₆ alkyl, dihalo C₁₋₆ alkyl, trihalo C₁₋₆ alkyl, C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
each R^{A} is independently selected from the group consisting of hydrogen, C₁₋₆ alkyl, C₂₋₆ alkenyl, C₂₋₆ alkynyl, C₃₋₈ cycloalkyl, C₃₋₈ cycloalkyl C₁₋₆ alkyl, phenyl, benzyl and C₅₋₈ heterocyclyl, each of said alkyl, alkenyl and alkynyl groups or parts of groups being optionally substituted with 1-3 substituents and each substituent is independently selected from the group consisting of OR^{A}, halogen, cyano and nitro; each of said cycloalkyl, phenyl, benzyl or C₅₋₈ heterocyclyl groups or parts of groups being optionally substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
each R^{D} is independently selected from the group consisting of C₁₋₆ alkyl, C₂₋₆ alkenyl, C₂₋₆ alkynyl, C₃₋₈ cycloalkyl, C₃₋₈ cycloalkyl C₁₋₆ alkyl, phenyl, benzyl and C₅₋₈ heterocyclyl, each of said alkyl, alkenyl and alkynyl groups or parts of groups being optionally substituted with 1-3 substituents and each substituent is independently selected from the group consisting of OR^{A}, halogen, cyano and nitro; each of said cycloalkyl, phenyl, benzyl or C₅₋₈ heterocyclyl groups or parts of groups being optionally substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R⁹ and R¹⁰ are the same or different and each is selected from the group consisting of hydrogen, halogen, OR^{A}, C₁₋₆ alkyl, halo C₁₋₆ alkyl, dihalo C₁₋₆ alkyl and trihalo C₁₋₆ alkyl;
X is selected from O and NOR^{E};
R^{E} is selected from the group consisting of hydrogen, C₁₋₆ alkyl and phenyl;
R¹¹ is selected from the group consisting of hydrogen, halogen, cyano, OR^{A}, —C(O)C₁₋₄ alkyl, C₁₋₆ alkyl, halo C₁₋₆ alkyl, dihalo C₁₋₆ alkyl, trihalo C₁₋₆alkyl, C₂₋₆ alkenyl, C₂₋₆ alkynyl, C₃₋₈ cycloalkyl, C₃₋₈ cycloalkyl C₁₋₆ alkyl, phenyl, benzyl and C₅₋₁₀ heterocyclyl wherein said phenyl, benzyl or C₅₋₁₀ heterocyclyl group can either be unsubstituted or substituted with 1-3 substituents and each substituent is independently selected from the group consisting of OR^{A}, halogen, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R¹² and R¹⁶ are the same or are different and each is selected from the group consisting of hydrogen, OR^{A}, halogen, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R¹³ and R¹⁵ are the same or different and each is selected from the group consisting of hydrogen, halogen, nitro, OR^{A}, N(R^{B})₂, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl;
R¹⁴ is selected from the group consisting of, OR^{A}, N(R^{C})₂, —C(O)C₁₋₄alkyl, —C(O)phenyl, and —O—C(O)R^{A}; or R¹⁴ and R¹⁵ or R¹³ and R¹⁴ may, together with the atoms they are attached to, form a 5-, 6- or 7-membered cyclic group optionally containing one to three heteroatoms selected from O, N and S, said 5-, 6- or 7-membered cyclic group being optionally substituted with one of more groups selected from OR^{A}, cyano, nitro, C₁₋₆ alkyl, C₂₋₆ alkenyl, C₂₋₆ alkynyl, halo C₁₋₆ alkyl, dihalo C₁₋₆ alkyl and trihalo C₁₋₆ alkyl; and
each R^{B} is independently selected from the group consisting of hydrogen, —C(O)C₁₋₄ alkyl, —C(O)phenyl, —SO₂C₁₋₄ alkyl, —SO₂-phenyl, C₁₋₆ alkyl, C₂₋₆ alkenyl, C₂₋₆ alkynyl, C₃₋₈ cycloalkyl, C₃₋₈ cycloalkyl C₁₋₆ alkyl, phenyl, benzyl, C₅₋₁₀ heterocyclyl and C₅₋₁₀ heterocyclyl C₁₋₆alkyl; and
each R^{C} is independently selected from the group consisting of hydrogen, —C(O)Me, C₁₋₆ alkyl, C₂₋₆ alkynyl, C₃₋₈ cycloalkyl, C₃₋₈ cycloalkyl C₁₋₆ alkyl, phenyl, benzyl, C₅₋₁₀heterocyclyl and C₅₋₁₀ heterocyclyl C₁₋₆ alkyl.

2. A compound as claimed in claim 1 wherein
Y is selected from a bond, CR³R³⁰ and C═CR³R³⁰;
W is selected from a bond, CR⁴K and C═CR⁴R⁴⁰;
and when both Y and W are not bond, then the bond between Y and W is a single bond or a double bond, and when it is a double bond Y is CR³ and W is CR⁴;
Z is selected from a bond or CR⁵R⁶;
R¹, R², R³, R⁴, R⁵, R⁶, R⁷, R⁸, R³⁰ and R⁴⁰ are the same or are different and each is selected from the group consisting of hydrogen, OR^{D}, halogen, C₁₋₄ alkyl, halo C₁₋₄ alkyl, dihalo C₁₋₄ alkyl and trihalo C₁₋₄ alkyl;
each R^{A} is independently selected from the group consisting of hydrogen, C₁₋₄ alkyl, C₃₋₆ cycloalkyl, phenyl and benzyl;
each R^{D} is independently selected from the group consisting of C₁₋₄ alkyl, C₃₋₆ cycloalkyl, phenyl and benzyl;
R⁹ and R¹⁰ are the same or different and each is selected from the group consisting of hydrogen, halogen, OR^{A}, C₁₋₄ alkyl, halo C₁₋₄ alkyl, dihalo C₁₋₄ alkyl and trihalo C₁₋₄ alkyl;
X is selected from O and NOH;
R¹¹ is selected from the group consisting of hydrogen, halogen, cyano, —C(O)C₁₋₄ alkyl, C₁₋₄ alkyl, halo C₁₋₄ alkyl, dihalo C₁₋₄ alkyl, trihalo C₁₋₄ alkyl, C₂₋₆ alkenyl, C₂₋₄ alkynyl, C₃₋₆ cycloalkyl, C₃₋₆ cycloalkyl C₁₋₂ alkyl, phenyl, benzyl and C₅₋₆ heterocyclyl wherein said phenyl, benzyl or C₅₋₆ heterocyclyl group can either be unsubstituted or substituted with 1-3 substituents and each substituent is independently selected from the group consisting of OR^{A}, halogen, cyano, nitro, C₁₋₂ alkyl, halo C₁₋₂ alkyl, dihalo C₁₋₂alkyl and trihalo C₁₋₂alkyl;
R¹² and R¹⁶ are the same or are different and each is selected from the group consisting of hydrogen, OR^{A}, halogen, C₁₋₄ alkyl, halo C₁₋₄ alkyl, dihalo C₁₋₄ alkyl and trihalo C₁₋₄ alkyl;
R¹³ and R¹⁵ are the same or different and each is selected from the group consisting of hydrogen, halogen, OR^{A}, N(R^{B})₂, C₁₋₄ alkyl, halo C₁₋₄alkyl, dihalo C₁₋₄ alkyl and trihalo C₁₋₄ alkyl;
R¹⁴ is selected from the group consisting of hydrogen, OR^{A}, N(R^{C})₂, —C(O)C₁₋₄ alkyl, —C(O)phenyl, and —O—C(O)R^{A} or R¹⁴ and R¹⁵ or R¹³ and R¹⁴ may, together with the atoms they are attached to, form a 5-, 6- or 7-membered cyclic group optionally containing one to three heteroatoms selected from O and N; and
each R^{B} is independently selected from the group consisting of hydrogen, —C(O)C₁₋₄ alkyl, and C₁₋₄ alkyl; and
each R^{C} is independently selected from the group consisting of hydrogen, —C(O)Me, and C₁₋₄ alkyl.

3. A compound as claimed in claim 1 wherein
Y is selected from a bond or CR³R³⁰;
W is selected from a bond or CR⁴R⁴⁰;
and when both Y and W are not bond, then the bond between Y and W is a single bond or a double bond, and when it is a double bond Y is CR³ and W is CR⁴;
Z is selected from a bond or CR⁵R⁶;
R¹, R², R³, R⁴, R⁵, R⁶, R⁷, R⁸, R³⁰ and R⁴⁰ are the same or are different and each is selected from the group consisting of hydrogen, OR^{D}, halogen, C₁₋₄ alkyl, halo C₁₋₄ alkyl, dihalo C₁₋₄ alkyl and trihalo C₁₋₄ alkyl;
each R^{D} is independently selected from the group consisting of C₁₋₄ alkyl, C₃₋₆ cycloalkyl, phenyl and benzyl;
each R^{A} is independently selected from the group consisting of hydrogen, C₁₋₄ alkyl, C₃₋₆ cycloalkyl, phenyl and benzyl;
R⁹ and R¹⁰ are the same or different and each is selected from the group consisting of hydrogen, halogen, OR^{A}, C₁₋₄ alkyl, halo C₁₋₄ alkyl, dihalo C₁₋₄ alkyl and trihalo C₁₋₄ alkyl;
X is selected from O and NOH;
R¹¹ is selected from the group consisting of hydrogen, halogen, cyano, —C(O)C₁₋₄ alkyl, C₁₋₄ alkyl, halo C₁₋₄ alkyl, dihalo C₁₋₄ alkyl, trihalo C₁₋₄ alkyl, C₂₋₆ alkenyl, C₂₋₄ alkynyl, C₃₋₆ cycloalkyl, C₃₋₆ cycloalkyl C₁₋₂ alkyl, phenyl, benzyl and C₅₋₆ heterocyclyl wherein said phenyl, benzyl or C₅₋₆ heterocyclyl group can either be unsubstituted or substituted with 1-3 substituents and each substituent is independently selected from the group consisting of OR^{A}, halogen, cyano, nitro, C₁₋₂ alkyl, halo C₁₋₂ alkyl, dihalo C₁₋₂ alkyl and trihalo C₁₋₂ alkyl;
R¹² and R¹⁶ are the same or are different and each is selected from the group consisting of hydrogen, OR^{A}, halogen, C₁₋₄ alkyl, halo C₁₋₄ alkyl, dihalo C₁₋₄ alkyl and trihalo C₁₋₄ alkyl;
R¹³ and R¹⁵ are the same or different and each is selected from the group consisting of hydrogen, halogen, OR^{A}, N(R^{B})₂, C₁₋₄ alkyl, halo C₁₋₄ alkyl, dihalo C₁₋₄ alkyl and trihalo C₁₋₄ alkyl;
R¹⁴ is selected from the group consisting of hydrogen, OR^{A}, N(R^{C})₂, —C(O)C₁₋₄ alkyl, —C(O)phenyl, and —O—C(O)R^{A} or R¹⁴ and R¹⁵ or R¹³ and R¹⁴ may, together with the atoms they are attached to, form a 5-, 6- or 7-membered cyclic group optionally containing one to three heteroatoms selected from O and N;
each R^{B} is independently selected from the group consisting of hydrogen, —C(O)C₁₋₄ alkyl, and C₁₋₄ alkyl; and
each R^{C} is independently selected from the group consisting of hydrogen, —C(O)Me, and C₁₋₄ alkyl.

4. A compound as claimed in claim 1 which is a compound of formula (Ia) or a pharmaceutically acceptable ester, amide, solvate or salt thereof, including a salt of such an ester or amide, and a solvate of such an ester, amide or salt: 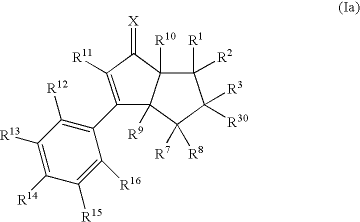
wherein
R¹, R², R³, R⁷, R⁸ and R³⁰ are the same or are different and each is selected from the group consisting of hydrogen, halogen, C₁₋₄ alkyl, halo C₁₋₄ alkyl, dihalo C₁₋₄ alkyl and trihalo C₁₋₄ alkyl;
each R^{A} is independently selected from the group consisting of hydrogen, C₁₋₄ alkyl, C₃₋₆ cycloalkyl, phenyl and benzyl;
R⁹ and R¹⁰ are the same or different and each is selected from the group consisting of hydrogen, halogen, C₁₋₄ alkyl, halo C₁₋₄ alkyl, dihalo C₁₋₄ alkyl and trihalo C₁₋₄ alkyl;
X is selected from O and NOH;
R¹¹ is selected from the group consisting of hydrogen, halogen, cyano, —C(O)C₁₋₄ alkyl, C₁₋₄ alkyl, halo C₁₋₄ alkyl, dihalo C₁₋₄ alkyl, trihalo C₁₋₄ alkyl, C₂₋₆ alkenyl, C₂₋₄ alkynyl, C₃₋₆ cycloalkyl, C₃₋₆ cycloalkyl C₁₋₂ alkyl, phenyl, benzyl and C₅₋₆ heterocyclyl wherein said phenyl, benzyl or C₅₋₆ heterocyclyl group can either be unsubstituted or substituted with 1-3 substituents and each substituent is independently selected from the group consisting of OR^{A}, halogen, cyano, nitro, C₁₋₂ alkyl, halo C₁₋₂ alkyl, dihalo C₁₋₂ alkyl and trihalo C₁₋₂ alkyl;
R¹² and R¹⁶ are the same or are different and each is selected from the group consisting of hydrogen, halogen, C₁₋₄ alkyl, halo C₁₋₄ alkyl, dihalo C₁₋₄alkyl and trihalo C₁₋₄ alkyl;
R¹³ and R¹⁵ are the same or different and each is selected from the group consisting of hydrogen, halogen, C₁₋₄ alkyl, halo C₁₋₄alkyl, dihalo C₁₋₄ alkyl and trihalo C₁₋₄ alkyl;
R¹⁴ is selected from the group consisting of OR^{A}, N(R^{C})₂, and —O—C(O)R^{A}; and
each R^{C} is independently selected from the group consisting of hydrogen, —C(O)Me, and C₁₋₄ alkyl.

5. A compound as claimed in claim 1 which is selected from:
3-(4-hydroxy-phenyl)-2-phenyl-4,5,6,6a-tetrahydro-3aH-pentalen-1-one (E1);
2-bromo-3-(4-hydroxy-phenyl)-4,5,6,6a-tetrahydro-3aH-pentalen-1-one (E2);
(3aR,6aS)-2-bromo-3-(4-hydroxy-phenyl)-4,5,6,6a-tetrahydro-3aH-pentalen-1-one (E3);
(3a-8,6aR)-2-bromo-3-(4-hydroxy-phenyl)-4,5,6,6a-tetrahydro-3aH-pentalen-1-one (E4);
2-bromo-3-(4-hydroxy-phenyl)-5-methyl-4,5,6,6a-tetrahydro-3aH-pentalen-1-one (E5);
2-bromo-5-ethyl-3-(4-hydroxy-phenyl)-4,5,6,6a-tetrahydro-3aH-pentalen-1-one (E6);
2-chloro-3-(4-hydroxy-phenyl)-4,5,6,6a-tetrahydro-3aH-pentalen-1-one (E7);
3-(4-hydroxy-phenyl)-1-oxo-1,3a,4,5,6,6a-hexahydro-pentalene-2-carbonitrile (E8);
3-(4-hydroxy-phenyl)-2-trifluoromethyl-4,5,6,6a-tetrahydro-3aH-pentalen-1-one (E9);
2-cyclopropyl-3-(4-hydroxy-phenyl)-4,5,6,6a-tetrahydro-3aH-pentalen-1-one (E10);
2,2-dimethyl-propionic acid 4-(2-bromo-3-oxo-3,3a,4,5,6,6a-hexahydro-pentalen-1-yl)-phenyl ester (E11);
2-bromo-6a-fluoro-3-(4-hydroxy-phenyl)-4,5,6,6a-tetrahydro-3aH-pentalen-1-one (E12);
2-bromo-3-(4-hydroxy-phenyl)-6a-methyl-4,5,6,6a-tetrahydro-3aH-pentalen-1-one (E13);
3-(4-hydroxy-phenyl)-3a,4,7,7a-tetrahydro-inden-1-one (E14);
3-(4-hydroxy-phenyl)-3a,4,5,6,7,7a-hexahydro-inden-1-one (E15);
2-bromo-3-(4-hydroxy-phenyl)-3a,4,5,6,7,7a-hexahydro-inden-1-one (E16);
2-bromo-3-(4-hydroxy-phenyl)-4,5,6,6a-tetrahydro-3aH-pentalen-1-one oxime (E17);
N-[4-(2-bromo-3-oxo-3,3a,4,5,6,6a-hexahydro-pentalen-1-yl)-phenyl]-acetamide (E 18);
3-(4-hydroxy-phenyl)-4,5,6,6a-tetrahydro-3aH-pentalen-1-one (E19);
2-bromo-3-(3-bromo-4-hydroxy-phenyl)-4,5,6,6a-tetrahydro-3aH-pentalen-1-one (E20);
2-bromo-3-(3-chloro-4-hydroxy-phenyl)-4,5,6,6a-tetrahydro-3aH-pentalen-1-one (E21);
2-bromo-3-(3,5-dichloro-4-hydroxy-phenyl)-4,5,6,6a-tetrahydro-3aH-pentalen-1-one (E22);
2-bromo-3-(3-fluoro-4-hydroxy-phenyl)-4,5,6,6a-tetrahydro-3aH-pentalen-1-one (E23);
3-(4-hydroxy-3-methyl-phenyl)-4,5,6,6a-tetrahydro-3aH-pentalen-1-one (E24);
3-(2-fluoro-4-hydroxy-phenyl)-4,5,6,6a-tetrahydro-3aH-pentalen-1-one (E25);
2-bromo-3-(4-hydroxy-3-methyl-phenyl)-4,5,6,6a-tetrahydro-3aH-pentalen-1-one (E26);
2-bromo-3-(2-fluoro-4-hydroxy-phenyl)-4,5,6,6a-tetrahydro-3aH-pentalen-1-one (E27);
3-(3-fluoro-4-hydroxy-phenyl)-4,5,6,6a-tetrahydro-3aH-pentalen-1-one (E28);
2-bromo-3-(3-chloro-5-fluoro-4-hydroxy-phenyl)-4,5,6,6a-tetrahydro-3aH-pentalen-1-one (E29);
2-chloro-3-(3-chloro-5-fluoro-4-hydroxy-phenyl)-4,5,6,6a-tetrahydro-3aH-pentalen-1-one (E30);
3-(4-hydroxy-phenyl)-2-thiophen-2-yl-4,5,6,6a-tetrahydro-3aH-pentalen-1-one (E31);
3-(4-hydroxy-phenyl)-2-(3-methyl-thiophen-2-yl)-4,5,6,6a-tetrahydro-3aH-pentalen-1-one (E32);
3-(4-hydroxy-phenyl)-2-prop-1-ynyl-4,5,6,6a-tetrahydro-3aH-pentalen-1-one (E33);
2-ethynyl-3-(4-hydroxy-phenyl)-4,5,6,6a-tetrahydro-3aH-pentalen-1-one (E34);
2-[3-(4-hydroxy-phenyl)-1-oxo-1,3a,4,5,6,6a-hexahydro-pentalen-2-yl]-thiophene-3-carbonitrile (E35);
2-furan-2-yl-3-(4-hydroxy-phenyl)-4,5,6,6a-tetrahydro-3aH-pentalen-1-one (E36);
3-(4-hydroxy-phenyl)-2-vinyl-4,5,6,6a-tetrahydro-3aH-pentalen-1-one (E37);
3-(4-hydroxy-phenyl)-2-(2-methoxy-thiazol-4-yl)-4,5,6,6a-tetrahydro-3aH-pentalen-1-one (E38);
3-(4-hydroxy-phenyl)-2-thiazol-4-yl-4,5,6,6a-tetrahydro-3aH-pentalen-1-one (E39);
3-(4-hydroxy-phenyl)-2-thiazol-2-yl-4,5,6,6a-tetrahydro-3aH-pentalen-1-one (E40);
3-(4-hydroxy-phenyl)-2-(2-methyl-allyl)-4,5,6,6a-tetrahydro-3aH-pentalen-1-one (E41);
3-(4-hydroxy-phenyl)-2-((E)-propenyl)-4,5,6,6a-tetrahydro-3aH-pentalen-1-one (E42);
3-(4-hydroxy-phenyl)-2-((Z)-propenyl)-4,5,6,6a-tetrahydro-3aH-pentalen-1-one (E43);
3-(4-hydroxy-phenyl)-2-(3-methyl-but-2-enyl)-4,5,6,6a-tetrahydro-3aH-pentalen-1-one (E44);
2-acetyl-3-(4-hydroxy-phenyl)-4,5,6,6a-tetrahydro-3aH-pentalen-1-one (E45);
3-(4-hydroxy-phenyl)-2-thiophen-3-yl-4,5,6,6a-tetrahydro-3aH-pentalen-1-one (E46);
3-(4-hydroxy-phenyl)-2-isopropenyl-4,5,6,6a-tetrahydro-3aH-pentalen-1-one (E47);
3-(4-hydroxy-phenyl)-2-(1-methyl-1H-pyrrol-2-yl)-4,5,6,6a-tetrahydro-3aH-pentalen-1-one (E48);
benzoic acid 4-(2-bromo-3-oxo-3,3a,4,5,6,6a-hexahydro-pentalen-1-yl)-phenyl ester (E49);
2-bromo-3-(4-dimethylamino-phenyl)-4,5,6,6a-tetrahydro-3aH-pentalen-1-one (E50);
2-bromo-3-(4-hydroxy-2,5-dimethyl-phenyl)-4,5,6,6a-tetrahydro-3aH-pentalen-1-one (E51);
3-(6-hydroxy-naphthalen-2-yl)-4,5,6,6a-tetrahydro-3aH-pentalen-1-one (E52);
2-bromo-3-(4-hydroxy-3,5-dimethyl-phenyl)-4,5,6,6a-tetrahydro-3aH-pentalen-1-one (E53);
2-bromo-3-(4-hydroxy-2-methyl-phenyl)-4,5,6,6a-tetrahydro-3aH-pentalen-1-one (E54); 3a-bromo-3-(3,5-difluoro-4-hydroxy-phenyl)-4,5,6,6a-tetrahydro-3aH-pentalen-1-one (E55);
2-(3,5-dimethyl-isoxazol-4-yl)-3-(4-hydroxy-phenyl)-4,5,6,6a-tetrahydro-3aH-pentalen-1-one (E56);
3-(4-amino-3-methyl-phenyl)-2-bromo-4,5,6,6a-tetrahydro-3aH-pentalen-1-one (E57);
3-(4-amino-phenyl)-4,5,6,6a-tetrahydro-3aH-pentalen-1-one (E58);
3-(4-amino-phenyl)-2-bromo-4,5,6,6a-tetrahydro-3aH-pentalen-1-one (E59);
3-(4-amino-3-bromo-phenyl)-2-bromo-4,5,6,6a-tetrahydro-3aH-pentalen-1-one (E60);
2-bromo-3-(1H-indazol-5-yl)-4,5,6,6a-tetrahydro-3aH-pentalen-1-one (E61);
3-(1H-indazol-5-yl)-1-oxo-1,3a,4,5,6,6a-hexahydro-pentalene-2-carbonitrile (E62);
3-(1H-indazol-5-yl)-4,5,6,6a-tetrahydro-3aH-pentalen-1-one (E63);
2-[3-(1H-indazol-5-yl)-1-oxo-1,3a,4,5,6,6a-hexahydro-pentalen-2-yl]-thiophene-3-carbonitrile (E64);
2-bromo-3-(4-isobutylamino-phenyl)-4,5,6,6a-tetrahydro-3aH-pentalen-1-one (E 65);
2-Bromo-3-(4-methylamino-phenyl)-4,5,6,6a-tetrahydro-3aH-pentalen-1-one (E66);
2-bromo-3-{4-[(furan-2-ylmethyl)-amino]-phenyl}-4,5,6,6a-tetrahydro-3aH-pentalen-1-one (E67);
2-Bromo-3-(4-pentylamino-phenyl)-4,5,6,6a-tetrahydro-30-1-pentalen-1-one (E68);
2-bromo-3-(4-hydroxy-phenyl)-5-methylene-4,5,6,6a-tetrahydro-3aH-pentalen-1-one (E69);
3-(4-hydroxy-phenyl)-5-methylene-4,5,6,6a-tetrahydro-3aH-pentalen-1-one (E 70);
2-benzyl-6-(4-hydroxy-phenyl)-2,3,3a,6a-tetrahydro-1H-cyclopenta[c]pyrrol-4-one (E71);
(rac)-(3aS,5R,6aR)-5-bromo-3-(4-hydroxy-phenyl)-5-methyl-4,5,6,6a-tetrahydro-3aH-pentalen-1-one (E72);
(rac)-(3aS,5R,6aR)-2,5-dibromo-3-(4-hydroxy-phenyl)-5-methyl-4,5,6,6a-tetrahydro-3aH-pentalen-1-one (E73);
(rac)-(3aS,5S,6aR)-2,5-dibromo-3-(4-hydroxy-phenyl)-5-methyl-4,5,6,6a-tetrahydro-3aH-pentalen-1-one (E74);
(rac)-(3aS,5S,6aR)-5-chloro-3-(4-hydroxy-phenyl)-5-methyl-4,5,6,6a-tetrahydro-3aH-pentalen-1-one (E75);
(rac)-(3aS,5S,6aR)-2-bromo-5-chloro-3-(4-hydroxy-phenyl)-5-methyl-4,5,6,6a-tetrahydro-3aH-pentalen-1-one (E76);
(rac)-(3aS,5R,6aR)-2-Bromo-5-chloro-3-(4-hydroxy-phenyl)-5-methyl-4,5,6,6a-tetrahydro-3aH-pentalen-1-one (E77);
(rac)-(5R,6aS)-2,3a-dibromo-5-chloro-3-(4-hydroxy-phenyl)-5-methyl-4,5,6,6a-tetrahydro-3aH-pentalen-1-one (E78);
(rac)-(5S,6aS)-3a-bromo-5-chloro-3-(4-hydroxy-phenyl)-5-methyl-4,5,6,6a-tetrahydro-3aH-pentalen-1-one (E79);
2-bromo-3-(2,3-difluoro-4-hydroxy-phenyl)-4,5,6,6a-tetrahydro-3aH-pentalen-1-one (E80);
3-(2,3-difluoro-4-hydroxy-phenyl)-1-oxo-1,3a,4,5,6,6a-hexahydro-pentalene-2-carbonitrile (E81);
2-bromo-3-(2,5-difluoro-4-hydroxy-phenyl)-4,5,6,6a-tetrahydro-3aH-pentalen-1-one (E82);
3-(3-fluoro-4-hydroxy-phenyl)-1-oxo-1,3a,4,5,6,6a-hexahydro-pentalene-2-carbonitrile (E83);
2-bromo-3-(3-fluoro-4-hydroxy-phenyl)-6a-methyl-4,5,6,6a-tetrahydro-3aH-pentalen-1-one (E84);
3-(3-fluoro-4-hydroxy-phenyl)-6a-methyl-1-oxo-1,3a,4,5,6,6a-hexahydro-pentalene-2-carbonitrile (E85);
2-bromo-3-(2,3-difluoro-4-hydroxy-phenyl)-6a-methyl-4,5,6,6a-tetrahydro-3aH-pentalen-1-one (E86);
2-bromo-3-(3,5-difluoro-4-hydroxy-phenyl)-4,5,6,6a-tetrahydro-3aH-pentalen-1-one (E87);
3-(2,3-difluoro-4-hydroxy-phenyl)-6a-methyl-1-oxo-1,3a,4,5,6,6a-hexahydro-pentalene-2-carbonitrile (E88);
3-(3-chloro-5-fluoro-4-hydroxy-phenyl)-1-oxo-1,3a,4,5,6,6a-hexahydro-pentalene-2-carbonitrile (E89);
3-(3,5-difluoro-4-hydroxy-phenyl)-3α-hydroxy-1-oxo-1,3a,4,5,6,6a-hexahydro-pentalene-2-carbonitrile (E90);
3-(3,5-difluoro-4-hydroxy-phenyl)-4,5,6,6a-tetrahydro-3aH-pentalen-1-one (E91);
3-(3,5-difluoro-4-hydroxy-phenyl)-1-oxo-1,3a,4,5,6,6a-hexahydro-pentalene-2-carbonitrile (E92);
3-(2,5-difluoro-4-hydroxy-phenyl)-1-oxo-1,3a,4,5,6,6a-hexahydro-pentalene-2-carbonitrile (E93);
2-bromo-3-(2,5-difluoro-4-hydroxy-phenyl)-6a-methyl-4,5,6,6a-tetrahydro-3aH-pentalen-1-one (E94);
3-(2,5-difluoro-4-hydroxy-phenyl)-6a-methyl-1-oxo-1,3a,4,5,6,6a-hexahydro-pentalene-2-carbonitrile (E95); 2,6a-dibromo-3-(2,3-difluoro-4-hydroxy-phenyl)-4,5,6,6a-tetrahydro-3aH-pentalen-1-one (E96);
2-bromo-3-(3,5-difluoro-4-hydroxy-phenyl)-4,5,6,6a-tetrahydro-3aH-pentalen-1-one oxime (E97);
3-(3,5-Difluoro-4-hydroxy-phenyl)-1-hydroxyimino-1,3a,4,5,6,6a-hexahydro-pentalene-2-carbonitrile (E98);
2-bromo-3-(3,5-difluoro-4-hydroxy-phenyl)-6a-methyl-4,5,6,6a-tetrahydro-3aH-pentalen-1-one (E99);
3-(3,5-difluoro-4-hydroxy-phenyl)-6a-methyl-1-oxo-1,3a,4,5,6,6a-hexahydro-pentalene-2-carbonitrile (E100);
2-bromo-3-(3,5-difluoro-4-hydroxy-phenyl)-6a-methyl-4,5,6,6a-tetrahydro-3aH-pentalen-1-one oxime (E101);
3-(3-chloro-4-hydroxy-phenyl)-1-oxo-1,3a,4,5,6,6a-hexahydro-pentalene-2-carbonitrile (E102);
2-bromo-3-(3-chloro-5-fluoro-4-hydroxy-phenyl)-6a-methyl-4,5,6,6a-tetrahydro-3aH-pentalen-1-one (E103);
3-(3-chloro-5-fluoro-4-hydroxy-phenyl)-6a-methyl-1-oxo-1,3a,4,5,6,6a-hexahydro-pentalene-2-carbonitrile (E104);
2-bromo-3-(2-chloro-4-hydroxy-phenyl)-4,5,6,6a-tetrahydro-3aH-pentalen-1-one (E105);
3-(2-fluoro-4-hydroxy-phenyl)-1-oxo-1,3a,4,5,6,6a-hexahydro-pentalene-2-carbonitrile (E106);
2-bromo-3-(5-chloro-2,3-difluoro-4-hydroxy-phenyl)-4,5,6,6a-tetrahydro-3aH-pentalen-1-one (E107);
2-bromo-3-(2,3-dichloro-4-hydroxy-phenyl)-4,5,6,6a-tetrahydro-3aH-pentalen-1-one (E108);
3-(2,3-dichloro-4-hydroxy-phenyl)-4,5,6,6a-tetrahydro-3aH-pentalen-1-one (E109);
3-(5-chloro-2,3-difluoro-4-hydroxy-phenyl)-1-oxo-1,3a,4,5,6,6a-hexahydro-pentalene-2-carbonitrile (E110);
3-(2-chloro-4-hydroxy-phenyl)-1-oxo-1,3a,4,5,6,6a-hexahydro-pentalene-2-carbonitrile (E111);
2-bromo-3-(2-chloro-3-fluoro-4-hydroxy-phenyl)-4,5,6,6a-tetrahydro-3aH-pentalen-1-one (E112);
2-bromo-3-(5-chloro-2-fluoro-4-hydroxy-phenyl)-1-oxo-1,3a,4,5,6,6a-hexahydro-pentalene-2-carbonitrile (E113);
2-bromo-3-(5-bromo-2-chloro-3-fluoro-4-hydroxy-phenyl)-4,5,6,6a-tetrahydro-3aH-pentalen-1-one (E114);
3-(5-chloro-2-fluoro-4-hydroxy-phenyl)-1-oxo-1,3a,4,5,6,6a-hexahydro-pentalene-2-carbonitrile (E115);
3-(2-chloro-3-fluoro-4-hydroxy-phenyl)-4,5,6,6a-tetrahydro-3aH-pentalen-1-one (E116);
3-(2-chloro-3-fluoro-4-hydroxy-phenyl)-1-oxo-1,3a,4,5,6,6a-hexahydro-pentalene-2-carbonitrile E117);
2-bromo-3-(2,6-difluoro-4-hydroxy-phenyl)-4,5,6,6a-tetrahydro-3aH-pentalen-1-one (E118);
3-(2,6-difluoro-4-hydroxy-phenyl)-1-oxo-1,3a,4,5,6,6a-hexahydro-pentalene-2-carbonitrile (E119);
2-bromo-3-(3-chloro-2-fluoro-4-hydroxy-phenyl)-4,5,6,6a-tetrahydro-3aH-pentalen-1-one (E120);
(3aS,6aR)-3-(2,3-difluoro-4-hydroxy-phenyl)-1-oxo-1,3a,4,5,6,6a-hexahydro-pentalene-2-carbonitrile (E121);
(3aR,6aS)-3-(2,3-difluoro-4-hydroxy-phenyl)-1-oxo-1,3a,4,5,6,6a-hexahydro-pentalene-2-carbonitrile (E122);
3-(3-chloro-2-fluoro-4-hydroxy-phenyl)-1-oxo-1,3a,4,5,6,6a-hexahydro-pentalene-2-carbonitrile (E123);
2-bromo-3-(2,3,5-trifluoro-4-hydroxy-phenyl)-4,5,6,6a-tetrahydro-3aH-pentalen-1-one (E124);
2-bromo-3-(3-chloro-2,5-difluoro-4-hydroxy-phenyl)-4,5,6,6a-tetrahydro-3aH-pentalen-1-one (E125);
3-(3-chloro-2,5-difluoro-4-hydroxy-phenyl)-1-oxo-1,3a,4,5,6,6a-hexahydro-pentalene-2-carbonitrile (E126);
2-bromo-3-(2,3,6-trifluoro-4-hydroxy-phenyl)-4,5,6,6a-tetrahydro-3aH-pentalen-1-one (E127);
2-bromo-3-(4-hydroxy-phenyl)-6a-propyl-4,5,6,6a-tetrahydro-3aH-pentalen-1-one (E128);
3-(3,5-difluoro-4-hydroxy-phenyl)-2-ethynyl-4,5,6,6a-tetrahydro-3aH-pentalen-1-one (E129);
3-(2,3-difluoro-4-hydroxy-phenyl)-2-isopropenyl-4,5,6,6a-tetrahydro-3aH-pentalen-1-one (E130);
3-(3,5-difluoro-4-hydroxy-phenyl)-2-isopropenyl-4,5,6,6a-tetrahydro-3aH-pentalen-1-one (E131);
2-Bromo-3-(2,3-difluoro-4-hydroxy-phenyl)-4,5,6,7,8,8a-hexahydro-3aH-azulen-1-one (E132);
or a pharmaceutically acceptable ester, amide, solvate or salt thereof, including a salt of such an ester or amide, and a solvate of such an ester, amide or salt.

6.-7. (canceled)

8. A method for the treatment or prophylaxis of a disease or disorder associated with estrogen receptor activity in a mammal, which comprises administering to the mammal a therapeutically effective amount of a compound of formula (I) as defined in claim 1 or a pharmaceutically acceptable ester, amide, solvate or salt thereof, including a salt of such an ester or amide, and a solvate of such an ester, amide or salt.

9. (canceled)

10. A pharmaceutical composition comprising a compound of formula (I) as defined in claim 1 or a pharmaceutically acceptable ester, amide, solvate or salt thereof, including a salt of such an ester or amide, and including a solvate of such an ester, amide or salt, and a pharmaceutically acceptable carrier.

11. A pharmaceutical composition as claimed in claim 10 further comprising an additional therapeutic agent selected from: an organic bisphosphonate; a cathepsin K inhibitor; an estrogen; an estrogen receptor modulator; an androgen receptor modulator; an inhibitor of osteoclast proton ATPase; an inhibitor of HMG-CoA reductase; an integrin receptor antagonist; anosteoblast anabolic agent; calcitonin; Vitamin D; a synthetic Vitamin D analogue; an anti-depressant; an anxiolytic; or an anti-psychotic; or a pharmaceutically acceptable salt thereof or a mixture thereof.

12. Use of a compound as defined in claim 1 in labelled form as a diagnostic agent for the diagnosis of conditions associated with a disease or disorder associated with estrogen receptor activity.

13. Use of a compound as defined in claim 1 or a labelled form of such a compound as a reference compound in a method of identifying ligands for the estrogen receptor.

14. A method as claimed in claim 8, wherein the condition associated with a disease or disorder associated with estrogen receptor activity is selected from bone loss, bone fractures, osteoporosis, cartilage degeneration, endometriosis, uterine fibroid disease, hot flashes, increased levels of LDL cholesterol, cardiovascular disease, impairment of cognitive functioning, cerebral degenerative disorders, restenosis, gynecomastia, vascular smooth muscle cell proliferation, obesity, incontinence, anxiety, depression, autoimmune disease, inflammation, IBD, IBS, sexual dysfunction, hypertension, retinal degeneration and lung, colon, breast, uterus, and prostate cancer.
